(19) United States
(12) Patent Application Publication
(10) Pub. No.: US 2023/0408493 A1
(43) Pub. Date: Dec. 21, 2023

(54) QUANTIFYING THE RESPONSE-SPECIFICITY OF MONONUCLEAR CELLS AND THERAPEUTIC USES THEREOF

(71) Applicant: The Regents of the University of California, Oakland, CA (US)

(72) Inventors: Alexander HOFFMANN, Los Angeles, CA (US); Katherine May SHEU, Los Angeles, CA (US)

(73) Assignee: The Regents of the University of California, Oakland, CA (US)

(21) Appl. No.: 18/136,072

(22) Filed: Apr. 18, 2023

Related U.S. Application Data
(60) Provisional application No. 63/332,584, filed on Apr. 19, 2022.

(51) Int. Cl. G01N 33/50; C12Q 1/6883; G16H 10/40; G16H 50/20

(52) U.S. Cl. CPC G01N 33/5055; C12Q 1/6883; C12Q 2600/158; G16H 50/20; G16H 10/40

(57) ABSTRACT

The present disclosure describes experimental and analytical methods for the quantification of Response Specificity of immune sentinel cells such as macrophages or monocytes in response to various immune threats or stimuli. The Response Specificity is used as a gauge for the health of the innate immune cells and for guiding therapeutic interventions. The Response Specificity provides a clinical measure for the health of the innate immune system for healthy persons or patients with infectious diseases, autoimmunity, or cancer.

### CROSS-REFERENCE TO RELATED APPLICATIONS

This application claims priority to U.S. Provisional Patent Application Ser. No. 63/332,584, filed Apr. 19, 2022, which is incorporated herein by reference in its entirety.

### STATEMENT REGARDING FEDERALLY SPONSORED RESEARCH OR DEVELOPMENT

This invention was made with government support under Grant Number AI127864, awarded by the National Institutes of Health. The government has certain rights in the invention.

### FIELD

The present disclosure relates in general to the field of immunology. More specifically, the present disclosure describes quantitative analysis of stimulus-specific responses by immune sentinel cells such as macrophages or monocytes and uses thereof.

### BACKGROUND

Immune sentinel cells can sense a wide variety of molecules that derive from viruses, bacteria, fungi, or parasites, termed pathogen-associated molecular patterns (PAMPs), or that are indicative of tissue damage, termed damage-associated molecular patterns (DAMPs). They are recognized by dozens of diverse transmembrane and cytosolic pattern recognition receptors (PRRs). Cytokines produced by first responders, such as TNF or interferons (IFNs), may be sensed through cytokine receptors. Both PRRs and cytokine receptors transmit information about the stimuli via signaling adaptors to a stimulus-responsive signaling network with overlapping downstream pathways consisting of signaling kinases and transcription factors to coordinate diverse immune sentinel functions.

The functions of immune sentinel cells provide for both local anti-microbial activity and systemic immune activation and they coordinate the resolution and tissue healing when the threat is eliminated. Upon exposure to an immune threat, immune sentinel cells may induce resistance factors that may directly limiting pathogen invasion, replication, or assembly. Through secretion of inflammatory cytokines, phospholipids, and second messengers, immune sentinel cells communicate and spread this anti-microbial state to bystander cells within the tissue.

To limit pathogen spread, phagocytic immune sentinel cells, such as macrophages, neutrophils, and dendritic cells, upregulate their ability to engulf pathogens through dramatic reorganization of their cytoskeletons. Production of nitric oxide and reactive oxygen species contributes to pathogen killing, as does the release of antimicrobial DNA-enzyme mixtures, termed NETs (neutrophil extracellular traps). In response to some intra-cellular pathogens, the induction of cell suicide may limit pathogen viability and may occur in the absence of inflammatory mediator release through apoptosis or through inflammation-inducing necroptosis.

Cell-intrinsic pathogen defenses are complemented by the induction of local inflammation and secretion of chemokines for the recruitment and activation of diverse immune effector cells such as neutrophils and NK cells. Secreted and membrane-bound proteases remodel the extracellular matrix and assist migration of immune sentinel cells to the infected site. Eventually, immune sentinel cells orchestrate the adaptive immune response through systemic inflammation and modulatory cytokines that increase antigen presentation, adaptive immune cell production, selection, maturation, and recruitment.

Macrophages are immune sentinel cells that are distributed in every tissue. They recognize a vast variety of immune threats, initiating immune responses that involve innate defenses, sequential recruitment of diverse immune cells to the site of infection, and activation of systemic and adaptive immune mechanisms.

Immune responses are powerful, and often detrimental for the host. Hence, they are deployed on an “only-as-needed” basis. Given that pathogens differ widely in their biology, immune mechanisms that effectively counteract them should differ also. The “only-as-needed” rationale argues that healthy immune sentinel cell responses should be highly specific to the immune threat. Indeed, pathology caused by ineffective immune responses or by immune hyper-activity may be traced to failure of immune sentinel cells to mount immune responses that are specific and appropriate to the particular immune threat. One patho-physiological significance of mis-regulated macrophage responses, is the “cytokine storm” associated with many infections, including COVID-19.

Macrophages are capable of responding to hundreds of distinct pathogens, danger-associated molecular patterns, and cytokines through dozens of distinct receptors, and then generate stimulus-appropriate functions. Indeed, recent studies have revealed that macrophages are capable of producing gene expression programs that are much more stimulus-specific than previously thought.

In view of the important roles played by immune sentinel cells such as macrophages in the innate immune system, there is a need to develop methodologies that would provide quantitative measures for stimulus-specific responses of macrophages in various physiological or pathological conditions.

### SUMMARY

In one embodiment, the present disclosure provides a method of determining innate immune system responsiveness of a subject, comprising (i) obtaining a blood sample from the subject; (ii) exposing monocytes or monocyte-derived cells (e.g., macrophages, dendritic cells) derived from the blood sample to one or a combination of stimuli such as cytokines, pathogen-associated molecular patterns, and damage-associated molecular patterns; (iii) determining using genome wide RNA profiling, single cell mRNA expression of one or more of stimuli-related genes, or protein expression, responses to the stimuli by the monocytes or monocyte-derived cells (e.g., macrophages, dendritic cells); and (iv) determining a response specificity score characterizing the innate immune system responsiveness of the subject, wherein the determination comprises use of information theoretic and machine learning methodologies.

In another embodiment, the present disclosure provides a method for treating a subject having a condition or disease, comprising the steps of: (a) determining the responsiveness of the subject's innate immune system by a method comprising: (i) obtaining a blood sample from the subject; (ii) exposing monocytes or monocyte-derived cells (e.g., macrophages, dendritic cells) derived from the blood samples to one or a combination of stimuli such as cytokines, pathogen-associated molecular patterns, and damage-associated molecular patterns; (iii) determining using genome wide RNA profiling, single cell mRNA expression of one or more of stimuli-related genes, or protein expression, responses to the stimuli in the monocytes or monocyte-derived cells (e.g., macrophages, dendritic cells); and (iv) determining a response specificity score characterizing the responsiveness of the subject's innate immune system, wherein the determination comprises use of information theoretic and machine learning methodologies; (b) using the subject's response specificity score to query an information repository comprising (i) innate immune system responsiveness of a plurality of healthy subjects and patients having the condition or disease, and (ii) a correlation between the innate immune system responsiveness and a therapeutic outcome of the patients to one or more therapeutic regimens, and identifying a correlation value for the subject; and (c) treating the subject with a therapeutic regimen when the correlation value indicates a positive therapeutic benefit for the subject; or avoiding treating the subject with a therapeutic regimen when the correlation value indicates an absence of or a negative therapeutic benefit for the subject.

These and other aspects of the invention will be appreciated from the ensuing descriptions of the figures and detailed description of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 shows that macrophages are immune sentinel cells that respond stimulus-specifically. Innate immune macrophages have pattern-recognition receptors and cytokine receptors capable of sensing pathogens stimulus-specifically. As sentinel cells, they response to thousands of possible pathogen or damage-related immune threats. Many host defense functions may also be detrimental to the host, and it is thus advantageous that they be deployed carefully.

FIG. 2 (with FIGS. 3 and 4A-B) depicts an approach for quantifying stimulus-response specificity. FIG. 7 shows macrophages are exposed to a variety of immune ligands representing Gram-negative or -positive bacteria, viral nucleic acid, or host cytokines. Single cell RNAseq data is collected over time points using the Rhapsody system, and a set of 500 immune response genes are sequenced.

FIG. 3 depicts motif enrichment of all induced genes vs gene selected for the Rhapsody panel shows further enrichment of AP1, NFκB and IRF pathway target genes.

FIGS. 4A and 4B show relative patterns of expression recapitulated between published bulk data and single cell data.

FIGS. 5A-5B show that Expression variances are gene-, stimulus-, and timepoint-specific. FIG. 5A shows three examples of cytokine or feedback regulator genes at multiple timepoints. Green points represent the mean. FIG. 5B shows quantification of mean and Fano factor for each stimuli for the three genes. Increases in Fano factor indicate greater variability for that timepoint.

FIGS. 6A-6D show that gene expression heterogeneity hampers response specificity. FIG. 3. FIG. 6A shows channel capacity (CC) schematic. FIG. 6B shows CC of individual genes with heatmap of expression. FIG. 6C shows Examples from FIG. 6B.

FIG. 6D shows CC of pairwise stimulus comparisons: bacterial vs cytokine, bacterial vs bacterial, bacterial vs viral. For specificity of CpG vs pIC, a zoom-in of the mean and variance contribution to response specificity. Response specificity of Cxcl10 is low due to high heterogeneity, even though mean expression values are distinct.

FIGS. 7A-7B show that different biological functions show distinct response specificities. FIG. 7A shows cell intrinsic antiviral (Ifit3, Mx1, Mx2, Oas11) and systemic immune activating pro-inflammatory (Tnf, I16, I11a, I11b) categories both have the highest stimulus-specificity. FIG. 7B shows CC of Cell death (Bc12a1a, Bc12a1d, Bc12111, Tnfaip3) and Antioxidant genes (Gsr, Sod2, Gc1m, Gsta3) for pairwise stimuli comparisons. Low expression variance of cell death genes during LPS stimulation results in high CC for LPS vs host or viral ligands.

FIGS. 8A-8F show that M1 and M2 polarization affect response specificity differently. FIG. 8A shows data collection schematic. FIG. 8B shows PCA of M0, M1, M2 macrophage responses at 8 hrs. FIG. 8C shows motif enrichment of WGCNA modules; ME1 is an IRF module. FIG. 8D shows individual genes in the interferon pathway (ME1 by WGCNA), lose specificity in M1 macrophages. FIG. 8E shows CC for example gene Cxcl10 in M0, M1, M2 over time. FIG. 8F shows violins for Cxcl10 in M0, M1, M2.

FIGS. 9A-9E depict the genes most informative of response specificity are not cytokines. FIG. 9A shows CC of the best possible combinations of n genes, up to 20 dimensions. At 3 and 8 hrs, 5 genes already exceeds 2 bits, corresponding to a capacity to distinguish 4 stimuli. FIG. 9B shows best combination of genes for 2, 5, 10 dimensions. FIGS. 9C-D show random forest classifier to predict stimuli, using either all genes or a subset of genes identified by channel capacity analysis: FIG. 9C, the top 15 genes; FIG. 9D; the top 2 genes. FIG. 9E shows FDR using a subset of genes (middle panel) is comparable to all genes (left panel) and better than random sets of 15 genes (right panel).

FIGS. 10A-10F describe the development of a Response Specificity Index to assess the health of innate immune cells and developing a scoring method for response specificity. FIG. 10A shows Bhattacharyya distance between each stimulus and all others for M0, M1, M2 to identify informative stimulus subsets. For each pair of stimuli, left to right, M0, M1, M2. FIG. 10B shows channel capacity calculated on the first 3 PCs of M0, M1, M2 macrophages forms the Index. For each pair of stimuli, left to right, M0, M1, M2. FIG. 10C shows applying the Index to young (16 weeks), old (>90 weeks), or obese (16 weeks) mice peritoneal macrophages (PM). FIG. 10D shows tSNE of PM responses. FIG. 10E shows distance metric for PMs after projection onto M0, M1, M2 PCA. For each pair of stimuli, left to right, LFD, Old, HFD. FIG. 10F shows more M2-like IFNβ and TNF response in old PMs. Decreased response specificity in PMs from high-fat diet obese mice due to TNF & pIC mixing.

FIGS. 11A-11F show macrophages produce highly specific gene expression responses to diverse immune stimuli. FIG. 11A shows a cost-effective experimental workflow using targeted single-cell RNA-sequencing to profile immune response gene expression at single cell resolution. FIG. 11B depicts comparison of bulk RNAseq data and single cell data, showing concordance of gene expression clusters, and also reveals single cell heterogeneity. FIG. 11C depicts PCA that indicates there may be a high degree of stimulus-specificity despite single cell heterogeneity, but TNF, P3C and CpG appear overlapping on the first two components. Ellipses represent 95% confidence intervals based on multivariate t-distribution. FIG. 11D shows UMAP on the top 20 PCA components clarifies that TNF can be separated, but P3C and CpG response distributions remain overlapping. FIG. 11E shows calculation of the Bhattacharya distance between pairs of response distributions confirms the similarity of CpG and P3C distributions. IFNβ is the most distinct from all other stimuli, but with a response-distribution space closest to that of PIC. Bhattacharya distances were calculated on the top 20 principal components. FIG. 11F depicts a random forest classifier confirms high identifiability of each stimulus condition, with only the bacterial ligands P3C and CpG being confused with each other. Classifier was trained on 70% of all single cell data (2 replicate experiments from M0 naïve macrophage populations) and tested on the remaining 30% of held-out data.

FIGS. 12A-12D show selection of targeted gene panel and stimuli. FIG. 12A: Top panel: Heatmap of all genes induced with log₂ (FC)>2 in any stimulus from bulk RNAseq data (Cheng et al, 2017). Middle panel: PCA loadings from bulk RNAseq data. Genes were selected by scoring each gene based on loadings from the top 20 PCs. Bottom panel: Selected genes are marked on the full heatmap. FIG. 12B: Enriched motifs found in all 1502 induced genes vs. 500 genes selected for target amplification. FIG. 12C: Tensor components analysis on all stimuli, all timepoints. Red boxes highlight stimuli chosen. FIG. 12D: PCA performed on all data points together. Pairwise Euclidean distances between points in PCA space are calculated for each timepoint.

FIGS. 13A-13D show comparison of single-cell RNAseq platforms, Rhapsody vs FIG. 13A: Heatmap of bulk RNAseq, and pseudobulk values from Rhapsody or 10×, for the same set of genes. FIG. 13B: Violin plots of scRNAseq data for a few example genes for Rhapsody vs 10×. FIG. 13C: Distributions of genes measured per cell (top row), or counts per gene (bottom row), comparing Rhapsody (left), 10× for all genes (middle), and 10× for the 500 genes in the Rhapsody panel (right). For genes detected by both platforms, there are similar distributions in the number of genes per cell and the number of read counts per gene, and most genes outside the custom panel had low or zero read counts. FIG. 13D: Left panel: Violin plot of percentage of cells with 0 counts for each gene in the 10× or Rhapsody platforms. 100% indicates that all cells are measured at 0 for that gene. Right panel: Scatterplot comparing 10× vs Rhapsody “percentage of 0's per gene”, across all common genes.

FIGS. 14A-14B show reproducibility of the macrophage experimental system and measurement platform. FIG. 14A: Scatterplot of pseudobulk values across 5 stimuli and 2 timepoints for 2 replicates from the Rhapsody scRNAseq platform. Rhapsody replicates of the single-cell data for five stimuli were concordant in means (avg. Pearson's r=0.86). FIG. 14B: Single-cell distributions for example genes from each replicate, indicating concordant stimulus-specific patterns in response distributions.

FIGS. 15A-15C show machine learning test probabilities and top gene combinations. FIG. 15A: Distribution of prediction probabilities across cells for the test data. Based on its transcriptome, each cell in the test set was given a probability of having encountered a particular ligand, and the highest probability ligand was assigned as the prediction. Prediction probabilities helped distinguish weak versus strong assignments: for instance, correct IFNβ predictions consistently had prediction probabilities close to 1, whereas correct CpG predictions had prediction probabilities of ˜0.6-0.7 with the second-best choice being P3C. FIG. 15B: Comparison of overall accuracy for three different model types show similar overall accuracies, resulting in a similar prediction accuracy from ensemble-based majority voting from all three models. Left to right: nnet, rf, kknn, ensemble. FIG. 15C: Gene expression values for the top gene combinations providing the greatest ligand discrimination, as quantified by max MI, for M0 macrophages using 1, 2, or 3 genes. Units are log 2 (normalized counts+1).

FIGS. 16A-16G show that despite high stimulus-specificity of gene expression programs, individual genes are capable of distinguishing only a subset of stimulus pairs. FIG. 16A: An information theoretic approach treats single cell signaling and epigenetic mechanisms as a communication channel that passes extracellular information to nuclear target genes. FIG. 16B: Distribution of max MI values for single genes shows that only a few genes have a max MI above 1 bit, with 1 bit indicating the ability to distinguish two groups. FIG. 16C: Heatmap of single cell gene expression indicates that low max MI is because most genes have binary expression patterns, on or off, rather than a range of levels that would have been able to distinguish multiple groups of stimuli. FIG. 16D: Max MI as a function of the indicated number of genes in combination, for the gene combinations providing the highest max MI. FIG. 16E: Gene names for the combinations that allow for the highest max MI for each given number of genes. FIG. 16F: Random forest classifier for gene combinations shows that a small number of genes generates fair prediction accuracy, and a classifier trained on 15 genes performs approximately the same as all genes. FIG. 16G: The genes comprising the top 2-gene combination, Cmpk2 and Nfkbiz, distinguish complementary stimulus-pairs. Error bars represent standard deviations from 10 iterations of 50% bootstrap resampling of the single cell data.

FIGS. 17A-17E show the Response Specificity of cytokine genes is limited by cell-to-cell heterogeneity, despite having distinct expression means. FIG. 17A: Deviation in mean expression among the six stimuli plotted against max MI for each gene. Genes are colored by their average amount of dispersion across the six stimuli (average Fano Factor). FIG. 17B: Average Fano Factor across the six stimuli plotted against max MI shows genes with high or low dispersion. Genes are colored by deviation in mean expression across stimuli (mean squared deviation). FIG. 17C: Mean squared deviation vs. average Fano factor over all stimuli, with cytokine genes highlighted. FIG. 17D: Expression distributions of cytokine (Cc15, Tnf, Cxcl10) and non-cytokine (Cmpk2) genes, showing distinct variance and modality of responses to each stimulus. FIG. 17E: Mean expression level vs. expression variance for each stimulus-response distribution. Dotted lines mark where variance equals mean.

FIGS. 18A-18F show stimulus specificity of immune response genes from selective deployment of IRF and p38 pathways, and NFκB dynamical features. FIG. 18A: Signaling and gene regulatory mechanisms are responsible for generating Response Specificity. MAPKp38, IRF3, AP1, and NFκB signaling profiles are activated in response to inflammatory stimuli and act in combination to regulate gene expression, using five identified regulatory logics (Cheng et al., 2017). FIG. 18B: Distribution of max MI values for genes of the five regulatory logics, for IFNβ vs. TNF. FIG. 18C: Distribution of max MI values for genes of P3CSK vs. TNF. FIG. 18D: Distribution of max MI values for genes of P3CSK vs. PIC. FIG. 18E: Single-cell NFκB signaling dynamics in response to TNF, P3CSK, LPS, or PIC. Information conveyed by NFκB dynamical activity is transmitted through six identified NFκB signaling codons (Adelaja et al., 2021). FIG. 18F: Response Specificity of NFκB target genes across all pairs of stimuli are associated with stimulus-specificity in select NFκB signaling codons.

FIGS. 19A-19G show relationship between IRF and MAPKp38 pathway deployment, or NFκB signaling dynamics, and gene expression specificity. FIG. 19A: Max MI for response distributions of single genes, for LPS vs. P3CSK. FIG. 19B: Max MI for response distributions of single genes, for TNF vs. PIC. FIG. 19C: Max MI for response distributions of single genes, for LPS vs. PIC. FIG. 19D: Max MI for response distributions of single genes, for LPS vs. TNF. FIG. 19E: Pairwise max MI values for Cxcl10 expression is most correlated with the NFκB signaling codon Total Activity, rather than the codons Oscillations (FIG. 19F) or Duration (FIG. 19G). x- and y-axes are the pairwise max MI values for the indicated signaling codon, and the indicated gene.

FIGS. 20A-20E show polarized macrophages express the appropriate polarization markers. FIG. 20A: PCA of M0, M1, and M2 macrophages at baseline 0 hrs. M1 and M2 macrophages have been treated with IFNγ or IL4 for 24 hrs. FIG. 20B: Macrophage marker gene Adgre1 is expressed in all three conditions. FIG. 20C: M1 marker Cd86 at 0 hr. M1 marker Nos2 or Cxcl10 at 0.25-0.5 hour, combining cells from all stimuli. FIG. 20D: M2 marker genes at 0 hrs. FIG. 20E: Left panel: PCA loadings from PCA on M0, M1, M2 cells together. Right panel: UMAP of M0, M1, M2 cells together, using the top 20 principal components.

FIGS. 21A-21F show cytokine polarization causes complex modulation of the Response Specificity of specific genes to specific stimuli. FIG. 21A: The Response Specificity experimental assay captures response distributions to six stimuli in M1 (IFNγ) and M2 (IL4) polarized macrophages. FIG. 21B: PCA on all M0, M1 (IFNγ), and M2 (IL4) macrophage responses to the six stimuli. Variance explained by PC1 (19.9%), by PC2 (15.7%). Polarized macrophages produce response distributions distinct from those of M0 macrophages. FIG. 21C: Response Specificity as calculated by max MI is slightly reduced in both M1 (IFNγ) and M2 (IL4) polarization states, for every dimension of best possible gene combinations. FIG. 21D: Genes within the best 15-gene combination for M0, M1 (IFNγ), and M2 (IL4) macrophages are distinct. FIG. 21E: Top panel: Distribution of max MI values of individual genes for M0, M1 (IFNγ), and M2 (IL4) macrophage responses. Dotted lines mark the max MI of Cxcl10 for each macrophage state: red (M0), green (M1), blue (M2). Bottom panel: Cxcl10 stimulus-response distributions for M0 (left), M1 (center) and M2 (right) macrophage states are shown. FIG. 21F: Scatterplot of differences in max MI values between M0 vs. M1 (IFNγ) or M2 (IL4) macrophage responses. Genes are colored by assigned gene regulatory logic.

FIG. 22 shows stimulus-specific genes and changes to their Response Specificity in polarized macrophages. Left panel: Correlation of the change in max MI for M1 vs M2 macrophages, each compared to M0, for all genes. Right panel: Same as the left panel with points colored by gene regulatory strategy.

FIGS. 23A-23C show the Response Specificity Profile of stimulus pairs assesses the functional state of macrophages, and readily distinguishes M0 vs. M1 vs. M2. FIG. 23A: Response Specificity Profiles measured by max mutual information of all 15 stimulus pairs, as defined by response distributions to 6 stimuli. M0, M1, and M2 macrophage responses each show specific differences in select pairs. Error bars represent standard deviations from 50 iterations of 50% bootstrap resampling. FIG. 23B: Across all pairs, the difference in max MI from M0 responses is calculated to derive delta Response Specificity Index, a characteristic signature of the functional state of the macrophage. FIG. 23C: A succinct summary of the pair-wise profile into a single number, the delta Response Specificity Index, provides a clearer indication of differences than calculating the mutual information on all stimulus-response datasets together. Error bars for ΔRSI represent propagation of the original standard deviations in max MI calculations.

FIGS. 24A-24E show old and obese mouse peritoneal macrophages show distinctive alterations in their Response Specificity Profile. FIG. 24A: Measuring Response Specificity for three mouse models using peritoneal macrophages: Healthy low-fat diet mice (16 wks), old mice (>90 wks), and high-fat diet mice (16 wks). Cells from two mice were aggregated for each condition. FIG. 24B: tSNE visualization of macrophage responses to stimuli for each of the mouse models, healthy (LFD), old, and high-fat diet (HFD), using the top 20 principal components. FIG. 24C: Macrophage responses from healthy and disease mouse models are scored by the Response Specificity Profile. Error bars represent standard deviations from 50 iterations of 50% bootstrap resampling. Subtracting each profile from the Response Specificity Profile of M0 macrophages highlights pair-wise differences in max MI values. FIG. 24D: Square root of the sum of the squared deviation from M0 across all stimulus-pairs was calculated to obtain the single-value ΔRSI (FIG. 6b). Subsets that included CpG were not included due to CpG not being used in the Response Specificity assay on peritoneal macrophages. Higher numbers for ΔRSI indicate a larger deviation in Response Specificity Index from M0. Error bars represent propagation of the original standard deviations in max MI calculations. FIG. 24E: Scatterplot of differences in max MI values between LFD vs. Old or HFD macrophage responses. Genes are colored by absolute max MI quantity for each gene in the LFD mouse model.

FIGS. 25A-25D show time-series single cell RNAseq allows evaluation of time-dependent macrophage responses. FIG. 25A: Time-series scRNAseq data collection scheme for three macrophage states responding to six different immune ligands. FIG. 25B: Macrophage response potentials are revealed by ligand stimulation, and responses are canalized towards different functions by microenvironmental cytokines. FIG. 25C: Heatmaps of all scRNAseq measurements of M0, M1 (IFNγ), and M2 (IL4) macrophage responses. Each column is the gene expression profile of a single cell. Timepoint, stimulus, and polarization condition of the cell is marked. FIG. 25D: Violin plots of single-cell Tnf expression, showing stimulus-response distributions over time for each macrophage state and each stimulus. Green points represent median.

FIGS. 26A-26C show cell counts and data quality. FIG. 26A: Number of single cells collected for each sample. Each sample is a combination of timepoint, stimulus, and macrophage polarization condition. Range of cell numbers per sample: 390-7215 cells. FIG. 26B: Heatmaps of pseudobulk values calculated from scRNAseq are concordant across replicates at the matching timepoints. Each column is pseudobulk RNAseq data from a timepoint and stimulus. Genes are clustered in the same order as FIG. 25C. FIG. 26C: Combining all timepoints from both scRNAseq replicate experiments results in pseudobulk gene expression clusters that match clusters from population-level bulk RNAseq.

FIGS. 27A-27B show heterogeneity of induced gene expression in individual genes. Stimulus-response gene expression distributions for Ccl5 and Cxcl10 are shown for each macrophage polarization state and each stimulus across timepoints. Green points represent median. Y-axis represents log 2(counts+1).

FIGS. 28A-28E show modeling an ensemble of single-cell expression trajectories from time-series scRNAseq data. FIG. 28A: Schematic of imputation of single cell expression trajectories from time-series scRNAseq. FIG. 28B: Schematic of the scREAL-TIME algorithm: 1) Stimulate/perturb the cell population and collect time-series scRNAseq measurements. 2) Perform PCA on time-series scRNAseq data to embed gene expression information into cell scores, and cluster single-cells into cell archetypes. 3) Performed weighted random walks through cell archetypes in PC space, weighted by distance of clusters across two timepoints, as well as cluster density. Random walks that are weighted makes certain trajectories more common (bolded connecting lines), and outlier trajectories possible but infrequent (dashed connecting lines). 4) Perform spline interpolation over unmeasured time points to obtain cell trajectory splines at each PC. 5) Multiply cell trajectory space by PCA loadings matrix to recover gene expression trajectories for each cell. FIG. 28C: Left panel: Measured time-series data for Tnf does not allow tracking of single cells over time. Right panel: scREAL-TIME recovers true-time ensembles of Tnf expression trajectories for M0 macrophages across all stimuli. Trajectories are displayed as a heatmap scaled to max expression for each gene across all stimuli. FIG. 28D: Hierarchical clustering of single cells using information from the entire trajectory of Tnf expression. FIG. 28E: Hundreds of genes are measured per cell with distinct gene-gene correlation patterns. Hierarchical clustering of the trajectories of multiple genes per cell further distinguishes the stimuli.

FIGS. 29A-29D show comparison of mean and variances of modeled trajectory ensembles vs. data for each gene. FIG. 29A: Gene expression means from imputed trajectories are concordant with means at measured timepoints. Normalized RMSD (nRMSD) for each gene is calculated using z-scored values across all timepoints and all stimuli and indicates that some trajectory imputations match the data more closely. FIG. 29B: Gene expression variances from imputed trajectories are concordant with variances at measured timepoints. FIG. 29C: Distribution of mean nRMSDs across all genes, with Tnf and Cxcl10 marked by horizontal dotted lines. FIG. 29D: Distribution of variance RMSDs across all genes, with Tnf and Cxcl10 marked by horizontal dotted lines.

FIGS. 30A-30C show quality control of modeled trajectory ensembles. FIG. 30A: Left panel: Trajectory ensembles for Tnf imputed from scRNAseq data via scREAL-TIME. Center panel: Violin plots of imputed gene expression distributions for Tnf at matching timepoints as measured data. Right panel: Violin plots of gene expression distributions for Tnf from measured scRNAseq data. FIG. 30B: Same as FIG. 30A for Ccl5. FIG. 30C: Same as FIG. 30A for Cxc10.

FIGS. 31A-31C show clustering on timepoint data does not distinguish stimuli. FIG. 31A: Hierarchical clustering of single cells using only information from single timepoints of Tnf expression. Each row is a single cell stimulated with a ligand. Clustering was performed by setting all unmeasured values to zero. Heatmap color bar represents log 2(counts+1). FIG. 31B: Heatmap of expression values of five genes from scRNAseq data taken at different timepoints. Heatmap color bar represents log 2(counts+1). FIG. 31C: Hierarchical clustering of single cells using only information from single timepoints of expression for 5 genes in combination. Clustering was performed by setting all unmeasured values to zero. Heatmap color bar represents log 2(counts+1).

FIGS. 32A-32G show unsupervised dimensionality reduction reveals specificity in single-cell expression trajectories. FIG. 32A: Population level transcriptomic data shows stimulus-specific gene expression patterns in macrophage responses over an 8 hour time-course. FIG. 32B: Tensor components analysis of bulk RNAseq data using Tucker decomposition. FIG. 32C: Average stimulus-responses are unique when considering their entire time-course, but some stimuli produce very similar average response patterns. Dotted lines represent the distributions of potential single cell macrophage responses, not evident in population level data. FIG. 32D: Single-cell expression trajectories obtained through scREAL-TIME allow tensor decomposition on thousands of single cells over a time-course. FIG. 32E: The amount of stimulus-information contained in the top few components of the tensor decomposition. Max MI=2.4 bits on 4 components. FIG. 32F: TCA on single cells across all stimuli, where using single cell trajectory ensembles reveals the distinguishability of single cell distributions. FIG. 32G: Plotting the temporal factors in the top five components of the decomposition indicates that distinct dynamical patterns of activity may be responsible for the observed stimulus-distinguishability.

FIGS. 33A-33D show cell-to-cell heterogeneity in the dynamical features of inducible gene expression. FIG. 33A: Gene expression trajectory dynamical features that may be important for innate immune responses. FIG. 33B: Tnf expression trajectories allow analysis of dynamical features distributions in single cells across all stimuli. FIG. 33C: Analysis of stimulus-information in time-points vs dynamical features for all individual genes. Features such as “integral” on average hold more stimulus-information than other dynamical feature values or expression values at any single timepoint. FIG. 33D: The gain in information due to having the full expression trajectory differs for each gene regulatory strategy.

FIGS. 34A-34E show single-cell gene-gene correlations of dynamical features across polarization states. FIG. 34A: Correlation of Tnf vs. Nfkbia across timepoints. FIG. 34B: Correlation of Tnf vs. Nfkbia Integral (total activity). FIG. 34C: Correlation of Tnf vs. Cxcl10 across timepoints. FIG. 34D: Correlation of Tnf vs. Cxcl10 Integral (total activity). FIG. 34E: Hierarchical clustering of correlation heatmaps across all genes, using expression values at the 3 hr timepoint or Integral values from single cell trajectories. Genes do not cluster as well by regulatory strategy when analyzing gene-gene correlations at a single timepoint.

FIGS. 35A-35C show quality control of modeled trajectory ensembles for M1 (IFNγ) macrophages. FIG. 35A: Left panel: Trajectory ensembles for Tnf imputed from scRNAseq data via scREAL-TIME. Center panel: Violin plots of imputed gene expression distributions for Tnf at matching timepoints as measured data. Right panel: Violin plots of gene expression distributions for Tnf from measured scRNAseq data. FIG. 35B: Same as FIG. 35A for Ccl5. FIG. 35C: Same as FIG. 35A for Cxc10.

FIGS. 36A-36C show quality control of modeled trajectory ensembles for M2 (IL4) macrophages. FIG. 36A: Left panel: Trajectory ensembles for Tnf imputed from scRNAseq data via scREAL-TIME. Center panel: Violin plots of imputed gene expression distributions for Tnf at matching timepoints as measured data. Right panel: Violin plots of gene expression distributions for Tnf from measured scRNAseq data. FIG. 36B: Same as FIG. 36A for Ccl5. FIG. 36C: Same as FIG. 36A for Cxc10.

FIGS. 37A-37E show comparison of dynamical feature heterogeneity and specificity across polarization states. FIG. 37A: Hierarchical clustering of trajectories for all M1 (IFNγ) and M2 (IL4) cells compared to M0 (center), using a subset of cytokine and feedback regulator genes. FIG. 37B: Max mutual information about the stimulus for polarized macrophages at individual timepoints. FIG. 37C: Max mutual information about the stimulus for polarized macrophages considering dynamical features. FIG. 37D: Identification of most informative gene combinations for “Integral” (solid lines) across polarization states, in comparison to the best gene combinations for the 3 hr timepoint (dotted lines). Inset displays genes selected as most informative in combination. FIG. 37E: Scatterplot of best 3-gene combination for “Integral”, or total activity, of single-cell expression trajectories for each polarization state.

FIGS. 38A-38E show comparison of polarization states. FIG. 38A: Trajectory dynamical feature distributions for different macrophage polarization states for Tnf. FIG. 38B: Trajectory dynamical feature distributions for different macrophage polarization states for Cxcl10. FIG. 38C: Quantification of variance/mean (Fano Factor) for “Integral” across polarization states and stimuli. Left panel: Tnf. Right panel: Cxcl10. FIG. 38D: Quantification of variance/mean (Fano Factor) for “max LFC” across polarization states and stimuli. Left panel: Tnf. Right panel: Cxcl10. FIG. 38E: Hierarchical clustering of single cell expression trajectories of best 3-gene combination for M0 Integral, compared to the same genes for M1 (IFNγ) and M2 (IL4) macrophages.

FIGS. 39A-39F show Response Specificity Profiles identify dynamical features that lose stimulus-specificity in each polarization state. FIG. 39A: Dynamics-based Response Specificity Profile, which is the pairwise max MI of all possible pairs of stimuli. Difference in max MI between M0 and M1 (IFNγ) or M2 (IL4) polarization states, for each pair of stimuli, are calculated over the first three principal components of each dynamical feature. FIG. 39B: Delta response specificity index (ΔRSI) for each feature, which is the root mean square of all pairwise deviations of polarized macrophages from M0 macrophages. FIG. 39C: Highest PCA loadings identifies genes contributing to loss of specificity in stimulus-pairs via Max LFC. x-and y-axes are drawn to scale. FIG. 39D: Yid Max LFC distributions for M0 vs. M1 (IFNγ) macrophages. FIG. 39E: Highest PCA loadings identifies genes contributing to loss of specificity in stimulus-pairs via Speed. x-and y-axes are drawn to scale. FIG. 39F: Mx1 Speed distributions for M0 vs. M2 (IL4) macrophages.

FIGS. 40A-40C show identification of genes whose response dynamics indicate cell state. FIG. 40A: Cell states can be predicted from steady-state unstimulated transcriptome, or by transcriptome dynamics Top 3 genes identified by max MI from steady-state scRNAseq profiles, and the integral of the same 3 genes after CpG stimulation for 8 hrs. While the M1 state is well distinguished at both steady-state and with response dynamics, M0 and M2 states are much more clearly distinguished by response dynamics. FIG. 40B: F1 scores for classifying macrophage states, using the top 5 genes identified by max MI, for each stimulus-response. Classification accuracy for cell state is higher when considering dynamical features over single-timepoints, and higher when considering a response timepoint over steady-state (0 hours). FIG. 40C: Multinomial regression using LASSO-penalization shows that fewer genes are needed to produce accurate predictions when dynamical features are considered. Single timepoints responses are still more informative than steady-states (0 hrs).

FIGS. 41A-41C show dynamics of marker genes are better than steady-states in distinguishing polarization states. FIG. 41A: Time-series scRNAseq of M1 macrophage marker Nos2. FIG. 41B: Scatterplots of three canonical M1 or M2 marker genes at steady-state (unstimulated), vs. the integral of their expression dynamics FIG. 41C: F1 score of classification accuracy when considering the three marker genes for unstimulated macrophages, stimulus-response timepoints, or stimulus-response dynamics/dynamical features.

### DETAILED DESCRIPTION

The present disclosure describes methods to measure Response Specificity (RS) (also referred to herein as Response Specificity Index (RSI) or Stimulus-Response Specificity) of monocytes or monocyte-derived cells (such as macrophages and dendritic cells) by assessing the response of such cells ex vivo or in vitro to various stimuli. By quantifying the Response Specificity, one can ascribe a Response Specificity score to monocytes-derived cells from different physiological or pathological conditions and identify its molecular drivers to address both fundamental biological questions and advance clinical studies, including the establishment of normal Response Specificity score ranges and detection of changes due to disease, and the effectiveness of or contraindications to treatments thereof. Response Specificity is indicative of a subject's innate immune health in guarding against many diseases and insults, and the assessment of Response Specificity score is disclosed herein as a useful clinical measurement, for example, to assess a patient with or at risk of inflammatory disease, or candidates for therapeutic interventions, to guide therapy for cancer or cancer immunotherapy, transplant rejection, and infectious diseases, by way of non-limiting examples.

The conceptual innovation is that hallmark of innate immune cells is Response Specificity. Response Specificity is provided herein as a clinical measure of innate immune health that will benefit people with pre-existing conditions that put them at risk for, e.g., infections or autoimmunity and in particular whether a particular treatment will be effective, be ineffective or even cause adverse consequences. Response Specificity can be measured by the experimental and analytical methods described herein.

In one embodiment, the Response Specificity is quantified by first tracking the activity and/or expression of one or more immune response genes at single cell resolution in a plurality of, e.g., hundreds of macrophages or monocytes, at a single time point or over time, after exposure to a panel of stimuli, individually or in combination. The immune response genes are selected to capture diverse responses to various immune stimuli described herein. In one embodiment, the present specification uses either the 10X Genomics systems for genome-wide profiling or the BD Rhapsody system for single cell profiling the expression of 500 immune response genes. In one embodiment, protein expression or upregulation is measured. In one embodiment, a combination of RNA and protein expression is assessed, which may be from different genes or in some embodiments from the gene. From analysis of multiple samples, an optimal time after stimulation for assessing effect on immune response genes may be selected to provide a standard method for obtaining the expression data. Such assessment may then be applied to each of the stimuli and each of the assessed expressions, to identify optimal time point after stimulation to measure the response. As a result, a single time point after exposure of macrophages to the panel of stimuli is selected such that a facile assessment routine is established for measuring Response Specificity.

Thus, in one embodiment, the present disclosure provides a method of determining innate immune system responsiveness of a subject, comprising (i) obtaining a blood sample from the subject; (ii) exposing monocytes or monocyte-derived cells (e.g., macrophages, dendritic cells) derived from the blood samples to one or a combination of stimuli such as cytokines, pathogen-associated molecular patterns, and damage-associated molecular patterns; (iii) determining responses using genome wide RNA profiling, single cell mRNA expression of one or more of stimuli-related genes, or protein expression, in response to the stimuli in the monocytes or monocyte-derived cells (e.g., macrophages, dendritic cells); and (iv) determining a response specificity score characterizing the innate immune system responsiveness of the subject, wherein the determination comprises use of information theoretic and machine learning methodologies.

In one embodiment, the blood samples comprise peripheral blood mononuclear cells. In one embodiment mononuclear cells from the blood sample are analyzed. In one embodiment mononuclear cells are differentiated into macrophages or dendritic cells. In one embodiment macrophages are analyzed. In one embodiment dendritic cells are analyzed.

In one embodiment, the exposing is to single cells.

In one embodiment, the determining responses is at a single time point after exposing, or at a plurality of time points after exposing. In one embodiment, the plurality of time points includes a zero time point. In one embodiment, the plurality of timepoints are within about 24 hours of exposing. In one embodiment, the plurality of timepoints are within about 12 hours of exposing. In one embodiment, the plurality of timepoints are within about 8 hours of exposing. In one embodiment, the plurality of timepoints are within about 3 hours of exposing. In one embodiment, the exposing after multiple timepoints comprises 0, 0.5, 1, 3, 5 and 8 hours after exposing. In one embodiment, the exposing after multiple timepoints consists of 0, 0.5, 1, 3, 5 and 8 hours after exposing. In one embodiment, the plurality of timepoints are within about 3 hours of exposing. In one embodiment, the determining the response specificity score is based on a plurality of time points after exposing. In one embodiment, the plurality of timepoints of exposing includes an exposing with no stimuli.

In one embodiment, the determining responses for a plurality of timepoints comprises integral, fold change, peak amplitude, speed, or any combination thereof.

Examples of cytokine stimuli include, but are not limited to, TNF, IFNγ, IL-6, IL-2 or any combination thereof. Examples of pathogen-associated molecular patterns stimuli include, but are not limited to, Pam3CSK4 (abbreviated P3CSK4), CpG, LPS, polyI:C, flagellin or any combination thereof. In one embodiment, the pathogen-associated molecular patterns stimuli are from fungi, protozoa, helminths, bacteria, viruses, or any combination thereof. Examples of damage-associated molecular patterns stimuli include, but are not limited to, products derived from lysed cells, necrotic cells, injured cells, or cells treated with heat (e.g., heat shock) or pressure. In one embodiment, the combinations of aforementioned stimuli may be applied to cells.

In one embodiment, the stimuli comprise LPS, polyI:C, IFNβ, P3CSK4, CpG and TNF. In one embodiment, the stimuli consist of LPS, poly I:C, INFβ, P3CSK4, CpG and TNF.

In one embodiment, a no stimulus control is included. In one embodiment, the stimuli-related genes can be IkBa, p-RelA, pSTAT1, pSTAT2, pSTAT3, pSTAT5, p-IRF3, p-p38, p-ERK, p-JNK, or any combination thereof. Single-cell RNA expression is determined. In one embodiment, antibodies to stimulus-responsive proteins such as IkBa, p-RelA, pSTAT1, pSTAT2, pSTAT3, pSTAT5, p-IRF3, p-p38, p-ERK, p-JNK, may be used, singly or any combination thereof.

In one embodiment, the stimuli-related genes comprise Tnf, 116, Cc15, Cc110, Nfkbia, or any combination thereof.

In one embodiment, the top 5 macrophage polarization state-related genes which are evaluated for each stimulus are:

| LPS | polyI:C | IFNβ | P3CSK4 | CpG | TNF |
|---|---|---|---|---|---|
| Cxcl11 | Socs1 | Socs1 | Slamf8 | Slamf8 | Olr1 |
| Abtb2 | Myo1b | Plac8 | Socs1 | Arrdc4 | Htr2a |
| Tmem200b | Rasgrp1 | Kdr | Nos2 | Glipr2 | Myo1b |
| Arrdc4 | Fgl2 | Il12rb1 | Dusp4 | Olr1 | Rasgrp1 |
| Hopx | Gm4951 | Tnfsf8 | Myo1b | Upp1 | Slamf8 |

In one embodiment, the polarization state of macrophages in the sample are determined. In one embodiment, the macrophages are M0, M1, M2 or any combination thereof. In one embodiment, macrophage types are identified using Irf1, Fg12 and Tgtp1 gene expression. In one embodiment, M0 macrophages are identified by Mx2, Nfkbiz and Egr3 gene expression. In one embodiment, M1 macrophages are identified by Mx2, Tnf and Gna15 gene expression. In one embodiment, M2 macrophages are identified by Cited, Ehd1 and Pou2f2 gene expression.

In one embodiment, the determination of the response specificity score involves an information theoretic algorithm to calculate mutual information or channel capacity. In one embodiment, the determination of the response specificity score involves machine learning algorithm such as ensemble of decision trees, nearest neighbors, or neural nets. In one embodiment, the determination of the response specificity score involves a pre-established relationship between responses of the stimuli-related genes to the stimuli, a pattern of expression among signaling systems, a pattern of expression of gene regulatory systems, a temporal progression of any of the foregoing, or combinations thereof.

In one embodiment, scREAL-TIME analysis provides the response specificity score.

In some embodiments, scREAL-TIME comprises the following components:
1. Dimensionality Reduction: Principal Component Analysis (PCA) is performed on the time-series single-cell RNAseq data. PCA was performed centered and unscaled across all stimuli simultaneously, generating a lower dimensional data embedding that captured the gene expression information of individual cells.
2. k-means clustering: this method assumes that many representative archetypes of cells can estimate the spectrum of behavior of individual cells. Conceptually then, with enough archetypes linked combinatorially across timepoints, the full range of heterogeneous behavior of individual cells can be estimated in a manner that minimizes the heterogeneity arising from technical noise. k-means clustering was utilized on the top 50 PC scores with k=20 to determine archetypes at each time point. At any given measured time-point, a cell may be classified as a member of one of k archetypes. For each archetype at a given time point, a consensus measurement was utilized—an average value for each PC score determined by the members of said archetype from the data.
3. Weighted Random Walks: The probability of archetype connections is labeled across timepoints by the density of cells belonging to that archetype and the Euclidean distance of the mean of each archetype. Upon assigning these probabilities, random walks were generated where each simulation belonged to a particular observed archetype at a given timepoint, adopting the archetype's consensus PC measurements.
4. Spline fitting: For each random walk, interpolation is performed across unmeasured timepoints by fitting polynomial spline interpolants with three degrees of freedom. This linked the lower dimensional cell-level data, resulting in a trajectory for each cell in PC space, with gene expression trajectories for each cell embedded and recoverable from the original PCA loadings.
5. Recovering gene trajectories: The approximately invertible property of PCA was utilized to invert the dimensional reduction transformation using the top 50 PCs, requiring use of the loadings matrix to scale and rotate the lower dimensional cell-based data. This back projected our random walk trajectories from the lower dimensional space to reflect individual gene trajectories for every cell. Each random walk, i.e., each cell, was subsequently represented as a continuous curve in gene expression space.

Calculation of trajectory features. For comparing stimulus-specificity, trajectories were first scaled 0-1 across all stimuli, and the trajectory features were calculated on the scaled trajectories. This was necessary as spline fitting across principal components generated occasional negative values for some cells, especially at the beginning of the trajectory. Scaling maintained the same relative dynamics and amplitudes, and increased the reliability of the trajectory feature calculation. Integral was calculated by integrating each single cell trajectory curve from 0 to 8 hours. Peak amplitude was calculated by taking the maximum value over 8 hours. Max log fold change was calculated for each gene by taking the fold change between the peak amplitude and the same cell's starting value at 0 hours. Activation speed was calculated by taking the derivative of the curve at 1 hour.

Calculation of mutual information. An information theoretic approach was used to identify either individual genes or combinations of genes providing the highest maximum mutual information between ligand identity and gene expression. Error bars on mutual information calculations were done using 10 bootstraps on 50% of the data. Estimation of maximum mutual information was implemented using the R package SLEMI (T. Jetka, K. Nienaltowski, T. Winarski, S. Błoński, M. Komorowski, Information-theoretic analysis of multivariate single-cell signaling responses. PLoS Comput Biol. 15, e1007132 (2019), which uses a statistical learning-based approach to more accurately and more efficiently calculate maximum mutual information for data types with higher dimensional outputs. The max MI was first calculated for each gene individually, using all stimuli (highest theoretical max MI=2.58 bits), as well as for all pairs of stimuli (highest theoretical max MI=1.0 bits).

As will be seen in the description below, the method for determining innate immune system responsiveness as generally described herein can be used to guide therapy for a patient by comparing a patient's RS from a single time point or during the course of monitoring (e.g., regular periodic physical examination) or followed after the diagnosis and/or during the course of treatment of a diseases, to guide selection of therapy (and/or contraindicate others) and monitor recovery. In some embodiments, the methods disclosed herein for obtaining RS, when combined with querying a data bank containing RS data of a plurality of healthy, ill, and ill patients undergoing therapy, the health status and/or effective therapeutic regimen can be identified.

In another embodiment, the present disclosure provides a method for treating a subject having a condition or disease, comprising the steps of: (a) determining the responsiveness of the subject's innate immune system by a method comprising: (i) obtaining a blood sample from the subject; (ii) exposing monocytes or monocyte-derived cells (e.g., macrophages, dendritic cells) derived from the blood samples to one or a combination of stimuli such as cytokines, pathogen-associated molecular patterns, and damage-associated molecular patterns; (iii) determining responses using genome wide RNA profiling, single cell mRNA expression of one or more of stimuli-related genes, or protein expression, in response to the stimuli in the monocytes or monocyte-derived cells (e.g., macrophages, dendritic cells); and (iv) determining a response specificity score characterizing the responsiveness of the subject's innate immune system, wherein the determination comprises use of information theoretic and machine learning methodologies; (b) using the subject's response specificity score to query an information repository comprising (i) innate immune system responsiveness of a plurality of healthy subjects and patients having the condition or disease, and (ii) a correlation between the innate immune system responsiveness and a therapeutic outcome of the patients to one or more therapeutic regimens, and identifying a correlation value for the subject; and (c) treating the subject with a therapeutic regimen when the correlation value indicates a positive therapeutic benefit for the subject; or avoiding treating the subject with a therapeutic regimen when the correlation value indicates an absence of or a negative therapeutic benefit for the subject.

In one embodiment, the blood samples comprise peripheral blood mononuclear cells. In one embodiment, the exposing is to single cells.

In one embodiment, the determining responses is at a single time point after exposing, or at a plurality of time points after exposing. In one embodiment, the plurality of time points includes a zero time point. In one embodiment, the plurality of timepoints are within about 24 hours of exposing. In one embodiment, the plurality of timepoints are within about 12 hours of exposing. In one embodiment, the plurality of timepoints are within about 8 hours of exposing. In one embodiment, the plurality of timepoints are within about 3 hours of exposing. In one embodiment, the exposing after multiple timepoints comprises 0, 0.5, 1, 3, 5 and 8 hours after exposing. In one embodiment, the exposing after multiple timepoints consists of 0, 0.5, 1, 3, 5 and 8 hours after exposing. In one embodiment, the determining the response specificity score is based on a plurality of time points after exposing. In one embodiment, the plurality of timepoints of exposing includes an exposing with no stimuli.

In one embodiment, the determining responses for a plurality of timepoints comprises integral, fold change, peak amplitude, speed, or any combination thereof.

Examples of cytokine stimuli include, but are not limited to, TNF, IFNγ, IL-6, IL-2 or any combination thereof. Examples of pathogen-associated molecular patterns stimuli include, but are not limited to, Pam3CSK, CpG, LPS, polyI:C, flagellin, or any combination thereof. In one embodiment, the pathogen-associated molecular patterns stimuli are from fungi, protozoa, helminths, bacteria, viruses, or any combination thereof. Examples of damage-associated molecular patterns stimuli include, but are not limited to, products derived from lysed cells, necrotic cells or injured cells. In one embodiment, the combinations of stimuli can be IFNγ and IL-6. In one embodiment, the stimuli-related genes can be IkBa, p-RelA, pSTAT1, pSTAT2, pSTAT3, pSTAT5, p-IRF3, p-p38, p-ERK, p-JNK, or any combination thereof.

In one embodiment, the stimuli comprise LPS, polyI:C, IFNβ, P3CSK4, CpG and TNF. In one embodiment, the stimuli consist of LPS, poly I:C, IFNβ, P3CSK4, CpG and TNF.

In one embodiment, a no stimulus control is included. In one embodiment, the stimuli-related genes can be IkBa, p-RelA, pSTAT1, pSTAT2, pSTAT3, pSTAT5, p-IRF3, p-p38, p-ERK, p-JNK, or any combination thereof. Single-cell RNA expression is determined. In one embodiment, antibodies to stimulus-responsive proteins such as Ma, p-RelA, pSTAT1, pSTAT2, pSTAT3, pSTAT5, p-IRF3, p-p38, p-ERK, p-JNK, may be used, singly or any combination thereof.

In one embodiment, the stimuli-related genes comprise Tnf, 116, Cc15, Cc110, Nfkbia, or any combination thereof.

In one embodiment, the top 5 macrophage polarization state related genes which are evaluated for each stimulus are:

| LPS | polyI:C | IFNβ | P3CSK4 | CpG | TNF |
|---|---|---|---|---|---|
| Cxcl11 | Socs1 | Socs1 | Slamf8 | Slamf8 | Olr1 |
| Abtb2 | Myo1b | Plac8 | Socs1 | Arrdc4 | Htr2a |
| Tmem200b | Rasgrp1 | Kdr | Nos2 | Glipr2 | Myo1b |
| Arrdc4 | Fgl2 | Il12rb1 | Dusp4 | Olr1 | Rasgrp1 |
| Hopx | Gm4951 | Tnfsf8 | Myo1b | Upp1 | Slamf8 |

In one embodiment, the polarization state of macrophages in the sample are determined. In one embodiment, the macrophages are M0, M1, M2 or any combination thereof. In one embodiment, macrophage types are identified using Irf1, Fg12 and Tgtp1 gene expression. In one embodiment, M0 macrophages are identified by Mx2, Nfkbiz and Egr3 gene expression. In one embodiment, M1 macrophages are identified by Mx2, Tnf and Gna15 gene expression. In one embodiment, M2 macrophages are identified by Cited, Ehd1 and Pou2f2 gene expression.

In one embodiment, the stimuli-related genes can be obtained by genome-wide RNA profiling. In one embodiment, the stimuli related protein expression can be assessed using antibodies, mass spectroscopy, or other methods. In one embodiment, the determination of the response specificity score involves a machine learning algorithm that uses t-distributed stochastic neighbor embedding (t-SNE). In one embodiment, the determination of the response specificity score involves a pre-established relationship between responses of the stimuli-related genes to the stimuli, a pattern of expression among signaling systems, a pattern of expression of gene regulatory systems, a temporal progression of any of the foregoing, or combinations thereof.

In one embodiment, scREAL-TIME analysis provides the response specificity score. In one embodiment, scREAL-TIME comprises dimensionality reduction, k-means clustering, weighted random walks, spline fitting and recovering gene trajectories. scREAL-TIME is described in detail hereinabove.

In one embodiment, the information repository or data bank used in the above method comprises innate immune system responsiveness of patients at a single or at one or more time points of the condition or disease, for example, at disease diagnosis or onset, at the time when symptoms appear, or during treatment. In one embodiment, the condition or disease can be cancer, inflammatory disease, autoimmunity, high BMI, or advanced age. Other conditions include transplant rejection, tumor progression, tumor immunotherapy, sepsis, or infection.

The terms “comprise”, “comprises”, “comprising”, “includes”, “including”, “having” and their conjugates mean “including but not limited to”.

As used herein, the singular form “a”, “an” and “the” include plural references unless the context clearly dictates otherwise. For example, the term “an enzyme” or “at least one enzyme” may include a plurality of enzymes, including mixtures thereof.

Throughout this application, various embodiments of the present disclosure may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps of the invention and variations thereof are described. This description is not intended to be limiting and changes in the components, sequence of steps, and other variations would be understood to be within the scope of the present invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Each step or component of the assessment disclosed herein is described in further detail below. Such descriptions are merely exemplary of how the step may be carried out; a skilled artisan will recognize variations that will achieve the same purposes. Such variations are fully embraced herein.

Subjects. The disclosure provides for the evaluation of the Responsive Specificity of a subject's or patient's macrophages as an indication of the status of the innate immune system, thus any healthy subject or one having a presumed or overt condition or disease or under a course of acute or chronic therapy may be so evaluated. Moreover, to establish correlations between Response Specificity of a particular subject and the potential benefit, lack of benefit or contraindication to a particular therapy and even duration or intensity of a regimen (e.g., dose level, dosing frequency) thereof (e.g., pharmacologic, life-style modification, etc.), an information repository comprising data on a plurality of subject data on which the correlation between Response Specificity and particular subject's therapeutic history is obtained.

Obtaining blood samples from the subject. The methods described herein are typically carried out using an aliquot of whole blood taken from an antecubital vein, thought the method is not so limiting and a blood sample from an indwelling catheter, sample of blood from a blood donation, or even a finger stick may provide the requisite sample. An anticoagulant such as heparin may be used to prevent clotting. PMBCs may be preserved for future testing by freezing, e.g., in 10% DMSO/90% FBS (fetal bovine serum).

Exposing monocytes, macrophages or other monocyte-derived from the blood samples. In one embodiment, peripheral blood mononuclear cells (PBMCs) are isolated from the blood sample. Methods well known in the art may be employed, such as, by density gradient centrifugation (e.g., using Ficoll), blood collection tube separation components, cell sorting techniques, etc.

Monocytes may be isolated or enriched from PBMCs by methods such as, but not limited to, pan-monocyte positive selection or CD14+ positive-selecting cell sorting (e.g., Miltenyi). Isolated or enriched monocytes may be incubated for a period to allow equilibration, e.g., in complete medium with 1% FBS for 2 hours.

PBMCs (monocytes) may be differentiated into macrophages by any one of a number of methods such as but not limited to using macrophage colony-stimulating factor (M-CSF) alone, or in combination with so-called polarizing cytokines such as IFNγ (M1), IL4 (M2), IL10, or IL13. In one embodiment, monocytes are differentiated into dendritic cells.

Exposure of monocytes or monocyte-derived cells to stimuli. Macrophages are immune sentinel cells that respond specifically to different stimuli. Innate immune macrophages have pattern-recognition receptors capable of sensing pathogens. Many host defense functions may also be detrimental. Macrophages have distinct expression programs activated in response to different immune threats. This disclosure provides for the assessment of the responsiveness of an individual's macrophages (or monocytes or other monocyte-derived cell types) to a panel of stimuli to generate a score (Response Specificity) that is useful for gauging the individual's potential response to innate immune system challenge by a treatment regimen for a particular condition or disease, among other purposes.

Monocytes, macrophages or other monocyte-derived cells obtained as described above are exposed in vitro to individual or combinations of stimuli such as those described further below. In one embodiment, a plurality of similar monocyte or monocyte-derived cell samples in medium are prepared in plates or tubes, and the stimulus/stimuli added (e.g., in a vehicle, carrier, buffer, culture medium, or other suitable solution or suspension). Cells may be incubated at 37° C. in 5% CO2 for a specified time before cells are harvested for subsequent steps. Concentrations of each or combinations of stimuli may be at 1, 5, 25 or 125 mcg/mL (such optimal concentrations for each stimulus or combination to be determined from optimization studies), or where the stimulus is based on a cell extract, for example, may be quantified by protein content, original cell count, or any other assay or quantification process (e.g., to be able to establish a particular stimulus level or amount for consistency across measurements). In one embodiment, an optimal duration of exposure is provided for each stimulus. In one embodiment, a single duration of exposure is provided for all stimuli. In one embodiment a time course of exposure is obtained for one or more stimulus conditions, from sampling at different time points or separate aliquots prepared for harvesting at each time point.

In some embodiments, a trajectory is obtained at a series of time points after exposure, such as over one hour, two hours, three hours, four hours, eight hours, 12 hours, 18 hours or 24 hours after exposure to stimuli. In some embodiments the trajectory is between 0 and 8 hours. In some embodiments the trajectory is between 0 and 3 hours. In some embodiments timepoints include 0, 0.5, 1, 3, 5 and 8 hours. In some embodiments, each stimulus is followed by the same timepoints. In some embodiments each stimulus is followed by a particular series of timepoints that may be the same or different from any other timepoint. In some embodiments, a time course with no stimulus is determined.

In some embodiments, the determining responses for a plurality of timepoints comprises integral, fold change, peak amplitude, speed, or any combination thereof. In some embodiments, integral is calculated by integrating each single cell trajectory curve from 0 to 8 hours. In some embodiments, peak amplitude is calculated by taking the maximum value over 8 hours. In some embodiments, max log fold change is calculated for each gene by taking the fold change between the peak amplitude and the same cell's starting value at 0 hours. In some embodiments, activation speed is calculated by taking the derivative of the curve at 1 hour. These particular assessments are merely exemplary of other selections of timepoints, durations, baseline point, and other factors in providing data for determination of the response specificity score.

As shown in the examples and figures herein, initial assessment of the Response Specificity was evaluated using a larger panel of stimuli, assessing gene expression among a large number of genes, and over a time course of assessment to track the temporal alterations in gene expression after stimulation. For a more readily deployable method to assess Response Specificity for processing patient samples, a subset of stimuli, measured genes, and single time point after stimulation (or specific time point for each stimulus) may be selected to streamline the process while capturing useful data from each individual. However, the disclosure herein is not so limited as to the selection of the stimuli, the genes assessed (as RNA and/or protein), or the use of a single vs. two or more time points, in providing a Response Specificity. The Response Specificity may therefore be determined for a particular combination of the aforementioned parameters, or it may be a composite of two or more assessments (e.g., a single time point assessment and a time-course assessment). In some embodiments, different measures of Response Specificity may be designed for different purposes, such as a static (single stimulus exposure time) or dynamic (two or more time points, such as 0, 0.5, 1, 3, 5, and 8 hours). Such variations in the type of Response Specificity are fully embraced by the disclosure herein.

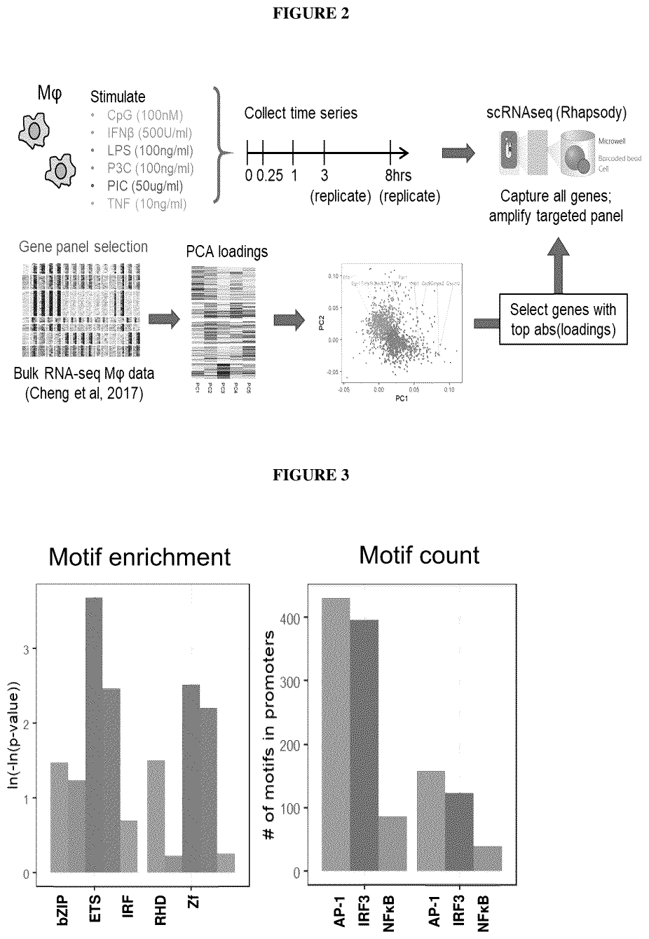

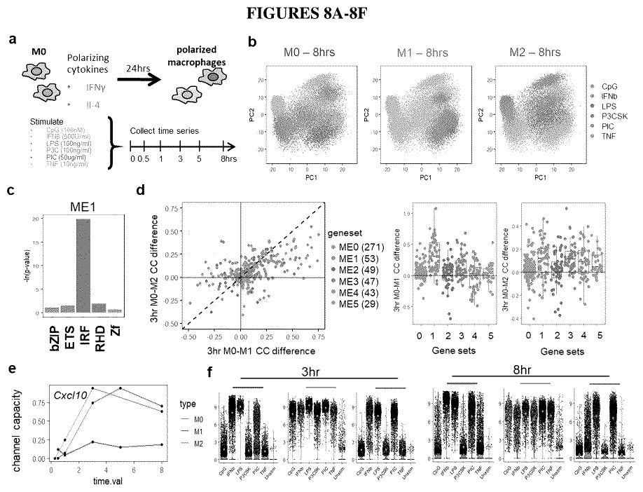

FIG. 2 shows schematic examples of exposing macrophages (including polarized macrophages; see FIG. 8) to various stimuli then assessing single cell gene expression at different time points thereafter. In some embodiments, a single time point after exposure to all stimuli or each stimulus is provided to simplify the cell processing, data collection and assessment.

Cytokines. Cytokines stimuli include but are not limited to TNF, IFNβ, IFNγ, IL-2 and IL6, and any combination thereof. Such combinations include IFNγ and IL6.

Pathogen-associated molecular patterns. Pathogen-associated molecular patterns stimuli (PAMPS) are derived from fungi, protozoa, helminths, bacteria, viruses, or any combination thereof. Non-limiting examples of PAMPS include Pam3CSK, CpG, LPS, polyI:C, flagellin, or any combination thereof.

Damage-associated molecular patterns. Damage-associated molecular patterns (DAMPS) may be derived from lysed cells, necrotic cells, or injured cells. Non-limiting examples of stimuli from such sources include preparations of lysed cells, preparations of or supernatants from necrotic cells, preparations or supernatants from injured cells.

In some embodiments, the combination of LPS, poly I:C, IFNβ, P3CSK4, CpG and TNF are the stimuli. In some embodiments, the stimuli comprise LPS, polyI:C, IFNβ, P3CSK4, CpG and TNF. In some embodiments, the stimuli consist of LPS, poly I:C, IFNβ, P3CSK4, CpG and TNF. In some embodiments, a no stimulus control is included. In some embodiments, no stimulus is evaluated.

Stimuli-related genes. The disclosure is not limited to any particular set of genes or proteins to be profiled. Genes to be profiled in the aforementioned samples including but are not limited to IkBa, p-RelA, pSTAT1, pSTAT2, pSTAT3, pSTAT5, p-IRF3, p-p38, p-ERK and p-JNK. In other embodiments, genes are selected from immune response genes, such as but not limited to Cc13, Cxcl10, Cxcl2, TNF, Irf7, Rgs, Adgre1, I11b, I11a, Lgals9, Cc15, Clec4e, Ddx58, Tnfrsf1b, Cd69, Cc12, Cc14, Cd274, 116, Bc12a1a, Cd14, 1115, Il15ra, Ier3, Ifitm3, Cc19, Lgals1, Ctsd, Hmox1, Btg1, Fth1 and H2.K1, and any combination thereof.

In some embodiments, the stimuli-related genes comprise Tnf, 116, Cc15, Cc110, Nfkbia, or any combination thereof. In some embodiments, the top 5 macrophage polarity stimuli-related genes which are evaluated for each stimulus are:

| LPS | polyI:C | IFNβ | P3CSK4 | CpG | TNF |
|---|---|---|---|---|---|
| Cxcl11 | Socs1 | Socs1 | Slamf8 | Slamf8 | Olr1 |
| Abtb2 | Myo1b | Plac8 | Socs1 | Arrdc4 | Htr2a |
| Tmem200b | Rasgrp1 | Kdr | Nos2 | Glipr2 | Myo1b |
| Arrdc4 | Fgl2 | Il12rb1 | Dusp4 | Olr1 | Rasgrp1 |
| Hopx | Gm4951 | Tnfsf8 | Myo1b | Upp1 | Slamf8 |

In some embodiments, the polarization state of macrophages in the sample are determined. In some embodiments, the macrophages are M0, M1, M2 or any combination thereof. In some embodiments, macrophage types are identified using Irf1, Fg12 and Tgtp1 gene expression. In some embodiments, M0 macrophages are identified by Mx2, Nfkbiz and Egr3 gene expression. In some embodiments, M1 macrophages are identified by Mx2, Tnf and Gna15 gene expression. In some embodiments, M2 macrophages are identified by Cited, Ehd1 and Pou2f2 gene expression.

In one embodiment, the genes are selected from those available on the Rhapsody platform. Selected immune response genes or proteins may be categorized based on their primary known biological function (e.g., gene ontology, such as pro-inflammatory, anti-inflammatory, anti-viral, anti-oxidant, cell death regulator, monocyte attractant, lymphocyte attractant) or on their responsiveness to specific signaling pathways and transcription factors (e.g., such as target genes of NFκB, IRF, AP1 transcription factors).

Determining single cell mRNA expression. After exposure of aliquots of monocytes or monocyte-derived cells (e.g., macrophages, dendritic cells) to the one or more stimuli for a particular time period (that may include zero time or without exposure or exposure to vehicle only, as a control), cells are processed for transcriptomic analysis by, in one embodiment, single cell mRNA expression. In one embodiment, selected immune response genes of single cells in microwells are analyzed by PCR amplification and detected using barcoding.

As described herein, such measurements may be made by, for example, the 10X Genomics platform that profiles expression of all genes in the genome, or the BD Rhapsody scRNAseq platform system, that can profile 500 immune response genes. See, for example, Gao et al., The Comparison of Two Single-cell Sequencing Platforms: BD Rhapsody and 10× Genomics Chromium, Curr Genomics, 2020 December; 21(8):602-609.

Protein expression profiles may also be used to assess Response Specificity. Proteins produced by monocytes or monocyte-derived cells upon stimulation as described herein may be identified by specific assays for such proteins, or methods including but not limited to mass spectroscopic protein identification.

In some embodiments, both RNA and protein expression data are used in the generation of the Response Specificity. In some embodiments the one or more RNA and one or more proteins that comprise the Response Specificity are different. In some embodiments, one or more gene expression measures by both RNA and protein level comprises the Response Specificity analysis.

For initial determination of the optimal selection of stimuli, combination of stimuli, duration of exposure, and immune response related genes to profile, channel capacity will be calculated which comprises evaluation of upstream multi-transcription factor signaling networks and the downstream gene regulatory networks. Channel capacity is calculated for all genes, for single genes, for sets of genes controlled by the same regulatory mechanism, or genes performing different functions (e.g., cytokines, tissue remodelers, feedback regulators).

Once an optimal protocol comprising stimuli and responsive genes, and duration of exposure for each (or a single duration that optimized available of data across the stimuli and genes), the protocol for assessing response specificity in subject and patient samples is established, and a single or set of numerical values can be ascribed to each monocyte/macrophage sample based on such analysis. An information repository comprising such data is prepared, together with each subject's or patient's health status, any treatments thereof and outcomes thereof.

Determining single cell protein expression. Labeled antibodies, antigen-binding fragments or receptor reagents to expressed proteins may be used to identify stimuli-induced responses in the monocytes or monocyte-derived cells. In another embodiment, mass spectroscopy is used to identify proteins expressed by stimulated cells. As with the RNA expression methods described above, the disclosure is not limited to any particular method of quantifying gene expression, and embraces any and all such methods. In some embodiments, gene expression may be measured by both RNA and protein expression for individual genes.

Determining a response specificity score. As described above, the response specificity score is a value or values characterizing the innate immune system responsiveness of the subject, based on the testing of monocytes or monocyte-derived cells such as macrophages and dendritic cells, as described herein. The determination comprises use of information theoretic and machine learning methodologies. In one embodiment, the determination of the response specificity score involves a machine learning algorithm that uses t-distributed stochastic neighbor embedding (t-SNE). In one embodiment, the determination of the response specificity score involves a pre-established relationship between responses of the stimuli-related genes (and/or proteins) to the stimuli, a pattern of expression among signaling systems, a pattern of expression of gene regulatory systems, a temporal progression of any of the foregoing, or combinations thereof. In one embodiment, the method comprises scREAL-TIME, as described herein.

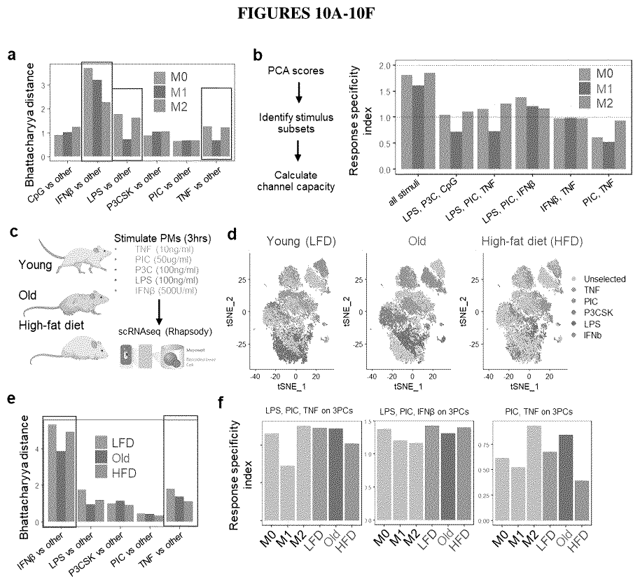

FIG. 10F depicts a Response Specificity Index. In one embodiment, the model is developed by analyzing gene expression patterns over time after stimulation of macrophages with the stimuli described herein. High dimensional data may be visualized using t-distributed stochastic neighbor embedding (tSNE). Machine learning using the nearest neighbor algorithm identifies which stimulus conditions are optimal for generating the Response Specificity.

In another embodiment, scREAL-TIME is used to analyze the stimuli data generated as described above. scREAL-TIME comprises the following components:
1. Dimensionality Reduction: Principal Component Analysis (PCA) is performed on the time-series single-cell RNAseq data. PCA was performed centered and unscaled across all stimuli simultaneously, generating a lower dimensional data embedding that captured the gene expression information of individual cells.
2. k-means clustering: this method assumes that many representative archetypes of cells can estimate the spectrum of behavior of individual cells. Conceptually then, with enough archetypes linked combinatorially across timepoints, the full range of heterogeneous behavior of individual cells can be estimated in a manner that minimizes the heterogeneity arising from technical noise. k-means clustering was utilized on the top 50 PC scores with k=20 to determine archetypes at each time point. At any given measured time-point, a cell may be classified as a member of one of k archetypes. For each archetype at a given time point, a consensus measurement was utilized—an average value for each PC score determined by the members of said archetype from the data.
3. Weighted Random Walks: The probability of archetype connections is labeled across timepoints by the density of cells belonging to that archetype and the Euclidean distance of the mean of each archetype. Upon assigning these probabilities, random walks were generated where each simulation belonged to a particular observed archetype at a given timepoint, adopting the archetype's consensus PC measurements.
4. Spline fitting: For each random walk, interpolation is performed across unmeasured timepoints by fitting polynomial spline interpolants with three degrees of freedom. This linked the lower dimensional cell-level data, resulting in a trajectory for each cell in PC space, with gene expression trajectories for each cell embedded and recoverable from the original PCA loadings.
5. Recovering gene trajectories: The approximately invertible property of PCA was utilized to invert the dimensional reduction transformation using the top 50 PCs, requiring use of the loadings matrix to scale and rotate the lower dimensional cell-based data. This back projected our random walk trajectories from the lower dimensional space to reflect individual gene trajectories for every cell. Each random walk, i.e., each cell, was subsequently represented as a continuous curve in gene expression space.

Calculation of trajectory features. For comparing stimulus-specificity, trajectories were first scaled 0-1 across all stimuli, and the trajectory features were calculated on the scaled trajectories. This was necessary as spline fitting across principal components generated occasional negative values for some cells, especially at the beginning of the trajectory. Scaling maintained the same relative dynamics and amplitudes, and increased the reliability of the trajectory feature calculation. Integral was calculated by integrating each single cell trajectory curve from 0 to 8 hours. Peak amplitude was calculated by taking the maximum value over 8 hours. Max log fold change was calculated for each gene by taking the fold change between the peak amplitude and the same cell's starting value at 0 hours. Activation speed was calculated by taking the derivative of the curve at 1 hour.

Calculation of mutual information. An information theoretic approach was used to identify either individual genes or combinations of genes providing the highest maximum mutual information between ligand identity and gene expression. Error bars on mutual information calculations were done using 10 bootstraps on 50% of the data. Estimation of maximum mutual information was implemented using the R package SLEMI (T. Jetka, K. Nienaltowski, T. Winarski, S. Błoński, M. Komorowski, Information-theoretic analysis of multivariate single-cell signaling responses. PLoS Comput Biol. 15, e1007132 (2019), which uses a statistical learning-based approach to more accurately and more efficiently calculate maximum mutual information for data types with higher dimensional outputs. The max MI was first calculated for each gene individually, using all stimuli (highest theoretical max MI=2.58 bits), as well as for all pairs of stimuli (highest theoretical max MI=1.0 bits).

Various embodiments and aspects of the present invention as delineated hereinabove and as claimed in the claims section below find experimental support in the following examples.

### Example 1

### Response Specificity of Macrophages Sensing Immune Threats

This and the ensuing examples focus on developing ways to measure Response Specificity of macrophages in response to various stimuli. By quantifying this property, one can ascribe a score to macrophages derived from different physiological or pathological conditions and identify its molecular drivers to address both fundamental biological questions and advance clinical studies, including the establishment of normal score ranges and detection of changes due to disease. The importance of innate immune health in guarding against many diseases suggests that Response Specificity could advance to be a useful clinical measurement, prescribed to people, for example, with or at risk of inflammatory disease.

As macrophages function as single cells, their Response Specificity must be measured at the single cell level. This is challenging because (i) biological responses show a high degree of cell-to-cell variability and dynamically evolve over hours, and (ii) single cell measurements of cellular transcriptomes (e.g., single cell RNA-seq) typically have high technical uncertainty and are snapshot time point assays because it is a destructive technology.

These challenges to quantify Response Specificity are addressed in a two-step approach. First, a new experimental workflow is used to track the activity of a key immune response related genes (e.g., transcription factor NFκB, used in this example) at single cell resolution in hundreds of macrophages over time. NFκB is dynamically regulated and its temporal trajectories show a high degree of stimulus-specificity. These high-quality trajectory datasets can be used to develop powerful information theoretic and machine learning approaches to quantify NFκB Response Specificity, and construct mathematical modeling to identify determinants of specificity and confusion. Expansion of the method to encompass a panel of stimuli and a larger set of responsive genes that together, distinguish among innate immune responsiveness across a population of healthy and non-healthy individuals (of e.g., different ages, body compositions, lifestyles, health status, acute disease, chronic disease, duration or stage of disease, etc.) to provide a composite value or score. Such value, as described herein, has valuable diagnostic, prognostic and therapeutic utility. It should be noted that although NFκB and in some cases bone-marrow derived macrophages are used in the initial design of the methodology for determining Response Selectivity, later examples herein provide the experimental basis for a method using peripheral blood derived mononuclear cells, a panel of stimuli to which samples of such cells are exposed (including after differentiation), and analysis of expression of genes by single cells to provide the data input to determine Response Selectivity.

### Conceptual Innovation

The conceptual innovation is that hallmark of innate immune cells is Response Specificity. Response Specificity could be developed into clinical measure of innate immune health that could benefit people with pre-existing conditions that put them at risk for, e.g., infections or autoimmunity, as well as guide treatment and selection of the drug(s), dose, timing, and monitoring of effectiveness. Response Specificity can be measured by the following Experimental and Analytical Innovations.

### Experimental Innovation

A new knock-in mVenus-RelA reporter mouse has been developed that allows tracking of NFκB dynamics in primary cells without the artifact of ectopic expression or immortal cell lines. An optimized workflow is also developed for long term live cell microscopy, including automated image analysis.

A new experimental workflow has been developed (e.g., with the BD Rhapsody system) for single cell profiling the expression of 500 immune response genes selected to capture the diversity of responses in bulk RNA-seq experiments. The Rhapsody single cell platform produces more quantitatively reliable data and is substantially more cost-effective for 10,000's of cells than other approaches (Table 1). However, the disclosure herein is not so limited to any particular method for scRNA sequencing.

TABLE 1
Comparison of single cell sequencing methods

| | 10x genomics | Fluidigm C1 | sc-ddPCR | Rhapsody |
|---|---|---|---|---|
| Method type | droplet | Microwell | Emulsion | Microwell |
| | | chip | droplets | chip |
| Dropout % | 93% [ref. | 45% [ref. | 0% [ref. | ~40-60% |
| | 12] | 13] | 14] | |
| # of genes | ~20,000/cell | ~20,000/cell | a few | 500/cell |
| | | | | (panel) |
| Throughput | ~10,000s cells | ~100-1000s | ~100s | ~10,000s |
| Cost/sample | High: $1000s | Mid: $100s | Low: $50s | Mid: $100s |

### Analytical Innovation

(1) Machine Learning classification to detect confusion. Machine learning approaches that provide measures of specificity and precision, and, importantly, detect conditions of confusion (off-diagonal) have been developed.

(2) Dynamic Mutual Information (dMI). A Markov model-enabled method is developed to quantify the information present in dynamic trajectories and information accumulation over time.

(3) Modeling to better characterize biological variability. Mechanistic modeling approaches that leverage knowledge of biochemical regulatory mechanisms to better characterize single cell responses are developed. A key limitation of single cell measurements is technical noise, which is difficult to separate from biological variability. While smoothing functions distort many of the informative features in gene expression trajectories, model simulations may preserve these features and examine whether they can account for biological heterogeneity. Furthermore, a key limitation of scRNA-seq as a snapshot assay can be overcome by leveraging knowledge of a gene regulatory mechanism in kinetic models that are fit to the data.

### Example 2

### Context-Dependence of Transcriptomic Response Specificity in Macrophages

The downstream result of Response Specificity in signaling networks is the transcription of genes encoding potent inflammatory molecules, tissue remodelers, or cell death initiators. The production of these molecules can be harmful to the host. For example, the hyper-inflammatory cytokine storms recently seen in COVID-19 may be attributed to loss of specificity in the transcriptomes macrophage must produce to effectively control the infection without collateral damage. Environmental context and prior exposures, either pre-existing conditions in people susceptible to infectious disease or the effect of vaccination, may be an additional important factor in the development of severe or mild symptoms. Measuring and quantifying transcriptomic Response Specificity across different environmental contexts will identify genes and regulatory processes that control this property of macrophage function. Thus, the Response Specificity is affected by the health of the PBMC donor.

### An Experimental Framework for Measuring Transcriptomic Response Specificity

Macrophages each function independently as single cells, rather than as an organ, to respond to the environment. Thus, the accuracy of each macrophage's transcriptomic response to the danger signal encountered is vital to averting damaging run-away inflammation. Here, a single cell RNA-seq (scRNAseq) experimental workflow is established to measure Response Specificity.

By stimulating macrophages with diverse immune ligands and obtaining single cell transcriptomes using the BD Rhapsody single cell platform, one can measure the different transcriptomic responses of macrophage cells to various stimuli over a time-series. In preliminary results, 31 samples (5 time points×6 stimuli, plus unstimulated) on naïve M0 macrophages were collected and it was observed that transcriptomes were indeed stimulus-specific, with each ligand producing a population of cells transcriptomically distinct. To measure how prior inflammatory contexts perturb Response Specificity, one can polarize macrophages using IFNβ, IL-4, IL-13, IL-10, or IFNβ to produce macrophages of different epigenetic states, which are thought to be either pro- (M1 associated states) or anti-inflammatory (M2 associated states). One can also stimulate the macrophages with a set of diverse ligands targeting both pathogen receptors and cytokine receptors and collect single cell data over a time-series (FIG. 2). Examples of stimuli can include 1) cytokines targeting two major cytokine receptors: TNF and IFNβ, 2) bacterial ligands: LPS representing Gram negative bacteria, P3CSK representing Gram positive bacteria, and CpG and flagellin, bacterial components, 3) ligands stimulating anti-viral responses, p(I:C) targeting TLR3 and R848 targeting TLR7/8. The experimental workflow would allow measurement of Response Specificity in various cytokine milieus.

These experiments could deliver a well-controlled map of physiological context-dependent macrophage transcriptomes and their time evolution, onto which macrophage responses in disease contexts could be compared against.

### Quantify Response Specificity Using Classification and Information Theoretic Analysis

There has yet to be a quantification of transcriptomic Response Specificity, a key property of macrophage function. scRNA-seq data enable this calculation, and also provides insight into the genes that contribute most to alterations in Response Specificity in physiological and pathological inflammatory contexts. Because use of machine learning models and information theoretic calculations have been shown to be amenable to analysis of high dimensional single cell data as discussed above, one can leverage these analyses to analyze the scRNA-seq data to provide quantitative values to assess Response Specificity. Classification and mutual information would enable quantitative comparison of Response Specificity in different inflammatory contexts and point to genes important for ligand discrimination.

Machine Learning Approach: To quantify the specificity of macrophage gene expression profiles at each of the 5 time points, one can train machine learning classification algorithms on the scRNA-seq data. One could use an ensemble learning approach, training models on a set of machine learning algorithms of different classes such as: decision trees, k-Nearest Neighbors, linear and radial kernel Support Vector Machines, and Naïve Bayes, to avoid computational biases caused any single machine learning algorithm. Machine learning models can be trained by 10-fold cross-validation on 70% of the single cell data to minimize overfitting. Classification accuracy, precision, and F1 scores can be calculated on the remaining 30% of the data, for the 8 stimuli measured in experiments.

Information Theoretic Approach: As a second metric for quantifying specificity, one can calculate the channel capacity using existing methods that describes the upper bound on the ligand discriminability measured in macrophage transcriptomes. The channel in this scenario includes both the multi-transcription factor signaling network, and the gene regulatory network induced by the immune ligand. Channel capacity can be calculated for all genes, single genes, sets of genes controlled by the same regulatory mechanism, and classes of genes performing different functions (i.e., cytokines, tissue remodelers, feedback regulators, etc.).

It is expected that later time points will contain more stimulus-specific information, but for certain pairs of stimuli, earlier time points will display greater ligand discriminability, pointing to the contribution of temporal regulation in defining stimulus-specificity. From the machine learning models one can extract genes that best classify particular ligands at each time point. Channel capacity calculations could allow insight into how different subsets of inflammatory gene programs contribute to ligand distinguishability. Comparing naïve M0, M1-IFNγ, M1-IFNβ, M2a-II4, M2a-II13, and M2c-II10 macrophages using classification and channel capacity metrics could also ultimately reveal the context-dependent importance of specific genes for distinguishing stimuli.

### Reconstruct Response Trajectories for Improved Dmi and Classification

Macrophages activate stimulus-specific sets of genes that also need to be produced with proper timing. However, because scRNA-seq measurements are endpoint and high-throughput transcriptional data of endogenous genes is snapshot in nature, thus far in the field tracing the dynamics of gene expression for single cells has been an outstanding problem. Although single cell expression trajectories have been captured for a few genes at a time with reporter constructs, technological limitations prevent the same for hundreds of endogenous genes. Thus, an analytical framework is needed to model gene expression trajectories that faithfully capture observed distributions in scRNA-seq data. A more accurate analysis of the heterogeneity of induced inflammatory gene programs is described herein. Single cell expression trajectories would allow a better quantification of response specificity. Once developed, expression at single time points for each gene or, ideally for all genes measured, would simplify the workflow to obtain Response Specificity on PBMC samples. Such data collection at a single time point is embodied herein.

To better understand the temporal component of response specificity, a new computational framework is developed to analyze heterogeneous response trajectories using mathematical models, which can be used to investigate the sources and mechanisms behind gene expression variation. Starting from time-series, one could stimulate each gene's gene regulatory mechanism (GRM) with the ODE models. One can use stimulus-specific transcription factor activity curves as input into the model. For each gene, one can scan the stimulus-specific expression trajectories for all plausible gene regulatory mechanisms that involve the previously described combinatorial and temporal action of 4 effector proteins that control either transcription or mRNA half-life. One could first fit simulated trajectories to the data mean using Levenberg-Marquardt least-squares fitting or gradient descent. Then one could use Markov Chain Monte Carlo sampling to generate parameter distributions that result in trajectories fitted to the single cell data distribution. With single cell trajectories in hand, one could calculate dMI and classification metrics on the trajectories as was done for NFκB signaling data described above. Unlike the multiple algorithms already developed for signaling networks, to reconstruct the gene regulatory network of the single cells one could bin the trajectories for each gene and assume a number of cell archetypes to group trajectories of different genes into a single cell, constrained by data-derived gene-gene correlations.

In one embodiment, trajectory information could improve the quantification of the true Response Specificity by incorporating the temporal component. The mechanistic modeling approach described herein would allow a framework to learn how gene regulatory mechanisms control heterogeneity of inflammatory genes (e.g., genes controlled by NFκB “OR” IRF may have more variable expression across cells, while genes controlled by ‘AND” gate activity of multiple TFs may have less expression heterogeneity). Both dMI and classification would point to features of temporal control of gene expression, including speed of gene activation, or fold change of gene expression, essential features of appropriate deployment of innate immune gene programs. However, the invention is not so limiting as to a single time point of assessment or a plurality of timepoints for generating the Response Specificity.

### Example 3

### Macrophage Response Specificity in Inflammatory Disease

The health of the innate immune system is a key determinant of an individual's susceptibility to infectious agents and auto-inflammatory disease. Response Specificity, as a fundamental functional property of innate immune cells, may serve as the thermometer to measure Innate Immune Health. Experimental and analytical workflows have been described above to measure the Response Specificity of macrophages via two single cell assays: either live-cell microscopy of gene expression activity, or scRNA-seq to characterize how Response Specificity may be affected by specific polarizing cytokines that, in vivo, define the tissue microenvironment. This example examines how macrophage Response Specificity may be affected by inflammatory disease. First, mouse models of inflammatory disease are examined, e.g., use the mVenus-RelA reporter to measure Response Specificity as well as transcriptomic Response Specificity. Then transcriptomic Response Specificity of monocyte-derived macrophages from human donors (e.g., healthy, with autoimmune disease, or at risk of autoimmune disease due to receiving checkpoint inhibitors for tumor-immunotherapy) are measured. The goal for these studies is to enable eventual definition of healthy vs. unhealthy ranges of Response Specificity scores (e.g., MI) for a given set of stimuli, with the vision that Response Specificity could be commonly measured in the clinic for people at risk for inflammatory disease conditions, similar to CBC panels, glucose-tolerance tests, or tests for liver and kidney enzymes. What score may be associated with auto-immune risk, and further determination of which genes, regulatory regions, or stimuli are most reliably informative would inform future translational studies in developing a cost-effective, targeted test of Response Specificity that can be offered in the clinic as a measure of Innate Immune Health.

### Macrophage Response Specificity in Mouse Models of Inflammatory Disease

This example describes applying the Response Specificity workflow to macrophages derived from mouse models of auto-immune or inflammatory disease to determine how these diseases may be reflected in Response Specificity. This is a basis for subsequent human donor studies.

In one embodiment, three genetic mouse models of auto-immune and inflammatory disease can be used. Macrophages responses may be altered in a cell-autonomous manner or through an altered cytokine milieu produced by other cells. Hence, not only bone-marrow-derived macrophages, but also peritoneal macrophages that have been conditioned in vivo are studied. The workflow described above can be applied:
1. Breed mouse model strain into the mVenus-RelA reporter line to enable Response studies, in this example, NFκB.
2. Live cell imaging of NFκB responses in bone-marrow-derived macrophages (BMDMs) and peritoneal macrophages for 8 immune threats.
3. BD Rhapsody workflow for transcriptomic responses in the optimized time course to 8 immune threats.
4. Information Theoretic and Machine Learning analysis to quantify Response Specificity, and identify features, genes, gene sets, and ligands that are most effective to distinguish healthy from pathological states.

Model 1: Sjögren's syndrome (SS) is a systemic autoimmune disorder involving progressive destruction of tissues exposed to the environment, such as eye, mouth and throat, and skin rashes. Interestingly, several genetic variants in regulators of the inflammatory transcription factor NFκB are associated with SS patients and a mouse strain containing similar variants recapitulates some of the SS pathognomonic characteristics. Preliminary results reveal a diminished NFκB Response Specificity in BMDMs sensing cytokine TNF, the bacterial PAMP LPS, and the viral PAMP poly(I:C), and that information accumulation was diminished during the 0.75-2 hour phase, which coincides with the function of the IκBα negative feedback loop that is disrupted in this mouse model.

Model 2: C57Bl/6 AireGW/+ mice develop Sjögren's syndrome and autoimmune retinopathy. This mouse is a model for human patients with the G228W dominant-negative mutations in AIRE, who are predisposed to endocrine autoimmunity. These mice are protected from autoimmunity with early-life injections with anti-TNF. AIRE normally promotes self-tolerance by participating in negative selection of self-reactive T-cells. In an ex vivo model of human macrophages, loss of AIRE increased susceptibility to Candida albicans infection, which was associated with failures in secretion of IL-1β, IL-6, or TNF. Thus, macrophage Response Specificity may be affected, but via cytokine conditioning rather than cell-autonomously.

Model 3: Absence of tonic TNF conditioning. Anti-TNF therapy is widely prescribed but is associated with paradoxical inflammatory symptoms, such as psoriasis. Preliminary studies with macrophages deficient in tonic TNF indicate that both NFκB and transcriptomic responses are less specific, as responses to TNF and Pam3CSK are more similar than in healthy controls.

With these studies, it is expected to find that macrophage Response Specificity is diminished in mouse models of auto-immune or auto-inflammatory disease and that specific gene sets and ligands would be most informative about such abnormal function.

Response Specificity of Macrophages from Humans at Risk for Inflammatory Disease

Following studies in the above mouse model systems, one can apply the optimized workflow described herein for quantifying transcriptomic Response Specificity in macrophages from human donors. One underexplored but increasingly prevalent cause of autoimmunity are patients who have been treated with anti-tumor checkpoint-blockade drugs, a fraction of whom are at risk for developing autoimmune syndromes. PD-1 and PD-L1, the receptors and ligand inhibited by checkpoint blockade, are essential for immune tolerance. PD-1 and PD-L1 are expressed on multiple cell types, including T-cells directly targeted in immunotherapy but also on macrophages. In mice, knockout of these genes lead to lupus-like syndromes, type 1 diabetes, or arthritis. In humans, the effect of PD-1/PD-L1 inhibition on innate immune function is not well understood. In both mice and humans, checkpoint inhibitor-induced autoimmune symptoms can be ameliorated with TNF blockade, suggesting that overproduction of key macrophage-response cytokines may be a contributor to loss of self-tolerance.

In one embodiment, one could measure Response Specificity of PBMC-derived macrophages in human cohorts that develop autoimmunity as a result of treatment with checkpoint inhibitors intended to block PD-L1/PD-1 on T-cells. To include appropriate controls, one could measure 4 donor groups: (1) without autoimmunity, not on checkpoint blockade (healthy); (2) with autoimmunity, not on checkpoint blockade therapy; (3) without autoimmunity, on checkpoint blockade; and (4) with autoimmunity and on checkpoint blockade therapy. One could isolate monocytes from PBMCs, culture macrophages ex vivo, stimulate them with the 8 immune ligands described above, and collect single cell transcriptomes using the Rhapsody scRNAseq workflow described above. The data can be analyzed using the classification and information theoretic algorithms described herein.

It is anticipated that compared to Group 1 healthy controls, both Group 2 and 4 patients would have decreased Response Specificity, and the ligands that are confused as well as the identified gene programs contributing to mis-regulation would guide refinements and downscaling of the assay. Comparison of Group 3 macrophages would control for the effect of tumor-conditioning on macrophage Response Specificity.

### Example 4

### Macrophages are Immune Sentinel Cells that Respond Stimulus-Specifically

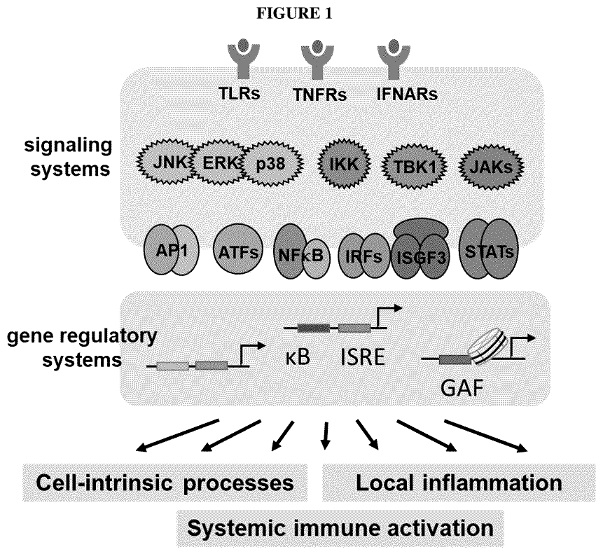

FIG. 1 shows that macrophages are immune sentinel cells that respond stimulus-specifically. Innate immune macrophages have pattern-recognition receptors and cytokine receptors capable of sensing pathogens stimulus-specifically. As sentinel cells, they response to thousands of possible pathogen or damage-related immune threats. Many host defense functions may also be detrimental to the host, and it is thus advantageous that they be deployed carefully.

### Example 5

### Quantifying Stimulus-Response Specificity

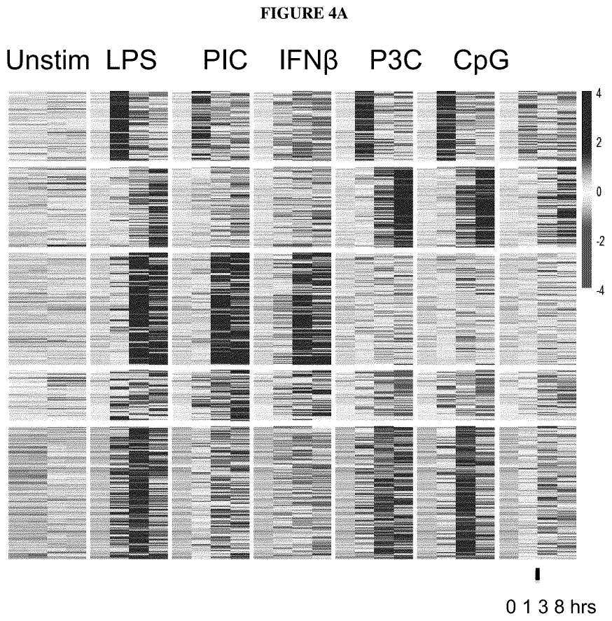

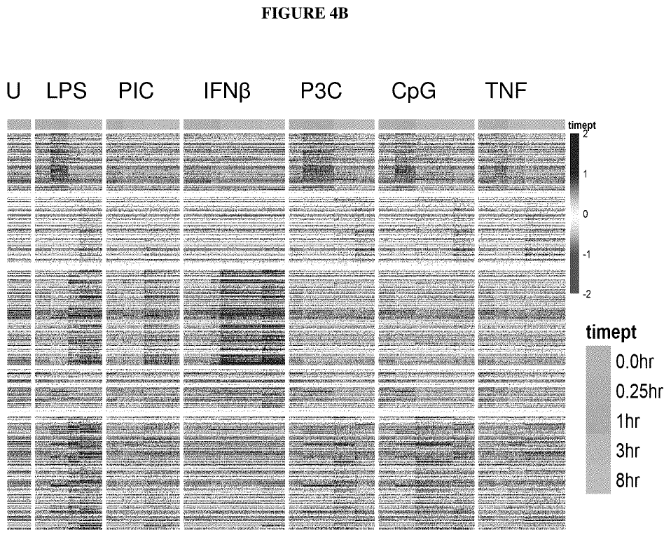

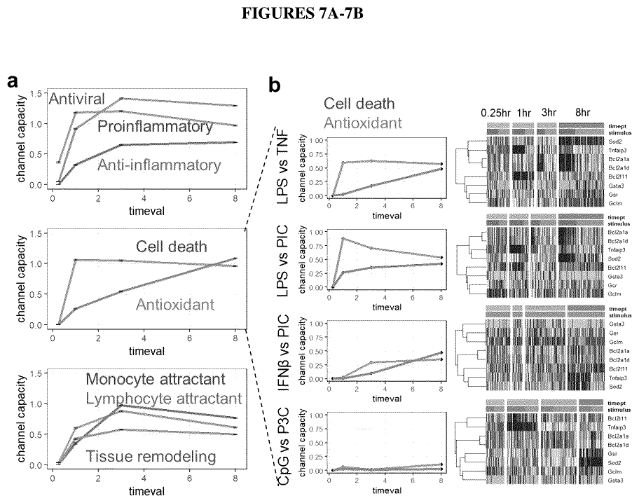

FIG. 2 (with FIGS. 3 and 4A-B) depicts the approach disclosed herein for quantifying stimulus-response specificity. FIG. 7 shows macrophages exposed to a variety of immune ligands representing Gram-negative or Gram-positive bacteria, viral nucleic acid, or host cytokines. Single cell RNAseq data is collected over time points using the Rhapsody system, and a set of 500 immune response genes are sequenced. As noted herein, for determining Response Specificity, a single time point of exposure for all stimuli, or a single time point optimized for each stimulus, may be employed.

The optimal selection of genes to comprise a panel for routine determination of Response Specificity is identified by analysis of heatmaps of bulk RNA sequencing data, conducting principal component analysis (PCA), and identifying the target panel therefrom.

FIG. 3 depicts motif enrichment of all induced genes vs gene selected for the Rhapsody panel shows further enrichment of AN, NFκB and IRF pathway target genes.

FIGS. 4A and 4B show relative patterns of expression recapitulated between published bulk data and single cell data.

### Example 6

### Expression Variances are Gene-, Stimulus- and Timepoint Specific

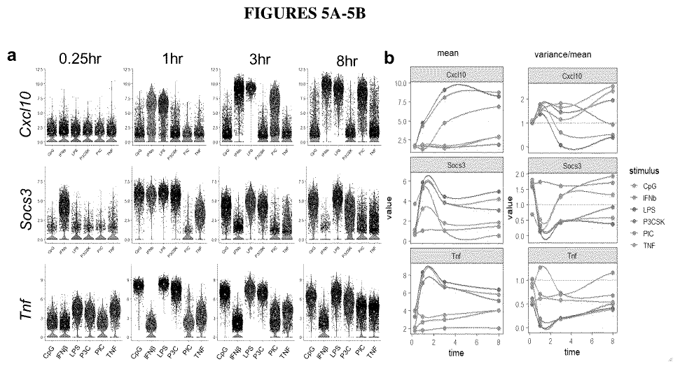

FIG. 5 shows that expression variances are gene-, stimulus-, and timepoint-specific. A) Three examples of cytokine or feedback regulator genes at multiple timepoints. Green points represent the mean. b) Quantification of mean and Fano factor for each stimuli for the three genes. Increases in Fano factor indicate greater variability for that timepoint.

### Example 7

### Gene Expression Heterogeneity Hampers Response Specificity

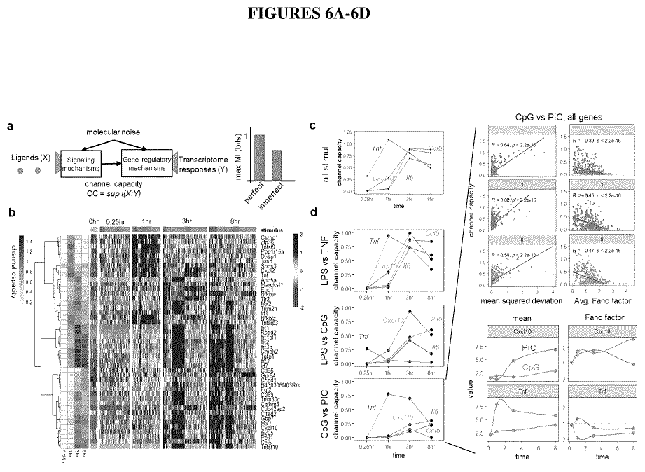

FIG. 6 shows that gene expression heterogeneity hampers response specificity. a) Channel capacity (CC) schematic. b) CC of individual genes with heatmap of expression. c) Examples from b). d) CC of pairwise stimulus comparisons: bacterial vs cytokine, bacterial vs bacterial, bacterial vs viral. For specificity of CpG vs pIC, a zoom-in of the mean and variance contribution to response specificity. Response specificity of Cxcl10 is low due to high heterogeneity, even though mean expression values are distinct.

### Example 8

### Different Biological Functions Show Distinct Response Specificities

FIG. 7 shows that different biological functions show distinct response specificities. a) Cell intrinsic antiviral (Ifit3, Mx1, Mx2, Oasl1) and systemic immune activating pro-inflammatory (Tnf, 116, I11a, I11b) categories both have the highest stimulus-specificity. b) CC of Cell death (Bc12a1a, Bcl2ald, Bcl2111, Tnfaip3) and Antioxidant genes (Gsr, Sod2, Gc1m, Gsta3) for pairwise stimuli comparisons. Low expression variance of cell death genes during LPS stimulation results in high CC for LPS vs host or viral ligands.

### Example 9

### M1 and M2 Polarization Affect Response Specificity Differently

FIG. 8 shows that M1 and M2 polarization affect response specificity differently. a) Data collection schematic. b) PCA of M0, M1, M2 macrophage responses at 8 hrs. c) Motif enrichment of WGCNA modules; ME1 is an IRF module. d) Individual genes in the interferon pathway (ME1 by WGCNA), lose specificity in M1 macrophages. e) CC for example gene Cxcl10 in M0, M1, M2 over time. f) Violins for Cxcl10 in M0, M1, M2.

### Example 10

### The Genes Most Informative of Response Specificity are not Cytokines

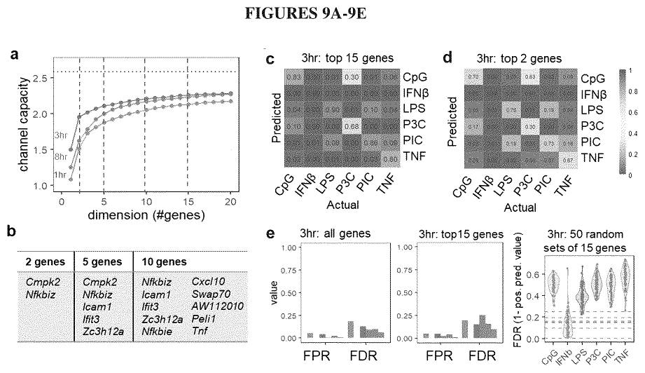

FIG. 9 depicts the genes most informative of response specificity are not cytokines. a) CC of the best possible combinations of n genes, up to 20 dimensions. At 3 and 8 hrs, 5 genes already exceeds 2 bits, corresponding to a capacity to distinguish 4 stimuli. b) Best combination of genes for 2, 5, 10 dimensions. c) Random forest classifier to predict stimuli, using either all 15 top genes or the top 2 genes identified by channel capacity analysis. FIG. 9E shows FDR using a subset of top 15 genes (middle panel) is comparable to all genes (left panel) and better than random sets of 15 genes (right panel).

### Example 11

### Response Specificity Index to Assess the Health of Innate Immune Cells

FIG. 10 describes a Response Specificity Index to assess the health of innate immune cells and a scoring method for response specificity. a) Bhattacharyya distance between each stimulus and all others for M0, M1, M2 to identify informative stimulus subsets. b) Channel capacity calculated on the first 3 PCs of M0, M1, M2 macrophages forms the Index. c) Applying the Index to young (16 weeks), old (>90 weeks), or obese (16 weeks) mice peritoneal macrophages (PM). d) tSNE of PM responses. e) Distance metric for PMs after projection onto M0, M1, M2 PCA. f) More M2-like IFNβ and TNF response in old PMs. Decreased response specificity in PMs from high-fat diet obese mice due to TNF & pIC mixing.

### Example 12

### Profiling Signaling Networks and Functionality of Innate Immune Cells in Breast Cancer Patients

This example describes combining high-dimensional flow cytometry and single cell RNA sequencing (scRNAseq) with information theory (IT) to more completely characterize signaling networks and functionality in innate immune cells in breast cancer patients as compared to healthy controls.

To enhance our understanding of the immune system immediate response, innate immune cells from peripheral blood samples taken from breast cancer patients are studied. Flow cytometry-based profiling of the signaling network and scRNAseq profiling of the resulting transcriptome are used to identify biomarkers that are prognostic and useful in informing therapeutic options. Information theory algorithms are used to analyze high dimensional data that includes many nonlinear interactions in order to establish clinically informative scores for how the functional health of innate immune cells are altered in breast cancer patients, at the level of both cellular signaling and gene expression.

Workflow for Flow Cytometry. Blood samples from healthy donors are collected at 0, 1, and 6 months to provide three independent time points. Blood samples are processed into PBMCs and preserved. Banked frozen PBMCs are thawed and analyzed in batches to minimize variability. To measure the effects of cytokines (e.g., TNF, IFNγ, IL6) and PAMPs (e.g., CpG, LPS, P3CSK, and polyI:C) stimulation on the monocytes isolated from PBMCs, individual stimuli are used alone or in combination at 15, 30, and 60 min after stimulus application.

To investigate whether IFN-γ and IL-6 interact with each other in the same cells, one can simultaneously stimulate PBMCs with IFN-γ and IL-6 at varying combinations of concentrations (e.g., 1, 5, 25, and 125 ng/ml) and measured the effects of IL-6 on IFN-γ-induced pSTAT1 AMFI. Representative analytes include, but are not limited to, IkBa, p-RelA, pSTAT1, pSTAT2, pSTAT3, pSTAT5, p-IRF3, p-p38, p-ERK, and p-JNK. Possible study time points can be 10′, 30′, 60′, 120′, and 240′.

Workflow for scRNAseq (BD Rhapsody) Profiling of Monocyte Responses. To establish an efficient transcriptomic assay to probe the health of PBMC-isolated monocytes, one can freeze PBMCs from healthy donors in 90% FBS/10% DMSO. Monocytes can be selected using either pan-monocyte negative-selection or CD14+ positive-selection MACS sorting, which typically yields 15-20% monocyte cells from healthy PBMCs. After allowing the monocytes to equilibrate in complete media with 1%1-BS for 2 hrs, one can stimulate with 8 cytokine or PAMP ligands (e.g., TNF, IFNβ, IFNγ, IL6, CpG, LPS, P3CSK, polyI:C) at 2 doses, collecting at 4 and 10 hrs time points for scRNAseq. The BD Rhapsody scRNAseq platform can profile 500 immune response genes, which are selected to be genes with maximum response diversity from bulk data.

Channel capacity can be calculated to quantify single cell transcriptomic responses across stimuli at each time point and determine which stimuli are most informative. Using existing methods, channel capacity describes the upper bound on the ligand discriminability. With scRNAseq data, the channel includes both the upstream multi-transcription factor signaling network and the downstream gene regulatory network. Channel capacity can be calculated for all genes, single genes, sets of genes controlled by the same regulatory mechanism, or genes performing different functions (e.g., cytokines, tissue remodelers, feedback regulators, etc.). The overall information capacity for different sets of ligands and doses can be compared. Completion of these studies would allow one to select 4 ligands at the most informative doses, as well as 1 time point, for the response specificity assay to be conducted in breast cancer patients as described below.

Profile PBMCs from Breast Cancer Patients Vs Healthy Donors

Proper transcription of inflammatory response genes may be altered in circulating monocytes already exposed to the tumor microenvironment and may be prognostic for the clinical course of breast cancer patients. Previous studies have shown, for example, that IFNγ signaling is altered in monocytes from ER+ breast cancer patients. Conversely, detection of the type of misregulation of gene programs in monocytes of breast cancer patients would help inform therapeutic options. It is hypothesized that breast cancer patient monocytes would exhibit loss of transcriptomic response specificity due to dysregulation of the gene regulatory strategy of a subset of genes, which can be quantified by information theoretic analyses of stimulus-response scRNAseq data.

Flow cytometry workflow and scRNAseq workflow have been described above. To enable the eventual definition of healthy vs. unhealthy ranges of monocyte response scores for a given set of stimuli, one can apply mutual information calculations and machine learning classifiers to healthy vs cancer patients to identify most informative set of analytes and their interactions. One could compare analyte combinations from both flow and scRNAseq data to determine which might be more informative in reflecting innate immune function in breast cancer patients.

Completion of these studies would help determine which genes, single or combinations, show differential responses, or reduced stimulus-specificity. These genes can be used to establish ranges for response specificity score for healthy innate immune cells vs those from breast cancer patients. As an example from a previous study, this concept and experimental/analysis approaches were applied to innate immune cells from healthy vs Sjogren's Syndrome mice. Stimulus-specific gene sets were identified in that autoimmune disease and both machine learning classifiers and information capacity calculations were able to score the reduced functionality of innate immune cells from the disease model. Different sets of genes would be identified for scoring healthy vs breast cancer monocytes and may also be mechanistically instructive.

Results from these studies would validate new immune biomarkers related to the innate immune cells. It would build a comprehensive network of multiple signaling pathways extended to innate immune cell populations over time in the healthy and cancer immune system. By integrating concepts of information theory (mutual information and channel capacity) it would lead to a novel computational framework that allows us to gain valuable mechanistic insights into the information flow through the immune signaling networks. By the observation of dynamic responses to stimuli, one would be able to create functional scores of immune cell states with clinical implications.

### Example 13

### Evaluation of Monocyte Response Specificity to Assess Innate Immune Health

Innate immune health is a critical determinant of susceptibility to infectious and inflammatory diseases. For instance, monocytes from severely ill COVID-19 patients exhibit defective IFN responses. However, there are currently no clinical measures to assess innate immune health and predict an individual's risk of poor outcomes in diseases such as COVID-19 and cancer.

This example examines how the specificity of monocyte responses is impacted by obesity, which is a major risk factor for severe COVID-19 and other infectious diseases, with the ultimate goal of developing a diagnostic test to predict broad susceptibility to infectious and inflammatory diseases. It is hypothesized that monocytes from obese individuals exhibit inappropriate, less-specific responses. One could identify key indicator genes that can be developed into a clinical diagnostic test to define innate immune health and predict risk for poor outcomes in COVID-19 and other infectious and inflammatory diseases.

In one embodiment, monocytes isolated from PBMCs obtained from lean (e.g. BMI 18.5-25) and obese (e.g. BMI >40) individuals could be stimulated with microbial and inflammatory ligands (e.g. LPS, Pam3CSK4, CpG, poly(I:C), flagellin, R848, TNF-α, IFN-b) for 8 hours, and expression of 500 genes (including cytokines, chemokines, other inflammatory mediators) selected for being highly stimulus-specific in healthy individuals can be examined as described above. The Rhapsody scRNAseq workflow and data analysis described above are used in the experiments. The primary metrics could be classification F1 score and channel capacity. Based on mouse studies, and assuming a common standard deviation as high as +/−20%, with n=10 samples, one can achieve 92% power using a two-sided two-sample t-test, at 0.05 significance level. Clinical records of relevant comorbidities would allow us to stratify the donor population further to identify relevant biomarkers.

### Example 14

### Quantifying Stimulus-Response Specificity Reveals the Functional Health of Macrophages

### Materials and Methods

### Macrophage Cell Culture

Macrophages were obtained by differentiating immortalized myeloid progenitors (iMPs) in DMEM/10% FBS+30% L929 supernatant for a total of 10 days: iMPs were initially thawed into 10 cm non-adherent petri dishes for 3 days. On day 0 of differentiation, cells were then washed once and transferred into differentiation media (DMEM/10% FBS, 30% L929 supernatant, 1% PS, 1% L-Glut, b-Me (1:1000)). Cells were replated into 6 cm plates with new media on day 7, at a density of ˜20k cells/cm². On day 10, the iMP-derived macrophages (iMPDMs) were stimulated with 100 ng/mL lipopolysaccharide (LPS, Sigma Aldrich), 10 ng/mL murine TNF, and 50 μg/mL low molecular weight polyinosine-polycytidylic acid (Poly(I:C)), 100 nM synthetic CpG ODN 1668 (CpG), 500 U/ml IFNβ, or media only Untreated control. For polarized macrophages, cells were incubated in 50 ng/ml IFNγ or 50 ng/ml IL4 for 24 hours prior to stimulation on day 10.

### Peritoneal Macrophage Experiments

C57Bl/6 mice were obtained from Jackson labs. Two male mice were combined for each condition in order to obtain sufficient numbers of cells for the assay: 90 wks old (000664 C57BL/6J), 17 wks old (380050 C57BL/6J/DIO high fat diet (60% fat diet)), 17 wks old (380056 C57BL/6J/DIO controls (10% fat diet)). Peritoneal macrophages were extracted by injecting 10 mL PBS+1% FBS into the peritoneal space, shaking gently, and then pulling out as much fluid as possible, typically ˜8 ml. Macrophages were plated in DMEM +10% FBS and allowed to rest 24 hrs. Floating cells after that time were washed away, and remaining adherent macrophages were stimulated with the same ligand concentrations as for iMPDMs: 100 ng/mL lipopolysaccharide (LPS, Sigma Aldrich), 10 ng/mL murine TNF (R&D), 50 μg/mL low molecular weight polyinosine-polycytidylic acid (Poly(I:C)), 500 U/ml IFNβ, or media only Untreated control. Cells were washed once with cold PBS after 3 hrs of stimulation and lifted into suspension for the Rhapsody scRNAseq assay. The investigators were not blinded to the identity of the animal models during the experiments or outcome assessment.

### Gene Panel Selection Algorithm

To select genes for single-cell targeted gene profiling, existing bulk transcriptomic profiling of macrophage responses were analyzed. Bulk RNAseq data from Cheng et al. (Cell Systems, 4:330-343, 2017) was obtained from GEO GSE68318. Counts were converted to counts per million (cpm) using the package edgeR (Bioinformatics, 26:139-140, 2010), and genes with cpm >4 in at least three samples were retained. Induced genes were gathered by calculating fold changes at each of the 14 stimulus conditions available in the dataset, at each timepoint, against the unstimulated controls. Genes were retained as induced genes if they met the threshold of log 2 (fold change)>2 and p-value <10-5, which resulted in 1502 genes.

Because PCA identifies a new basis that maximizes variance within the rotated data, it was ideal for identifying genes that varied in expression level across different stimuli. PCA was performed centered and unscaled on the induced genes across all time points for the 14 stimuli in the dataset. The loadings matrix obtained from the PCA was used to calculate a rank score for each gene. The rank was computed as the radial distance of each gene j from the origin, over the top 20 PCs: scoreⱼ=Σₓ₌₁²⁰(PCxⱼ)², where PCxⱼ is the component x loadings value for gene j. The top 480 ranked genes were included in the panel, and the remaining 20 genes were manually selected to add genes such as cell type markers, macrophage polarization markers, and transcription factors (Listing 1). As a visual confirmation of the approach, k-means clustering was performed on all induced genes and loadings were colored by cluster. As expected, genes with the highest absolute loadings values in each principal component tended to be spread across different clusters, and the top genes in each principal component exhibited distinct patterns across stimuli.

### Selection of Stimuli for Response Specificity Assay

To identify an optimal set of the most distinct stimuli from the set of 14 used in the bulk transcriptomic data, tensor components analysis (TCA) was performed. TCA is a higher dimensional parallel of PCA— whereas PCA is performed on a genes×sample matrix, TCA is performed on a higher-order tensor by folding the gene expression matrix into a genes×stimuli×timepoint tensor. The bulk RNAseq data consisted of N genes over S stimuli with T timepoints per stimulus, which formed a third-order tensor X of dimensions N×S×T (a three-dimensional array). Tucker decomposition (Psychometrika, 31:279-311, 1966) was performed using the package rTensor. This decomposes the tensor into a core tensor G of dimensions R₁×R₂×R₃, multiplied by a matrix U⁽ⁱ⁾ along each mode,

X=G×₁U⁽¹⁾×₂U⁽²⁾×₃U⁽³⁾

The first five components, which explained 92% of the variance in the data, were retained. The stimulus loadings matrix U⁽²⁾ of dimensions S×R₂ was hierarchically clustered on the first five components, and stimuli that each occupied separate branches of the hierarchical tree were selected. Stimuli that represented whole bacterial organisms or viruses were not selected, in favor of isolated bacterial or viral components.

Rhapsody scRNAseq

To collect the adherent macrophages for scRNAseq using the Rhapsody platform, macrophage cells were washed 1× with cold PBS, then lifted into suspension by incubating at 37 C for 5 minutes with Accutase, which resulted in cell viability typically >85%. Cells were centrifuged at 4 C, 400 g for 5 minutes, and resuspended in PBS+2% FBS. Cells were hash-tagged with anti-CD45-hashtags (BD Rhapsody #633793) and loaded onto the cartridge following manufacturer's instructions (BD Rhapsody #633771), with the following modifications, which helped ensure sufficient cell viability for the subsequent steps: Incubation with hashtags was performed for 30 mins on ice, instead of 20 mins at room temperature; only two washes were performed after hashtag incubation to minimize cell loss. Each cartridge was then loaded with a total of ˜36k cells across 12 hash-tagged samples (˜3k cells/sample). Libraries were prepared according to manufacturer's instructions (BD Rhapsody #633771) and sequenced 2×100 on Novaseq 6000.

### Motif Enrichment

Motif enrichment of induced genes and selected genes was performed using HOMER (Molecular Cell, 38:576-589, 2010), with a motif search range of −1000 to +100 of the TSS of each gene. Individual motif hits were placed into five categories: bZIP (AP1 family TF motifs), IRF (IRF and ISRE motifs), RHD (Rel Homology Domain NFκB family motifs), ETS (Erythroblast Transformation Specific family TF motifs), and Zf (Zinc finger motifs). To summarize the overall enrichment of particular transcription factor families within the gene sets, the average −ln(p value) of motifs in each category was calculated, and a second log transform was taken for plot visualization.

### Gene Ontology

Gene ontology on selected genes versus unselected genes was performed using clusterProfiler (OMICS: A Journal of Integrative Biology, 16:284-287, 2012) against a background of all genes. Cutoff values of p-value <0.01, Benjamini-Hochberg q-value <0.05, and minimum gene set size >5 were used. Ontologies were grouped if they had a similarity proportion greater than 0.7. The top three Biological Processes ontology terms for each group were plotted.

scRNAseq Data Processing

Raw fastq files were processed using the BD Rhapsody™ Targeted Analysis Pipeline (version v1.0) hosted on Seven Bridges Genomics. Distribution-Based Error Correction (DBEC)-adjusted UMI counts (molecules per cell) were used in the downstream analysis. Multiplets, cells with undetermined barcodes, and cells with less than 80 features were removed from the analysis. Due to the selected 500 gene panel comprised of largely inducible genes, the assumption that the total number of RNAs per cell is constant does not hold. Counts were therefore normalized using the package ISnorm, rather than the more standard approach of dividing by total counts per cell. PCA was performed centered and unscaled using the R function prcomp, and UMAP and tSNE were performed on the top 20 PCs.

### Machine Learning Models and Feature Importance

Machine learning classification models were trained using scRNAseq data from naïve macrophages. The data was split 70%/30% into a training group and a testing group. Using only the training data, a random forest model was trained using repeated 10-fold cross validation, with 3 repeats. The parameter mtry, which is the number of variables randomly selected as candidate features for each decision tree split, was set to √(total number of features). As alternative models, weighted k-nearest neighbors and neural network model were also trained on the same dataset. Random forest, weighted kNN, and neural network models were implemented using the R package caret (classification and regression training) (Journal of Statistical Software, 28:1-26, 2008). After the model was trained, the remaining held-out data was tested, with each cell assigned a soft probability prediction for each ligand. The highest probability ligand was the final prediction. Ensemble modeling was performed by majority voting, taking the predicted stimuli from each of the individual models and choosing the most common stimulus predicted for each cell among the different machine learning models. Macrophages of other polarization conditions, M1 (IFNγ) and M2 (IL4), were tested using the model trained on M0 naïve macrophages.

Feature importance was extracted from the trained random forest model, which is calculated by how much information is lost at each node/split of the decision trees. This value was calculated based on Gini impurity: Σᵢ₌₁^{C}(fregᵢ×(1−freqᵢ)), across all unique category labels C. The feature importance is then the product (decrease in node impurity)*(probability of reaching that node), scaled so the top feature has a value of 100.

### Mutual Information Analyses

An information theoretic approach was used to identify either individual genes or combinations of genes providing the highest maximum mutual information between ligand identity and gene expression. Error bars on mutual information calculations were done using 10 bootstraps on 50% of the data. Estimation of maximum mutual information was implemented using the R package SLEMI (PLoS Comput Biol., 15, e1007132, 2019), which uses a statistical learning-based approach to more accurately and more efficiently calculate maximum mutual information for data types with higher dimensional outputs. The max MI was first calculated for each gene individually, using all stimuli (highest theoretical max MI=2.58 bits), as well as for all pairs of stimuli (highest theoretical max MI=1.0 bits). To relate the max MI to the either the mean and variance of each gene, max MI was plotted against either the average Fano factor

$$( avg . FF = ( {\sum }_{i = 1}^{S} ( \frac{{σ}_{t}}{{μ}_{i}} ) ) / S )$$

across all stimuli S for each gene, or the mean squared deviation

$$( M S D = \frac{{\sum }_{i = 1}^{S} {( {x}_{i} - \overline{x} )}^{2}}{S} ) .$$

To estimate the maximum mutual information of the best combination of 1, 2, 3, . . . , N genes, we first started from a list of the top 20 genes that individually had the best max MI value. For each of these single dimension channels, every combination of two genes was scanned, and again ranked the best combination of two genes and retained the top 20. This process was repeated for each additional gene until the gain in max MI for each additional gene leveled off. Retaining only the top 20 sets at each dimension made the calculation more computationally feasible, while still allowing the possibility for gene combinations that are not simply additive of the previous dimension's highest max MI combination.

For gene-specific pairwise calculation of MI, max MI between pairs of stimuli was calculated for each gene at 3 hrs using the single-cell RNAseq data. Max MI was plotted against gene regulatory groups between the two stimuli. Correlation coefficients were calculated using the max MI values for signaling pairs for every NFκB signaling codon, and the max MI values for the corresponding gene expression pairs for every NFκB target gene. Genes without any correlation p-value <0.25 across the six codons were removed from the display. The Pearson's coefficient was plotted on the heatmap, and genes were hierarchically clustered using complete linkage.

### Response Specificity Profile

The Response Specificity Profile is a collection of values that collectively summarize the distinguishability of macrophage responses to different stimuli. The response landscape on which the Response Specificity Profile is calculated was obtained first by principal component analysis performed centered and unscaled on all stimuli across M0, M1 (IFNγ), and M2 (IL4) cells. This initial matrix can be written as =N×P, where N is the total number of measured genes and P is the stimulated single cells of different macrophage subtypes. This matrix rotation of M by PCA represents the landscape of physiological macrophage responses. Calculation of maximum mutual information using the PC scores was then performed for all possible pairs of stimuli. The advantage of using PC scores to perform mutual information analyses lies in the reduction of noise that would otherwise result in overfitting. Overfitting due to the large number of features was observed to result in saturation of the maximum mutual information values to the theoretical maximum for all pairs. Max MI was therefore calculated on the top three PCs (capturing 42% of the variance in the data), using a truncation based on the PCA scree plot, as each subsequent component after Component 5 added only ˜1% more to the variance explained. All error bars were generated by 50 iterations of 50% bootstrap resampling of the complete single cell dataset.

The Response Specificity Profile of new samples was evaluated by projection of the new gene expression data onto the dimensionality-reduced space. The initial PCA was defined as =W^{T}×M, where S is the r×P scores matrix, and W is the N×r loadings matrix, the scores of the new projected data is then given by S_{new}=W^{T}×M_{new}.

Since cells from disease models are projected into the same basis, scores from any new projected data now sit in the same lower-dimensional space and can be compared to the Response Specificity of samples within initially defined response landscape. For new samples, the maximum mutual information between ligand and transcriptomic output was again calculated for all available pairs of ligands using the PC scores. The summary score delta Response Specificity Index (ΔRSI) was generated by the following: ΔRSI=√Σ(RSIₚ−RSIₚ^{M0})² across all pairs of stimuli p.

### Data and Code Availability

Code for data processing and analysis, and processed single cell data, including raw counts and normalized data, are available on the Github repository.

### Results

### Despite Single Cell Heterogeneity, Macrophages Produce Highly Specific Gene Expression Responses to Diverse Immune Stimuli

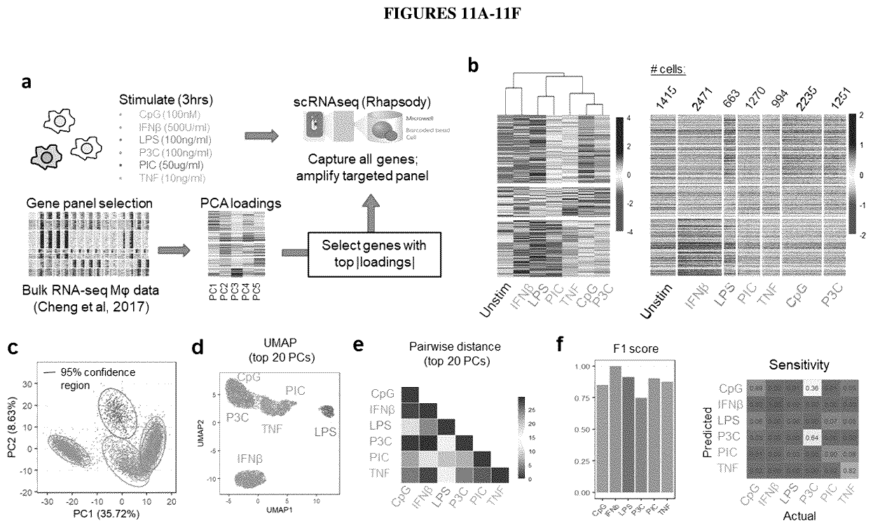

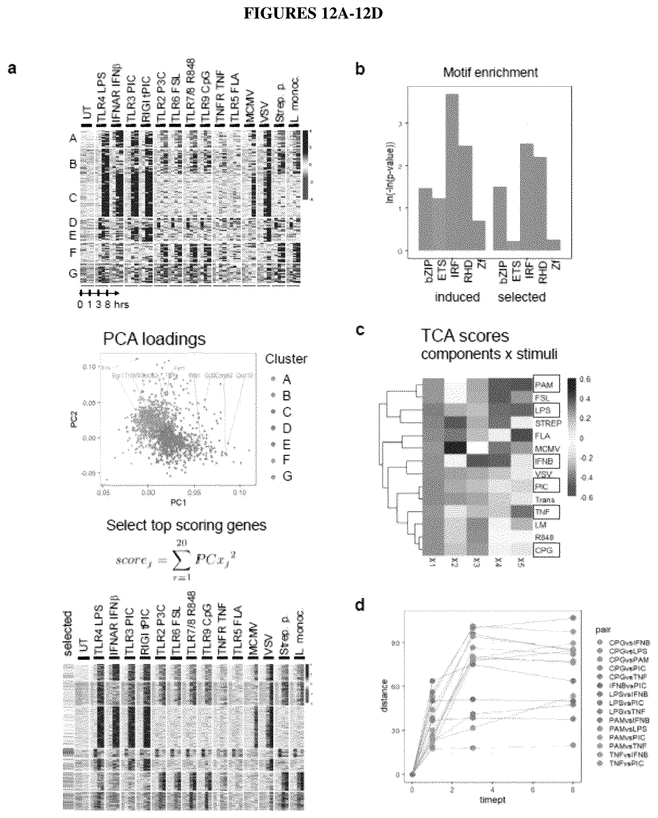

To quantify the degree of Response Specificity in macrophages, an experimental workflow was developed using a targeted mRNA sequencing approach (Adv. Exp. Med. Biol., 1129:63-79, 2019) that was both cost-effective and reduced the technical noise of single-cell genome-wide RNA sequencing approaches (FIG. 11A). A set of 500 macrophage genes was selected via Principal Component Analysis on available macrophages response RNAseq data (Cell Systems, 4:330-343, 2017), using gene loadings to identify the most stimulus-specific genes in an unsupervised manner (Listing 1, FIG. 12A). The selected set of 500 genes showed greater enrichment of NFκB, IRF, and MAPK regulatory features than 1502 stimulus-induced genes, which suggested that these three signaling pathways are the primary drivers of Response Specificity (FIG. 12B).

To identify immune stimuli that would best represent Response Specificity, bulk RNAseq data from macrophages responding to 14 different pathogen or cytokine ligands was analyzed to determine the ligands that induce diverse macrophage responses (FIG. 12A). Tensor components analysis (SIAM Rev., 51:455-500, 2009) across multiple measured timepoints showed that each ligand occupied a non-redundant location, indicating a distinct transcriptomic response at the population level, though some ligands sat closely adjacent in the tensor-decomposed space (FIG. 12C). From the 14 stimuli, 6 were selected that represented a spectrum of gram-positive bacteria (Pam3CSK (P3C)), gram-negative bacteria (LPS), bacterial DNA (CpG), viral nucleic acids (poly(I:C) (PIC)), and host cytokines activating either the interferon (IFNβ) or NFκB (TNF) signaling pathways.

Because gene programs are induced dynamically, the quantification of Response Specificity may depend on the time-point at which gene expression measurements are taken. To compare different timepoints of stimulation, pairwise stimuli distances were calculated at 1, 3, and 8 hrs. Ligand-pair distances were most distinct at 3 hrs (FIG. 12D), reflecting less impact from secondary signaling mechanisms than at the 8 hr timepoint, and thus the 3 hr timepoint was chosen for analysis of Response Specificity. A heatmap comparison of the newly generated single-cell data showed good concordance with the published bulk RNAseq data, while revealing substantial cell-to-cell heterogeneity in expression (FIG. 11B).

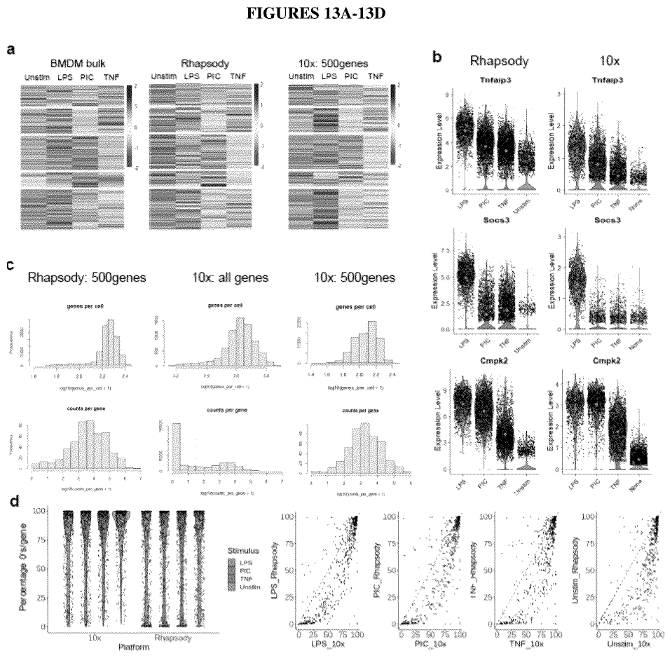

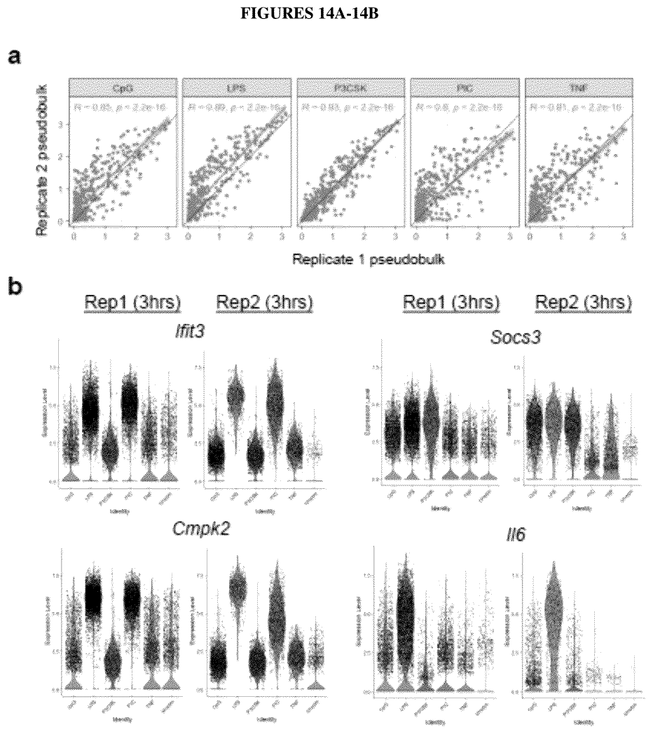

A comparison of the single-cell RNAseq data from the targeted approach (Rhapsody) to the genome-wide approach (10× Genomics) showed that both had similar concordance in their means to bulk data (FIG. 13A) and had comparable distributions to each other (FIG. 13B). Notably, the majority of genes captured only by the genome-wide approach were poorly expressed, further supporting utility of sequencing just the most informative genes through the targeted approach (FIG. 13C). For ˜90% of the genes measured by both approaches, the targeted approach also had a smaller percentage of cells with drop-out data (i.e., genes with zero reads), as each transcript could be sequenced more deeply (FIG. 13D). Taken together, these results suggested that the targeted sequencing approach provided a reproducible (FIGS. 14A-14B), cost-effective means for measuring heterogeneous single-cell macrophage gene expression in response to diverse immune ligands, making it suitable for quantifying Response Specificity.

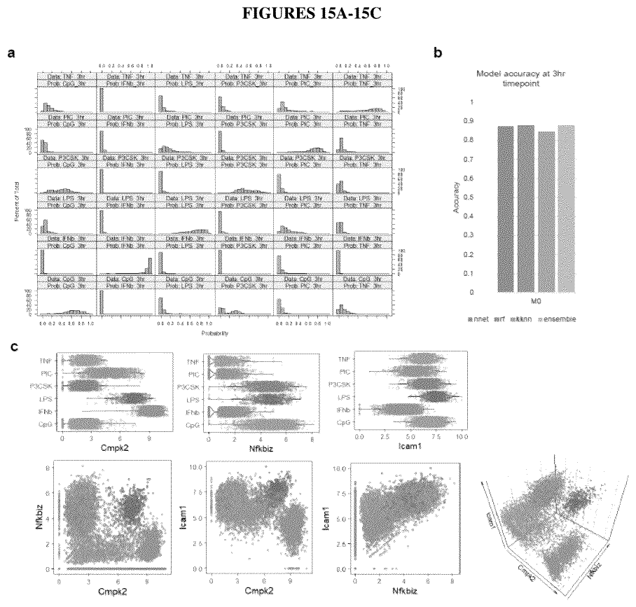

To determine how much single-cell heterogeneity affected the stimulus-specificity of responses, the overlap in gene expression response distributions was assessed next. PCA revealed that IFNβ, LPS, and PIC response distributions were best distinguished, with minimal overlap of their 95% confidence regions on the first two components (44% variance explained), while TNF, CpG, and P3C appeared overlapping (FIG. 11C). UMAP on the top 20 components clarified that TNF could be separated from CpG and P3C on lower components (FIG. 11D), potentially because the latter activate stronger MAPK and non-oscillatory NFκB, while the MyD88-mediated CpG and P3C response distributions remained largely indistinguishable (FIG. 11E). A random forest classifier was trained to determine how well the stimulus could be predicted given a single cell's response transcriptome (FIG. 11F, FIGS. 15A-15B). It was found that IFNβ could be perfectly predicted (F1 score=100%), while the bacterial ligands CpG and P3C were confused with each other and were thus more poorly classified (F1=85% and 74%, respectively). The overall prediction accuracy of 88% across all stimuli indicated a high degree of stimulus-specificity despite cell-to-cell heterogeneity in macrophage responses to ligands (FIG. 11F).

### Despite High Stimulus-Specificity of Gene Expression Programs, Individual Genes can Distinguish Only a Subset of Stimulus Pairs

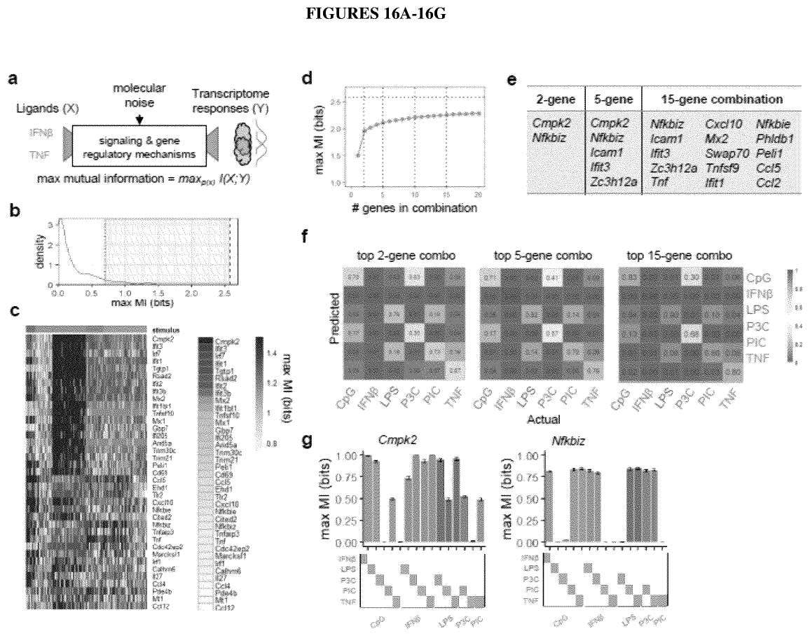

To identify genes that may be driving the observed stimulus-specificity, an information theoretic approach was employed. Ligand information was considered as transmitted through a channel comprised of cell signaling and gene regulatory networks, both affected by pre-existing biological heterogeneity and the stochasticity of biochemical reactions, to produce gene expression responses (FIG. 16A). Under this framework, the maximum mutual information (max MI) describes the certainty about the ligand input given the transcriptome output. One bit equates perfect distinguishability of two ligand response distributions (2¹=2), 2 bits for 4 ligands (2²=4), and 2.58 bits for 6 ligands (2^{2.58}=6). Because mutual information is maximized over all possible input distributions (max MI), this metric provides a comparable absolute quantity for the biological characteristic of Response Specificity that is independent of the underlying data distributions or the mechanisms by which the information is encoded.

It was found that 95% of the measured genes on their own conveyed no more than 1 bit of information (FIG. 16B, Listing 2A and 2B), or ability to distinguish two conditions. Listing 2A shows single genes ranked by their individual contribution to Response Specificity, quantified by maximum mutual information (MI). The top 5 genes found at 3 hours were Cmpk2, Ifit3, Irf7, Ifit1 and Tgtp1. Listing 2B shows single genes ranked by their importance as determined from a random forest machine learning model trained on the single cell macrophage response data. A heatmap of the top-ranking individual genes indicated that rather than each gene having several levels of expression that would separate ligands, most genes displayed an on-or-off expression pattern across single cells, which thus distinguishes only two groups of ligands (FIG. 16C). The single gene most informative for distinguishing stimuli was Cmpk2, a mitochondria-associated gene with reported antiviral and homeostatic functions, which allowed for a maximum mutual information of 1.5 bits (FIG. 16C, Listing 2A and 2B), corresponding to expression distributions sufficiently narrow to define approximately 3 kinds of stimulus-specific responses. Alternate but overlapping means for determining individual gene contribution to Response Specificity are shown in Listings 2A and 2B.

How then can macrophages achieve high Response Specificity? One possibility is that a combination of genes that show low cell-to-cell heterogeneity may specify stimulus-specific responses to diverse stimuli. To assess this possibility, the same information theoretic framework was employed to calculate the maximum mutual information provided by the best-performing combinations of genes. Indeed, the best combination of two genes (Cmpk2 and Wiz) already allowed for a maximum MI of almost 2 bits, with the gain in max MI plateauing to −2.25 bits as larger combinations were tested (FIG. 16D). Interestingly, the majority of genes within the top gene combinations were intracellular proteins controlling nucleotide metabolism (Cmpk2), anti-viral activity and cell death (Ifit3, Ifit1, Mx2), ubiquitination (Peli1), mRNA half-life of inflammatory genes (Zah12a), or phagocytosis (Swap70) (FIG. 16E). Cytokine genes commonly measured by antibody-based assays (Tnf, Cxcl10, Ccl5, Ccl2) had secondary roles (Listing 3). Listing 3 shows combinations of genes, for every dimension of combinations, that confer the highest Response Specificity. Identification of particular combinations of genes that may rank more poorly individually, but together provide more specificity than a combination of the top-ranked individual genes. Machine learning classification using different gene combination sizes confirmed these conclusions. Even just the top two genes performed reasonably well at 70% accuracy, the top 5 genes further improved classification accuracy to 78%, and the top 15 genes performed almost as well as all genes at 85% accuracy (vs. 88% for all) (FIG. 16F, cf. FIG. 11F).

Gene combinations that worked well together did so by distinguishing complementary ligand pairs (FIG. 16G). For example, Nfkbiz alone contributed 0.75 bits to distinguishing CpG and PIC, complementing the inability of Cmpk2 expression to distinguish these ligands (0 bits).

### The Response Specificity of Cytokine Genes is Limited by High Cell-to-Cell Heterogeneity, Despite Having Distinct Expression Means

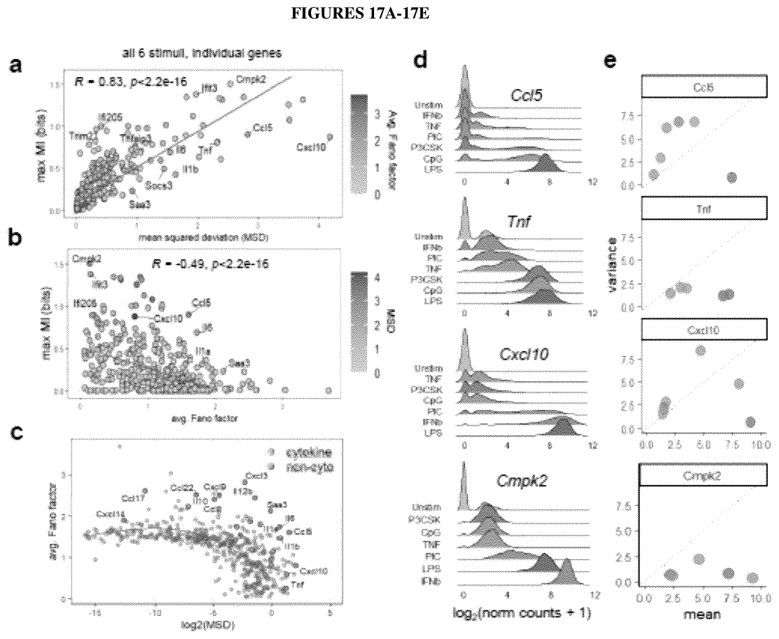

High Response Specificity requires not only that mean population gene expression is distinct, but also that the cell-to-cell heterogeneity is low such that distributions of single cell responses have limited overlap. Hence, whether Response Specificity was limited by small differences in means or wide distributions was investigated. It was found that mean squared deviation (MSD), which summarizes differences in means across ligands, correlated more strongly to Response Specificity (r=0.8) than average Fano factor, which summarizes average gene heterogeneity (r=−0.5) (FIG. 17A). However, a few outliers were evident: some genes with high mean difference (e.g., Ccl5 or Cxcl10) showed unimpressive Response Specificity, with similar max MI to genes with low mean difference (e.g., Ifi205) (FIG. 17A). Plotting the average Fano Factor revealed that these outliers differed in their dispersion: Cxcl10 and Ccl5 displayed much higher heterogeneity (i.e., high avg. Fano factors) (FIG. 17B). In contrast, metabolic and non-secreted genes such as Cmpk2, Ifi205, and Ifit3 had tight distributions (i.e., unusually low avg. Fano factors), and thus high Response Specificity.

Interestingly, a pattern emerged where the Fano Factors of cytokine/chemokine genes were higher than expected, diminishing their Response Specificity despite distinct mean expression levels (FIG. 17C). For example, directly plotting the response distributions of cytokines Ccl5, Tnf, Cxcl10, as well as the non-cytokine gene Cmpk2, showed that Ccl5 was induced with bimodal expression in response to several stimuli, while Cxcl10 and Tnf distributions were very broad (FIG. 17D). These resulted in higher variance/mean ratios than the non-cytokine gene Cmpk2 (FIG. 17E). These results suggested that single cell cytokine/chemokine expression may have limited predictive value in specifying distinct ligand-responses due to single cell heterogeneity.

### The Stimulus Specificities of Immune Response Genes are Due to Selective Deployment of IRF and p38 Pathways, and NFκB Dynamical Features

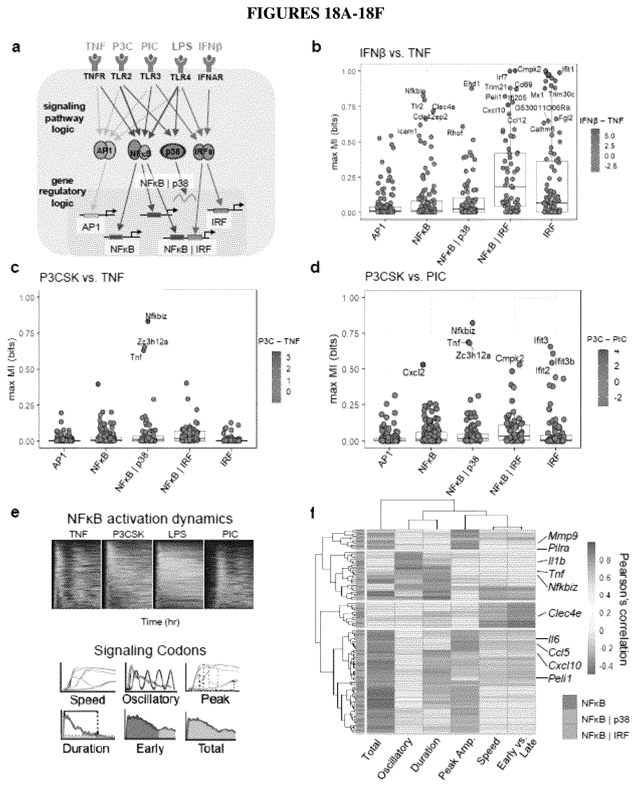

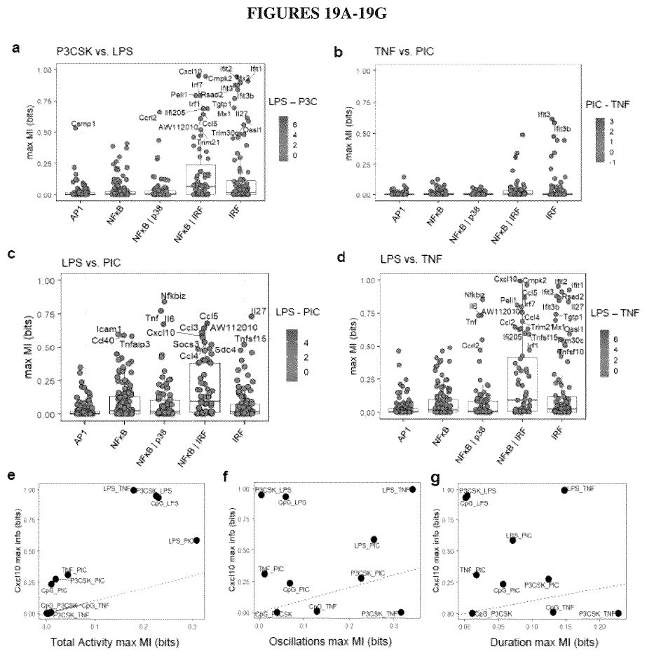

In macrophage responses, four signaling pathways and their downstream gene regulatory factors are combinatorially activated and are responsible for transmitting information about extracellular ligands to the nucleus (FIG. 18A). Their target genes have been categorized into five gene regulatory strategies, namely AP1, NFκB, IRF, NFκBlp38, and NFκBlIRF. The question is which gene regulatory strategies may mediate the high Response Specificity of particular genes, as measured by max MI. Each gene was assigned to a regulatory logic through a curation of prior literature (Listing 4) and the max MI was calculated of every gene for ligand pairs (FIGS. 18B-18D, FIGS. 19A-19D). Listing 4 shows assignment of individual genes to their gene regulatory strategy (GRS) to determine the signaling pathways and genes within each signaling pathway that allow for Response Specificity between pairs or groups of stimuli. For example, contrasting TNF vs. IFNβ, genes within every regulatory logic group showed high specificity (FIG. 18B), a reflection of the fact that each group is activated by only one of the ligands: AP1 and NFκB and NFkBlp38 are activated by TNF but not IFNβ, while IRF genes are activated by IFNβ but not TNF.

In contrast, for the P3CSK vs. TNF stimulus pair, only NFκBlp38 target genes showed specificity because both stimuli activate AP1 and NFκB and fail to activate IRF, while differing only in p38 activation (FIG. 18C). Similarly, for P3CSK vs. PIC, NFκBlp38 targets showed specificity because PIC does not activate p38. However, for P3CSK vs. PIC unlike P3CSK vs. TNF, IRF target genes also contributed to the Response Specificity since PIC activates IRF but P3CSK does not (FIG. 18D). Of note, the max MI of IRF target genes was on average lower for P3CSK vs. PIC than for TNF vs IFNβ, potentially due to highly heterogeneous activation of IRF by the TRIF signaling pathway.

In addition to combinatorial pathway control, the dynamics of NFκB activation also specify gene expression. Stimulus-specific NFκB temporal dynamics involve six NFκB signaling codons that convey information about the stimulus to target genes: “Speed”, “Peak Amplitude”, “Oscillations”, “Duration”, “Total Activity”, and “Early vs. Late” activity (FIG. 18E). To determine which signaling codons may be associated with the stimulus-specificity of NFκB target genes, pairwise specificities of NFκB signaling codons were correlated with the pairwise specificities of NFκB target genes (FIGS. 19E-19G). It was found that NFκB target genes differed in which signaling codons they were associated with (FIG. 18F). These distinctions may be reflective of genes employing distinct gene regulatory mechanisms such as an incoherent feedforward loop that decodes “Peak Amplitude”, long mRNA half-life or slow chromatin opening steps that decode “Duration”, or the requirement for de novo enhancers that distinguishes “Oscillations”.

### Cytokine Polarization Causes Complex Modulation of the Response Specificity of Specific Genes to Specific Stimuli

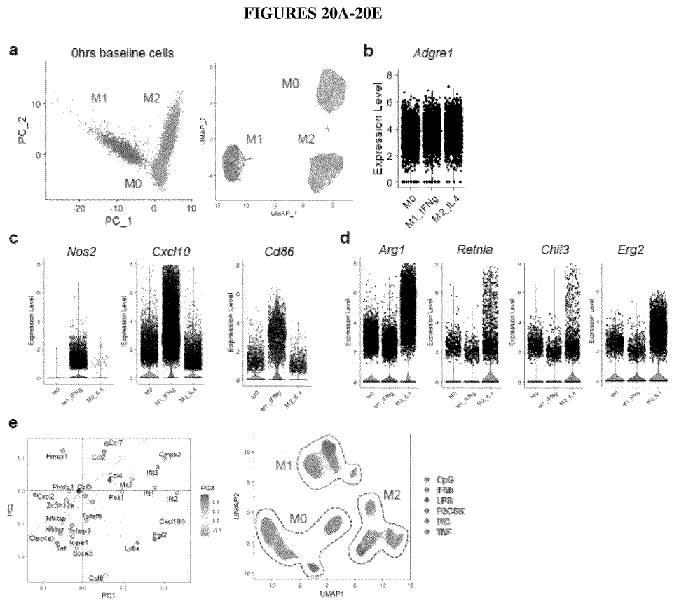

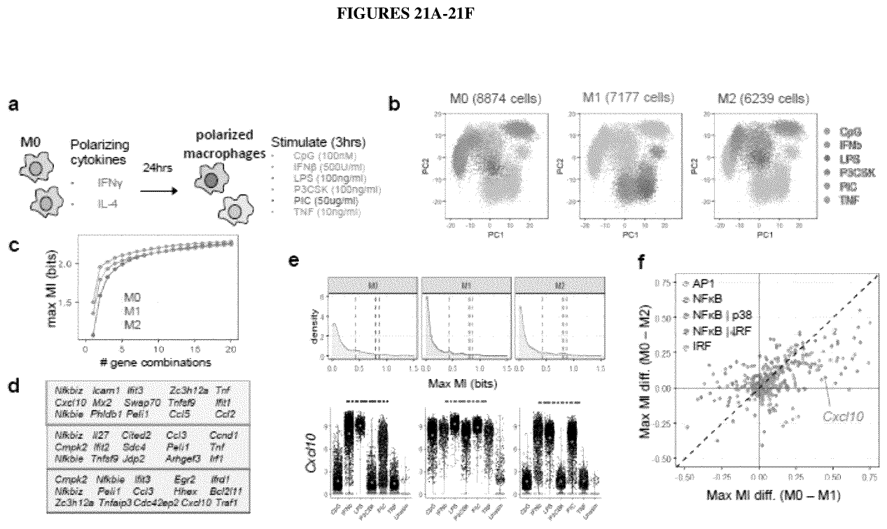

Macrophages show remarkable functional pleiotropy that is dependent on microenvironmental context. Thus, polarization by prior cytokine exposure may alter their capacity for stimulus-specific responses. To test this hypothesis, macrophages were polarized into M1 (IFNγ) and M2 (IL4) states that represent opposing ends of the macrophage polarization spectrum (FIG. 20A), and single-cell stimulus response data was generated for the six ligands (FIG. 21A). Polarized M1 (IFNγ) and M2 (IL4) macrophages expressed macrophage marker Adgre1 (FIG. 20B) and the appropriate polarization markers (FIGS. 20C-20D). PCA and UMAP projections of response distributions revealed that stimulus responses for polarized macrophages were distinct (FIG. 21B, FIG. 20E), but M1 (IFNγ) response distributions in particular appeared more overlapping than those for M0 naïve macrophages.

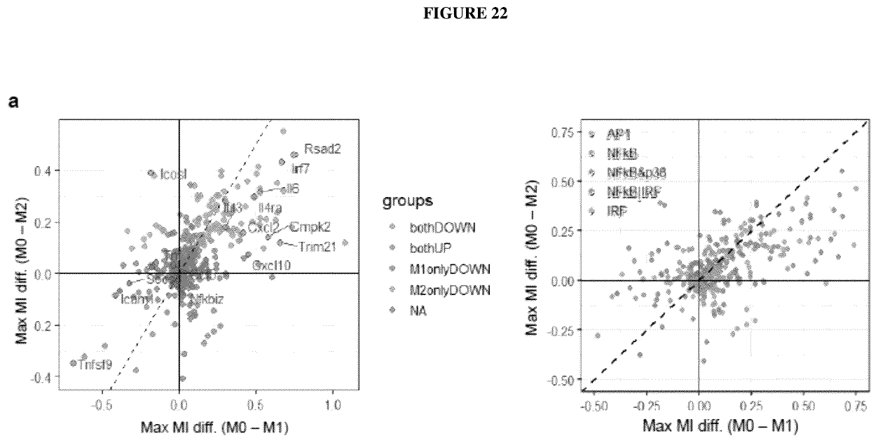

It was found that Response Specificity was reduced in both polarization states for every set of the best gene combinations, as calculated by max MI, indicating that polarized macrophages may function more as specialized effectors and less as sentinels that serve a primary role of distinguishing immune threats (FIG. 21C, Listing 3). Interestingly, it was further observed that the genes within each of the best gene sets were different for each polarization state (FIG. 21D). This was corroborated by examining one gene, Cxcl10, which was included in the best gene combinations in M0 and M2 (IL4) conditions but not in M1 (IFNγ). Indeed, in M1 (IFNγ) macrophages, Cxcl10 was promiscuously rather than stimulus-specifically activated, and no longer carried stimulus-specific information about any pairs of stimuli (FIG. 21E). This change in Response Specificity could be ascribed to the IRF pathway; several other NFκBlIRF target genes (Cxcl10, Cmpk2, Ifit3, and Trim21) lost specificity in M1 (IFNγ) macrophages (FIG. 21F, FIG. 22), reflecting the fact that in the presence of IFNγ conditioning such genes only require some NFκB activity to be induced.

While a global analysis indicated that polarization diminished macrophage Response Specificity, the genes most affected differed for each macrophage state. In addition, a smaller set of largely NFκB response genes also showed increased rather than diminished Response Specificities under each polarization condition (FIG. 21F). Such nuanced findings preclude the use of a single gene or even a single set of genes to quantify Response Specificity over multiple macrophage states. The Response Specificity of macrophages thus may not be appropriately characterized by a single number, but rather a higher dimensional profile.

The Response Specificity Profile of Stimulus Pairs Assesses the Functional State of Macrophages, and Readily Distinguishes M0 vs. M1 vs. M2 Macrophages

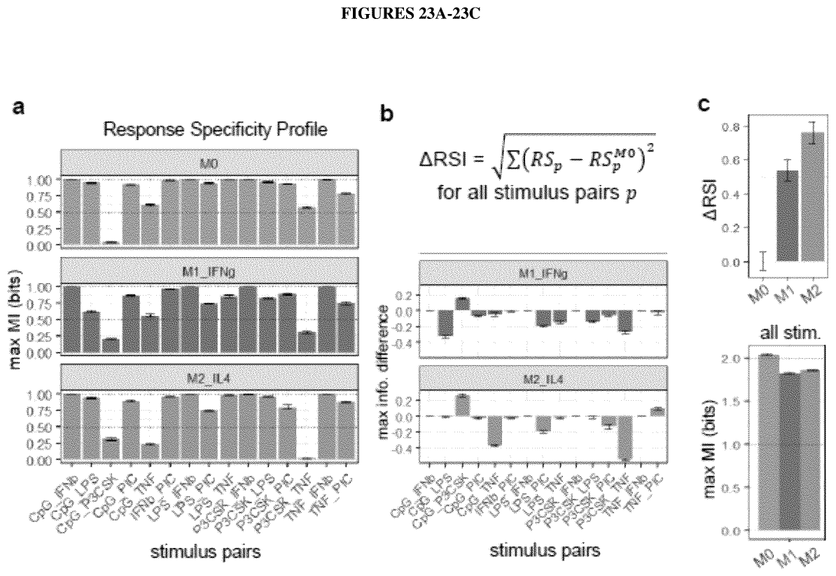

Because the Response Specificity of particular genes was distinctly altered by each cytokine context (FIG. 21D), and genes differed in which stimulus-pair they could distinguish (FIG. 16G), an approach was developed to profile the max MI of all 15 stimulus pairs defined by the 6 stimuli, via PCA projection of single-cell stimulus-response transcriptomes onto the macrophage response landscape defined by all possible stimulus responses (FIG. 23A, Listing 5). Listing 5 shows genes ranked by PCA loadings from PCA on combined M0, M1, and M2 scRNAseq macrophage response data. The PCA forms the macrophage response landscape, and all genes provide a weighted contribution to the Response Specificity Profile. As the Response Specificity Profile was calculated using the first three principal components, the genes with the highest absolute value weights on these components are most important for the Profile and the overall delta Response Specificity Index. The resulting Response Specificity Profile of M0, M1 (IFNγ), and M2 (IL4) macrophages showed specific differences for some of the stimulus pairs tested (FIG. 23A). Particularly, M1 (IFNγ) macrophages were impaired in distinguishing bacterial stimuli (LPS-CpG; LPS-P3C), while M2 (IL4) macrophages were not. Meanwhile, M2 (IL4) macrophages were more impaired in distinguishing host-cytokine vs. bacteria (TNF-CpG, TNF-P3C). In both polarization states, CpG-P3CSK specificity, which had been the least distinguishable stimulus pair for M0 macrophages, was enhanced.

Calculating the difference in max MI from the M0 state, and thereby the Delta Response Specificity Profile, provided a characteristic signature of the functional state of the macrophage (FIG. 23B). This profile could also be summarized succinctly in a single number, the delta Response Specificity Index (ΔRSI), which provided a clearer indication of differences among polarization states than the mutual information calculated on all stimuli together (FIG. 23C). It was found that the overall ΔRSI of M2 (IL4) macrophages was greater than that of M1 (IFNγ) macrophages, as a result of M2 (IL4) macrophages showing a greater loss of specificity in distinguishing host vs. bacterial ligands. Thus, the Response Specificity Profile and ΔRSI revealed a signature of pairwise response specificity scores associated with the function of each macrophage type, whether naïve, M1 (IFNγ) or M2 (IL4).

Peritoneal Macrophages from Old and Obese Mice Show Distinctive Alterations in their Response Specificity Profiles

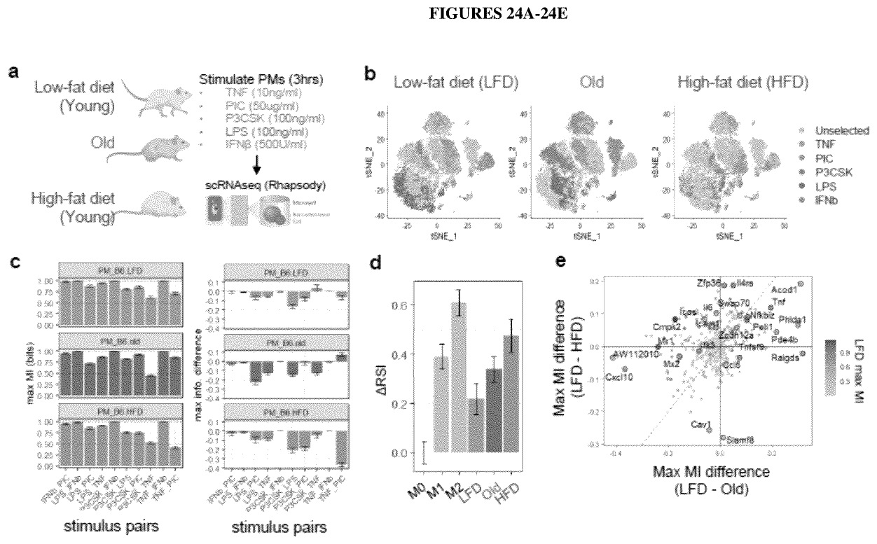

Next, whether the Response Specificity Profile might reveal aberrations in macrophages derived from mice at risk for chronic inflammatory disease was tested. Peritoneal macrophages (PMs) were taken from healthy mice (17 weeks old), old mice (90 weeks old), and high-fat-diet obese mice (17 weeks old) and the Response Specificity workflow was performed using five ligands: LPS, TNF, PIC, P3C, and IFNβ (FIG. 24A). Visualization of the resulting single-cell data suggested that old mice had aberrant IFNβ-response distributions, while obese mice had aberrant PIC-response distributions (FIG. 24B). The Response Specificities for the data were calculated (FIG. 24C) and it was noted that the PMs from young, healthy mice had an overall ΔRSI most similar to naïve M0 macrophages (FIG. 24D). A closer inspection of the Response Specificity Profile showed that PMs from old mice showed the most diminished specificity for IFN-activating stimulus pairs (e.g. LPS-PIC), akin to what we observed with M1 (IFNγ) macrophages. PMs from high-fat diet obese mice had decreased specificity across all stimulus pairs, but particularly in distinguishing cytokine vs. viral (TNF-PIC) responses. The differences observed through calculation of the Response Specificity Profile on stimulus subsets suggest an importance to comparing multiple subsets of stimuli in evaluating innate immune function.

To identify individual genes that showed particularly high losses of Response Specificity in these in-vivo-conditioned macrophages, max MI values for each gene were compared (FIG. 24E). It was found that macrophages from both old and obese mice lost specificity in the cytokine Tnf, but also in metabolic gene Acod1, and upstream TLR signaling network proteins such as Peli1, Nfkbiz, and Phlda1. Normal function of these genes has been implicated in resistance to septic shock, macrophage response to atherosclerosis, and protection from autoimmune disease. Interestingly, a couple of genes showed higher stimulus-specificity in disease conditions. In old mice, genes with the highest gains in specificity were the cytokine Cxcl10 and cytokine regulator Aw112010, which is required for mucosal immunity; and in obese mice, Cav1 and Slamf8, which play roles in macrophage differentiation and migration. This suggested that macrophage responses in disease conditions deviated from healthy through both losses and gains of stimulus-specificity in individual genes: losses indicating responses that are too promiscuous potentially causing conditions for autoimmunity to arise, and gains indicating responses that are too restricted to a particular stimulus, potentially diminishing core functions of macrophages in innate defenses or resolution of tissue inflammation and damage.

Taken together, quantifying the Response Specificity of macrophages revealed that this functional hallmark of immune sentinel cells is affected not only by polarizing cytokines used in pre-conditioning regimes in vitro, but also by the microenvironments in vivo that are evidently distinct in obese and old mice. The observed differences in Response Specificity suggest that quantifying post-stimulation single-cell response distributions could be valuable for assessing the health of macrophage function.

### DISCUSSION

Mounting stimulus-appropriate immune responses is a key property of healthy macrophage function. Macrophage Response Specificity is a function of the stimulus-specific engagement of signaling pathways and may be diminished by molecular noise that results in cell-to-cell heterogeneity. While Response Specificity of one macrophage immune signaling pathway has been characterized, the Response Specificity of immune gene expression responses arising from all macrophage signaling pathways has remained unquantified. By developing the experimental and computational tools to do so, it was found that the high gene expression specificity observed was generated by sufficiently narrow response distributions in combinations of genes that respond with distinct patterns across stimuli, but that the contribution of often-measured cytokine genes was limited by high cell-to-cell heterogeneity of expression. Mechanistically, it was found that Response Specificity is generated by stimulus-specific activation of interferon or MAPKp38 signaling, or by differences in NFκB dynamics Loss of IRF gene specificity by microenvironmental polarization was the key driver in altering Response Specificity Profiles. Given that Response Specificity is context-dependent, peritoneal macrophages from old and obese mice were profiled, revealing highly specific changes in the Response Specificity Profile that reflected a different functional health status. These findings suggest that Response Specificity could be a means to characterize the innate immune health of human donors.

The ability to measure and subsequently quantify Response Specificity was enabled by a quantitative assay for cost-effective, reliable scRNAseq. The targeted sequencing approach pursued here resulted in less technical noise than genome-wide approaches, due to improved reverse transcriptase efficiency and increased sequencing depth per gene. However, even with technical improvements, the remaining measurement noise still may result in underestimates of the true Response Specificity. Future efforts on smaller gene lists may allow the use of even less noisy measurement approaches like single molecule fluorescence in situ hybridization (smFISH) that capitalize on the smaller sets of informative genes identified in this study.

The information theoretic approach used here to quantify Response Specificity, previously employed to quantify the information transmission capacity of signaling pathways, led to the finding that only a few of each pathway's target genes maintained the information available from signaling activity. Theoretically, information loss from signaling to gene expression is minimal without noise, while with noise, maintaining minimal information loss is only possible under select optimal promoter or chromatin conditions. A further consideration is that an individual gene can be regulated either by a single pathway or by multiple pathways, for example the NFκB target gene Tnf whose mRNA half-life is regulated by stimulus-induced MAPKp38. This combinatorial control explains why in principle some single genes like Tnf can hold more information than available from a single signaling pathway. But to achieve high gene expression Response Specificity in either scenario, signaling information must be interpreted by gene regulatory mechanisms without amplifying the cell-to-cell heterogeneity in signaling activity that already degrades stimulus-specificity, and also without introducing further heterogeneity through pre-existing chromatin heterogeneity or molecular noise. In this context, it is not surprising that the Response Specificity of most individual genes was low. However, as macrophages do not rely on only a single gene to mount a biological response to a specific immune threat, even with information loss at each particular promoter, the overall Response Specificity observed through combinations of a few genes from complementary pathways was still high.

Within the body, macrophages are exposed to polarizing microenvironments in physiological scenarios, as well as in pathological inflammatory contexts such as aging or obesity. As circulating cells, they are potentially reporters of even localized infections. Indeed, profiling the transcriptome or epigenome of circulating cells or macrophages has revealed molecular markers or signatures that are prognostic for therapeutic efficacy or alternative disease courses, such as in persistent infectious disease and cardiovascular and autoimmune diseases. However, as seen in human COVID-19 studies, it can be unclear which individuals have poor or vigorous immune health until they are challenged by infection. Here, it is considered that macrophage functions are deployed in response to immune threats and that stimulus responses are a function not only of the steady-state transcriptome or epigenome but also the dynamics of signaling complexes, membranes, and transport rates. It is reasoned that macrophage responses to different stimuli reveal a functional pleiotropy not evident at steady state, and that the stimulus-specific deployment of functions is key to healthy immunity. Indeed, it was found that macrophages conditioned in vitro by defined polarizing cytokines, as well as macrophages isolated from obese or old mice, showed distinctly altered Response Specificity profiles due to both cytokine genes and regulators within macrophage metabolic and signaling pathways.

In developing the Response Specificity Profile, it was found that analyzing single genes and pairs of stimuli provided more insight than aggregating the data together. For example, Response Specificity for each pair of stimuli differed for each polarization condition, but this information is lost when calculating Response Specificity for all stimuli at once. Instead, an aggregate score of alterations in the Response Specificity Profile (delta RSI) provide a first indication of differences, and the full Response Specificity Profile pinpointed aberrancies in select stimulus pairs that may be a diagnostic for a specific condition, such as macrophages from obese mice confusing TNF and PIC responses. For initial surveys of Response Specificity Profiles, measuring the expression of a large number of genes in response to multiple stimuli is important, as the most informative genes and stimulus comparisons are different across various macrophage states. A PCA approach was employed to characterize the response landscape and identify genes important across conditions, by using the gene weights in principal components. But the Response Specificity of individual genes differed greatly between the two disease models tested here, emphasizing the importance of gene-by-gene analysis in characterizing the Response Specificity Profile to make biologically meaningful predictions.

Characterizing the macrophage Response Specificity Profile may prove useful in clinical scenarios. Many steady-state metrics of immune health exist, such as the complete blood count that is a mainstay of clinical lab tests. However, tests for functional immune responses, already used in select clinical scenarios such as tuberculosis testing or allergy testing, are also rapidly emerging. Multiple such assays rely on ex vivo stimulation of extracted clinical samples to diagnose immunosuppression or phagocytic ability, and have included transcriptomic profiling studies of stimulus-responses on peripheral blood that identified inter-individual variation among healthy donors and the genes driving those differences. As seen with existing assays, to what extent stimulus-response measurements provide more reliably prognostic information than steady-state molecular profiling may depend on the health condition being studied.

The assay of single cell macrophage responses presented herein may identify outlier response cells within each donor sample through the Response Specificity Profile, that may be associated with risk for aberrant inflammatory responses or diminished innate immune defenses, or that may be reflective of an ongoing inflammatory or infectious condition that is not otherwise presented. The Response Specificity Profile allows new samples to be compared readily to a healthy range, a property which could be the basis for a clinically deployable measure of innate immune health. The stimulus-response data may also identify specific genes with aberrant response distributions in patient cohorts or in individual donors. Identifying such genes may provide cost-effective prognostic markers for specific cohorts or point to the underlying etiology of poor immune function. To realize this promise, large-scale clinical studies will be required to establish connections between Response Specificity Profiles and risk for disease. However, as a quantifiable property of macrophage function that changes with conditioning cytokines or states of health and disease, macrophage Response Specificity Profile may be a viable approach for measuring the health of innate immune function in the clinic.

### Example 15

### Single-Cell Gene Expression Dynamics Enable Macrophage Response Specificity and Reveal Cell Functional States

Macrophages respond to immune threats via the dynamic induction of hundreds of immune response genes. However, technological limitations have precluded the measurement of temporal trajectories of gene expression at single cell resolution. The present example describes a method, scREAL-TIME, to model the ensemble of single-cell real-time expression trajectories that account for time-series scRNAseq from stimulated macrophages. It was found that dynamical information from single-cell macrophage expression trajectories held a much greater capacity for stimulus-response specificity than apparent from time-point scRNAseq measurements, and uncovered previously unmeasurable correlations in the expression of pro-inflammatory cytokines. The heterogeneity of dynamical features of gene expression was highly dependent on microenvironmental context. Consequently, the polarization state of single cells could be predicted more readily by response dynamics than by steady-state or time-point response measurements. The findings presented herein emphasize the importance of accounting for dynamical information about the cellular transcriptome in characterizing the functionalities and functional states of immune cells.

### Materials and Methods

### Macrophage Cell Culture

Macrophages were generated by differentiating immortalized myeloid progenitors (HoxB4 iMPs) as previously described. HoxB4-iMPs initially thawed and expanded in 10 cm non-adherent petri dishes for 3 days. They were then washed once and placed in differentiation media for a total of 10 days (DMEM/10% FBS, 30% L929 supernatant, 1% PS, 1% L-Glut, b-Me (1:1000)), with replating onto 6 cm plates with new media on day 7, at a density of ˜20k cells/cm2. For polarized macrophages, cells were incubated in 50 ng/ml IFNγ or 50 ng/ml IL4 for 24 hours prior to stimulation on day 10. On day 10, macrophages were stimulated with one of six stimuli: 100 ng/mL lipopolysaccharide (LPS, Sigma Aldrich), 10 ng/mL murine TNF, and 50 μg/mL low molecular weight polyinosine-polycytidylic acid (Poly(I:C)), 100 nM synthetic CpG ODN 1668 (CpG), 500 U/ml IFNβ, or media only Untreated control. Samples were collected at multiple timepoints post-stimulation, including 15 or 30 minutes, 1 hr, 3 hrs, 5 hrs, and 8 hrs.

Macrophage scRNAseq

To collect the adherent macrophages for scRNAseq using the BD Rhapsody platform, macrophage cells were washed 1× with cold PBS, then lifted into suspension by incubating at 37 C for 5 minutes with Accutase, which resulted in cell viability typically >85%. Cells were centrifuged at 4 C, 400 g for 5 minutes, and resuspended in PBS+2% FBS. Cells were hash-tagged with anti-CD45-hashtags (BD Rhapsody #633793) and loaded onto the cartridge following manufacturer's instructions (BD Rhapsody #633771), with the following modifications, which helped ensure sufficient cell viability for the subsequent steps: Incubation with hashtags was performed for 30 mins on ice, instead of 20 mins at room temperature; only two washes were performed after hashtag incubation to minimize cell loss. Each cartridge was then loaded with a total of ˜36k cells across 12 hash-tagged samples (˜3k cells/sample). Libraries were prepared according to manufacturer's instructions (BD Rhapsody #633771) and sequenced 2×100 on Novaseq 6000.

scRNAseq Processing

Raw fastq files were processed using the BD Rhapsody™ Targeted Analysis Pipeline (version v1.0) hosted on Seven Bridges Genomics. Distribution-Based Error Correction (DBEC)-adjusted UMI counts (molecules per cell) were used in the downstream analysis. Multiplets, cells with undetermined barcodes, and cells with less than 80 features were removed from the analysis. Due to the selected 500 gene panel comprised of largely inducible genes, the assumption that the total number of RNAs per cell is constant does not hold. Counts were therefore normalized using the package ISnorm, rather than the more standard approach of dividing by total counts per cell. PCA was performed centered and unscaled using the R function prcomp, and UMAP and tSNE were performed on the top 20 PCs.

scREAL-Time

Dimensionality Reduction: Principal Component Analysis (PCA) was performed on the time-series single-cell RNAseq data. PCA was performed centered and unscaled across all stimuli simultaneously, generating a lower dimensional data embedding that captured the gene expression information of individual cells.

k-means clustering: It is assumed in this method that many representative archetypes of cells can estimate the spectrum of behavior of individual cells. Conceptually then, with enough archetypes linked combinatorially across timepoints, the full range of heterogeneous behavior of individual cells can be estimated in a manner that minimizes the heterogeneity arising from technical noise. k-means clustering was utilized on the top 50 PC scores with k=20 to determine archetypes at each time point. At any given measured time-point, a cell may be classified as a member of one of k archetypes. For each archetype at a given time point, a consensus measurement was utilized—an average value for each PC score determined by the members of said archetype from the data.

Weighted Random Walks: The probability of archetype connections across timepoints was labeled by the density of cells belonging to that archetype and the Euclidean distance of the mean of each archetype. Upon assigning these probabilities, random walks were generated where each simulation belonged to a particular observed archetype at a given timepoint, adopting the archetype's consensus PC measurements.

Spline fitting: For each random walk, unmeasured timepoints were interpolated across by fitting polynomial spline interpolants with three degrees of freedom. This linked the lower dimensional cell-level data, resulting in a trajectory for each cell in PC space, with gene expression trajectories for each cell embedded and recoverable from the original PCA loadings.

Recovering gene trajectories: The approximately invertible property of PCA was utilized to invert dimensional reduction transformation using the top 50 PCs, requiring use of the loadings matrix to scale and rotate the lower dimensional cell-based data. This back projected the random walk trajectories from the lower dimensional space to reflect individual gene trajectories for every cell. Each random walk, i.e., each cell, was subsequently represented as a continuous curve in gene expression space.

### Calculation of Trajectory Features

For comparing stimulus-specificity, trajectories were first scaled 0-1 across all stimuli, and the trajectory features were calculated on the scaled trajectories. This was necessary as spline fitting across principal components generated occasional negative values for some cells, especially at the beginning of the trajectory. Scaling maintained the same relative dynamics and amplitudes, and increased the reliability of the trajectory feature calculation. Integral was calculated by integrating each single cell trajectory curve from 0 to 8 hours. Peak amplitude was calculated by taking the maximum value over 8 hours. Max log fold change was calculated for each gene by taking the fold change between the peak amplitude and the same cell's starting value at 0 hours. Activation speed was calculated by taking the derivative of the curve at 1 hour.

### Calculation of Mutual Information

An information theoretic approach was used to identify either individual genes or combinations of genes providing the highest maximum mutual information between ligand identity and gene expression. Error bars on mutual information calculations were done using 10 bootstraps on 50% of the data. Estimation of maximum mutual information was implemented using the R package SLEMI, which uses a statistical learning-based approach to more accurately and more efficiently calculate maximum mutual information for data types with higher dimensional outputs. The max MI was first calculated for each gene individually, using all stimuli (highest theoretical max MI=2.58 bits), as well as for all pairs of stimuli (highest theoretical max MI=1.0 bits).

### Results

### Time-Course Single-Cell RNAseq Data Reveals Context-Dependent Heterogeneity in Macrophage Responses

In response to inflammatory stimuli, macrophages dynamically transcribe hundreds of immune response genes. Cell-to-cell heterogeneity in the expression of these response genes arises at each regulatory step, including the kinetics of ligand-receptor binding, downstream adapter recruitment, kinase activation, and transcription factor localization, all of which may also be affected by context-dependent signaling crosstalk and chromatin remodeling.

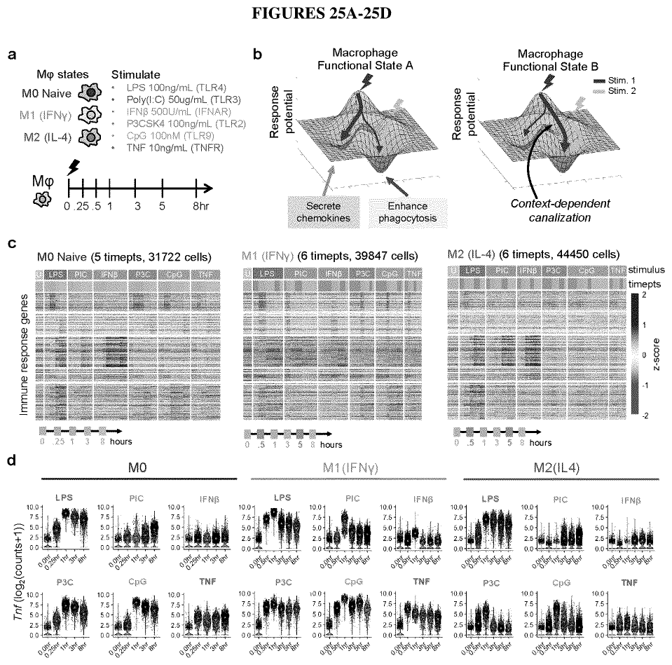

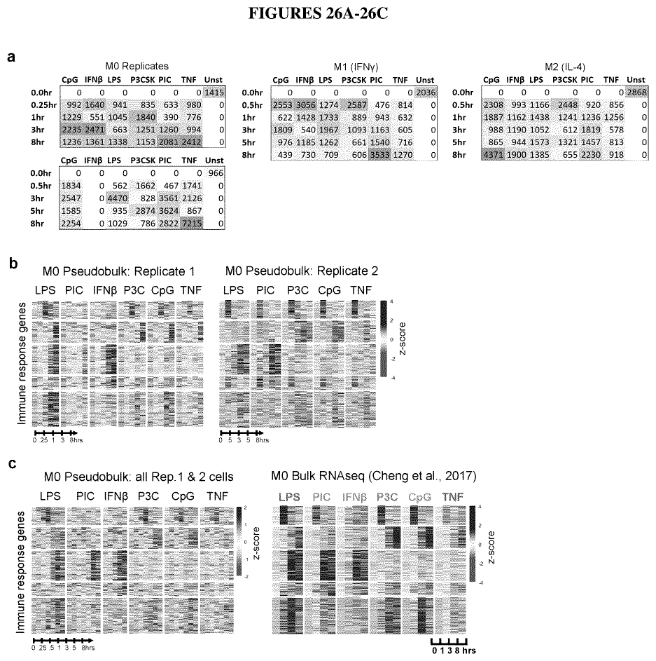

To measure context-, stimulus-, and time-dependent single-cell macrophage responses, the BD Rhapsody platform was used to perform targeted scRNAseq on >100,000 individual macrophages (FIG. 26A) that were stimulated with a panel of six immune ligands (FIG. 25A). The studies focused on 500 induced immune-response genes previously identified as providing high stimulus-response specificity. A set of distinct ligands each binding a distinct pattern recognition receptor or cytokine receptor was used, including pathogen components that bind Toll-Like Receptors (TLRs), LPS (TLR4), poly(I:C) (TLR3), Pam3CSK4 (TLR2), CpG (TLR9), and cytokines TNF (TNFR) and IFNβ (IFNAR) that activate either NFκB or IRF signaling pathways. Cells were collected for scRNAseq data over a fine time-course to capture both the early and late phases of macrophage immune responses (0, 0.5, 1, 3, 5, and 8 hours). Replicate time-series of the M0 naïve macrophages were concordant (FIG. 26B), and pseudobulk patterns of averaged single-cell data were consistent with previously published population-level macrophage stimulus-response RNAseq data (FIG. 26C).

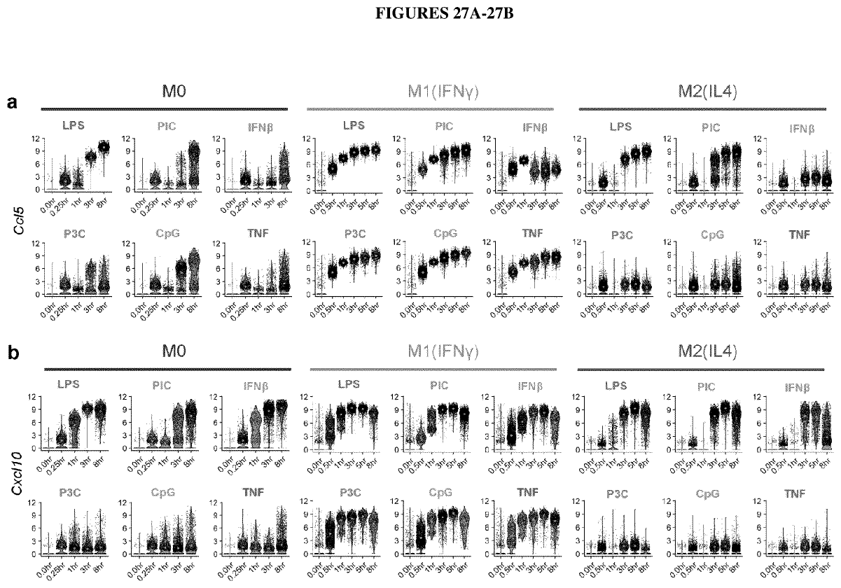

Conceptually, polarizing macrophages with microenvironmental cytokines pushes stimulus-responses into different channels across the response landscape (FIG. 25B). These channeled responses result in macrophages with altered functions, which are not evident without stimulus-perturbations that activate the affected regulatory pathways. To determine the impact of context-dependent signaling and epigenetic states on the timing, amplitude, and heterogeneity of gene expression, macrophages of three different polarization states that canonically represent the proinflammatory or anti-inflammatory states of macrophages: M0, M1 (IFNγ), and M2 (IL4) were stimulated. In total, the resulting single cell stimulus-response data consisted of 500 immune response genes profiled in time series, with each gene consisting of thousands of single-cell data points across stimuli, time points, and polarization states (FIG. 25C), including differing induction dynamics and levels of heterogeneity for genes of related function and regulation (e.g., Ccl5 and Cxcl10) (FIGS. 27A-27B).

Inspecting specific genes revealed timepoint-, stimulus-, and polarization state-dependent changes in expression heterogeneity. For example, in response to LPS, the expression of the proinflammatory cytokine Tnf exhibited high single-cell heterogeneity from to 1 hour in both M0 and M2 (IL4) macrophages, but lower heterogeneity in M1 (IFNγ) macrophages (FIG. 25D). However, the time-series scRNAseq data, being unlinked across time, failed to capture a fundamental aspect of a single-cell's immune response transcriptome: the dynamical trajectory of gene expression. Single-cell gene expression fold changes, peak amplitudes, or accumulated total in mRNA abundance could not be gleaned from the scRNAseq datasets.

Identifying the Ensemble of Single-Cell Expression Trajectories that Account for Time-Series scRNAseq Data

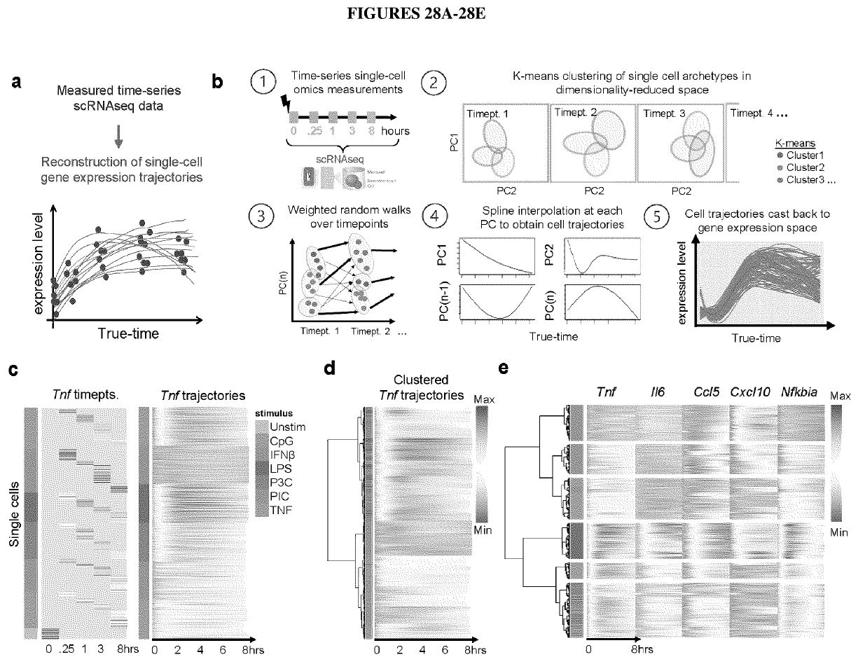

To examine the amount of stimulus information contained in immune response gene trajectories, a method was developed to identify the ensemble of single-cell real-time transcriptomic trajectories that would account for the measured time-series scRNAseq data: single-cell Real-time Ensemble Archetype Linkage of Time-series Measurements (scREAL-TIME) (FIG. 28A). In developing scREAL-TIME, it was aimed to estimate the simplest trajectories that preserved key characteristics of the data, including 1) maintaining measured gene-gene correlations in single cells across time, 2) recapitulating mean and variances of the measured single-cell data, and 3) linking cells within distributions across timepoints in a manner that would reflect a cell's natural continuity of gene expression. To this end, dimensionality reduction (PCA) was first performed on all the timepoints to preserve cellular gene-gene correlations within PCA loadings (FIG. 28B, step 1). k-means clustering was then performed on the top 50 component scores to identify cell archetypes at each timepoint from single-cell distributions (FIG. 28B, step 2), and connected high-probability cell archetype matches based on Euclidean distance across timepoints using weighted random walks (FIG. 28B, step 3). This weighting makes every permutation of links possible but reduces the probability of trajectories across less likely paths. Spline fitting was used to smooth each cell's trajectory across all principal components (FIG. 28B, step 4). Using the original PCA loadings, the cell trajectories were then projected back to the original space to recover correlated transcriptome trajectories for each cell, resulting in expression trajectories of individual genes over real-time (FIG. 28B, step 5).

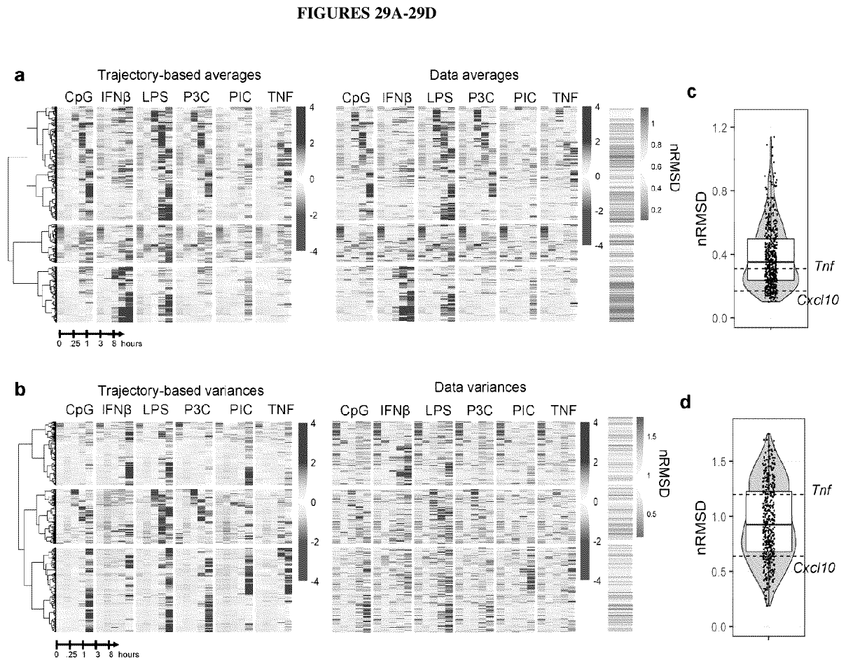

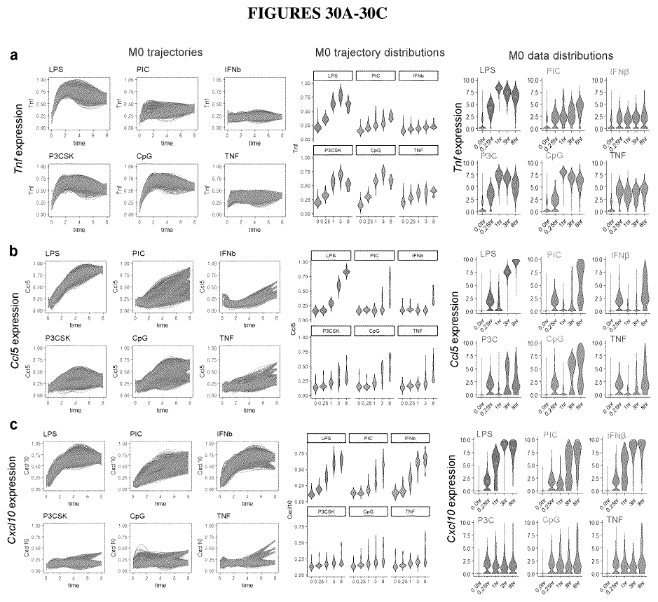

Applying scREAL-TIME to data from stimulated M0 macrophages, continuous single cell trajectories were recovered from 0 to 8 hours after macrophage stimulation, matching the range of the measured data. Taking Tnf for example, the method imputed the linkage of disconnected data points, providing the full time-course trajectory that recapitulated the magnitude and heterogeneity of the scRNAseq values at measured timepoints (FIG. 28C). The accuracy of the reconstructions of all individual genes was evaluated by comparing the mean and variance of the reconstructions to the measured mean and variance in the data, at each of the measured timepoints (FIGS. 29A-29B). Notably, some genes were more accurately reconstructed than others. While the trajectories shown for example gene Tnf appeared to reasonably match the data, this quantitation of fit revealed it to be one of the more poorly fit genes, with a relatively higher normalized RMSD for mean and variance (FIGS. 29C-29D). Plotting cell distributions from trajectory ensembles showed that the relatively poorer reconstruction accuracy for Tnf (nRMSD mean=0.31; nRMSD var=1.2) was due to higher variances at beginning and ends of the time-course and fast gene induction (FIG. 30A), though Tnf reconstructions did retain stimulus-specific information across timepoints. In contrast, for other cytokine genes Ccl5 (nRMSD mean=0.28, nRMSD var=0.43) and Cxcl10 (nRMSD mean=0.17, nRMSD var=0.64), mean induction dynamics and variance of distributions were closer fits to the data (FIGS. 30B-30C).

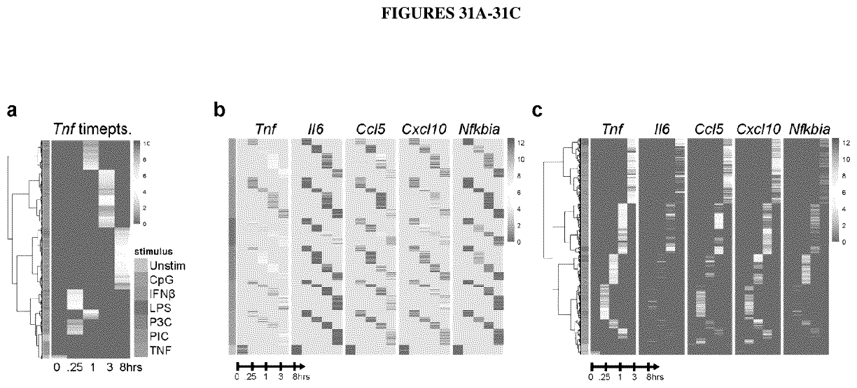

The trajectories imputed via scREAL-TIME allowed cells to be clustered using information from the entire time-course of gene expression, rather than solely at a particular timepoint. The dynamics of gene expression of a single cytokine, Tnf, was able to distinguish three stimulus groups, IFNβ, bacterial PAMPs, and TNF/PIC (FIG. 28D). In contrast, the maximum information in Tnf expression at a single timepoint 3 hours after stimulation was previously calculated to be 0.8 bits, not quite distinguishing even two states. Importantly, the correlated expression trajectories of all measured genes for each cell were recovered, enabling an assessment of stimulus-specific responses based on multigene time-courses. Hierarchical clustering was performed using the dynamic information from proinflammatory cytokines Tnf and Il6, lymphocyte chemokines Ccl5 and Cxcl10, and the NFκB negative feedback regulator Nfkbia. This multivariate combination of gene dynamics improved the distinguishability of stimuli, seemingly through complementary dynamical features of expression: cells stimulated with TNF vs. PIC, previously undistinguishable by Tnf dynamics alone, were now distinguishable by the late induction of Cxcl10 and Ccl5 activity in PIC-stimulated cells. Notably, measuring a single early timepoint such as 1 hour post-stimulation would not have captured this temporal distinction in gene expression (FIG. 28E). Hierarchical clustering on the time-point data of the same five genes did not well distinguish any stimuli (FIGS. 31A-31C).

### Dynamical Features of Single-Cell Gene Expression Trajectories Hold Stimulus Information

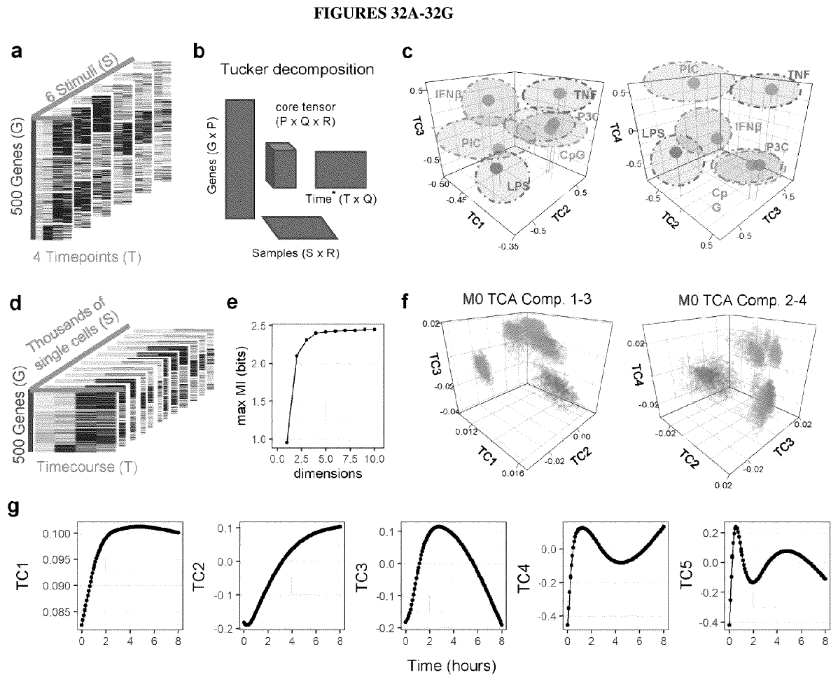

With the temporal expression trajectories of multiple genes within single cells available, it was asked whether and which dynamical features of these trajectories were important in enabling the distinction of stimuli. Each cell expresses hundreds of immune response genes simultaneously, which may provide information in combination, through the coordinated timing and amplitude of their expression. To first visualize this multidimensional array of data, tensor components analysis was performed, an unsupervised dimensionality reduction approach analogous to PCA for two-dimensions. Tensor decomposition of population-level time-series RNAseq data (genes×stimuli×time) (FIGS. 32A-32B) indicated that population average responses are distinct from each other (FIG. 31C). However, population averages fail to reveal the heterogeneous “clouds” of cells that may display wide distributions and heavy overlap among stimuli (FIG. 32C). Tensor decomposition was thus performed on all single cell imputed trajectories (genes×single cells×time) (FIGS. 32D-32F) and it was found that the top four components of this unsupervised dimensionality reduction approach held ˜2.4 bits of stimulus information (distinction of 5 stimuli), close to the theoretical maximum of 2.58 bits (distinction of all 6 stimuli) (FIG. 32E). Examining the time factors of the TCA, it was found that this stimulus-distinction relied on genes with clearly distinct dynamical patterns of gene expression (FIG. 32G).

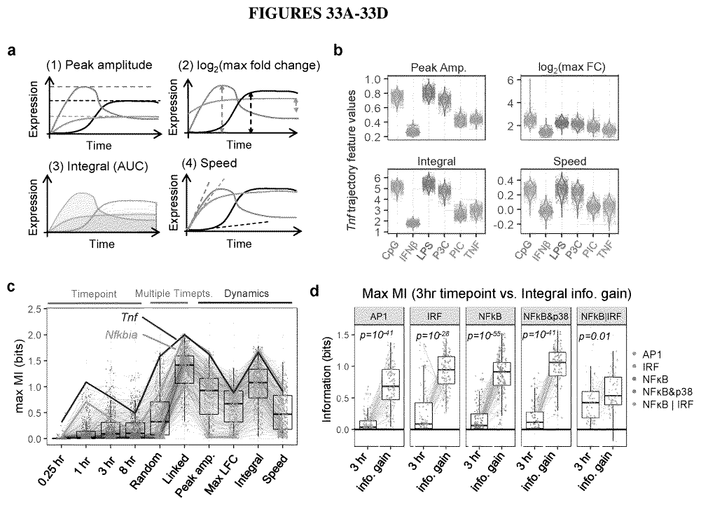

To quantify the dynamical patterns of expression trajectories, the presence of four dynamical features important in gene expression was considered: peak amplitude, max log fold change (max LFC), integral, and activation speed (FIG. 33A). For Tnf, the dynamical features of peak amplitude and integral were more stimulus-specific than fold change or activation speed, with less overlapping single cell distributions (FIG. 33B). To quantify the stimulus information contained in each dynamical feature across all genes, the maximum mutual information (MI) between the six-stimulus input and the output expression or feature distributions of each gene was calculated. Higher maximum MI indicates that the gene's dynamical feature is more stimulus-specific. The max MI at a single measured timepoint was consistently less than using multiple measured timepoints. Importantly, it was noted that using 4 randomly linked timepoints improved the max MI marginally compared to single timepoints, but 4 timepoints linked via scREAL-TIME allowed for significantly greater stimulus-specificity for all genes (FIG. 33C).

To examine how much the features of expression trajectories enable macrophage stimulus-specificity, the information content of each dynamical feature was assessed next. Information content in any particular feature was different for each genes, but it was found that “integral” and “peak amplitude” were on average the most stimulus-specific, while “max LFC” and “speed” were less informative (FIG. 33C). Certain genes more greatly relied on dynamical features to retain stimulus-specific information. For example, the NFκB target gene Nfkbia was less stimulus-specific when considering individual timepoints than when considering timepoints linked via scREAL-TIME or resulting dynamical features (FIG. 33C). This confirms the notion that dynamic expression is particularly important for Nfkbia, which encodes a NFκB feedback protein, to generate appropriate responses to immune threats.

It was noticed that for subsets of genes, the dynamic features provided much more stimulus information than a single timepoint, while other genes were associated with smaller gains in information. To investigate this further, the immune response genes were categorized based on which signaling pathways activate them. Interestingly, genes that were regulated by single transcription factors such as AP1, IRF, or NFκB showed an information gain of ˜1 bit, but genes regulated by an NFκBIIRF “sequential OR” gate showed gains of only ˜0.5 bits (FIG. 33D). This pattern suggested that the combinatorial activity of two transcription factors at the gene promoter may allow for stimulus distinction through combinatorial control with less reliance on the temporal dynamics of gene expression, while genes induced by a single transcription factor are more likely show stimulus-specificity through dynamic control (FIG. 33D).

### Single Cell Gene-Gene Correlations of Dynamical Features

In transcriptome measurements of a cell population, genes regulated by the same signaling pathway are induced with highly correlated patterns that can be clustered into regulatory modules. In transcriptome measurements of single cells, target genes of different signaling pathways may also be correlated due to a similar chromatin or signaling environment within each cell. To determine whether the extent of gene-gene correlations across single cells was dynamics-dependent, correlations of gene-pairs that are targets of the same or different transcription factors were compared, for either single-timepoint scRNAseq data or single-cell expression dynamical features.

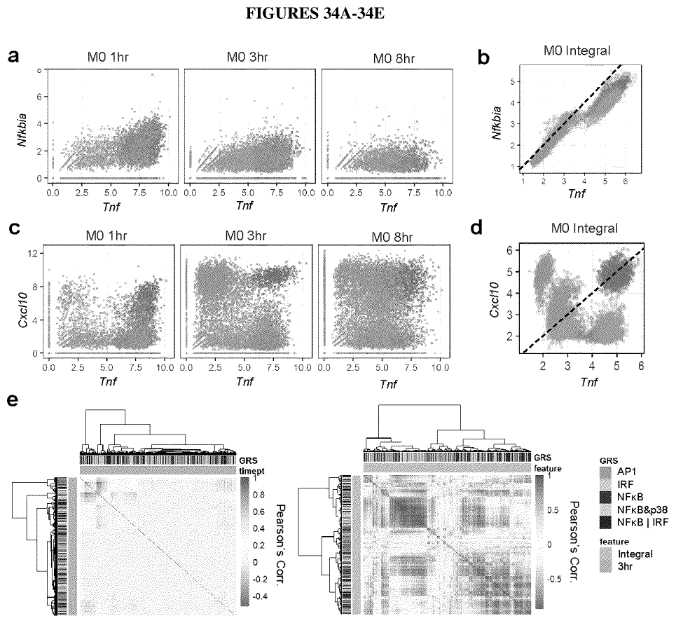

Two prominent NFκB regulated genes, Tnf and Nfkbia, showed weak to no correlation at any single response timepoint of 1 hr, 3 hrs, or 8 hrs (FIG. 34A). The lack of correlation at a single timepoint could be attributed to differences in the timing of activation: Tnf expression was more sustained throughout the time course while Nfkbia more frontloaded, and thus total expression was not well captured by any single timepoint. However, considering the dynamical feature “Integral” revealed high gene-gene correlations both across stimuli and within each stimulus condition (FIG. 34B). Examining the single-cell “Integral” values of these two genes also showed a shift for cells stimulated with CpG, P3C, or LPS that represented an increase in Tnf expression relative to Nfkbia. CpG, P3C, and LPS are immune ligands signal through the Myd88 to activate MAPKp38, resulting in an increase in NF03&p38-regulated Tnf expression relative to NFκB-only regulated Nfkbia. Only single-cell dynamical features could clearly reveal this subtle shift in the Tnf-Nfkbia correlation that reflected the known regulation of these two genes.

Two cytokine genes regulated by different signaling pathways were then examined, NFκB-regulated Tnf and NFκBlIRF-regulated Cxcl10 (FIG. 34C). Unlike Tnf and Nfkbia, the integrals of Tnf and Cxcl10 were not well correlated across stimuli, as Cxcl10 was strongly activated by IFNβ- or PIC-induced IRF signaling (FIG. 34D). However, within each stimulus, the Integrals of Tnf and Cxcl10 were still moderately correlated to an extent greater than evident from timepoint data (FIG. 34D), indicating that cells with high production of Tnf were also likely to produce higher levels of Cxcl10, through either shared activation of signaling pathways, similar gene regulatory mechanisms, or responsive chromatin environments. More globally, the correlation heatmap of all genes at a single timepoint of 3 hrs did not separate genes regulated by different regulatory strategies, while clustering on “Integral” across all genes recovered the information necessary to distinguish NFκB vs. IRF-regulated gene clusters (FIG. 34E). Analyzing the full dynamic trajectory of single-cell gene induction was thus critical for capturing the correlative relationship between gene-pairs, as well as the effect of individual cell states in providing similar chromatin or signaling environments for co-regulated transcription.

### Coordination of Dynamical Information Enables Stimulus-Specificity Across Macrophage Polarization States

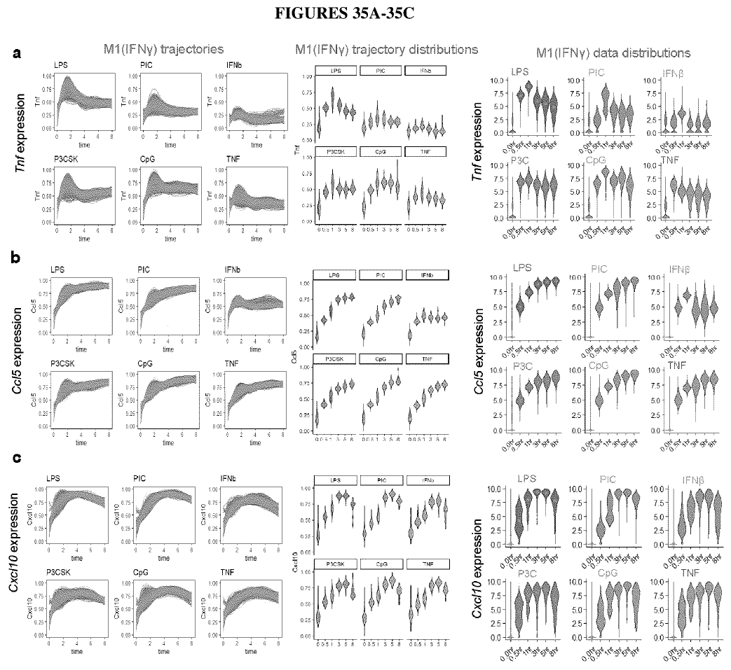

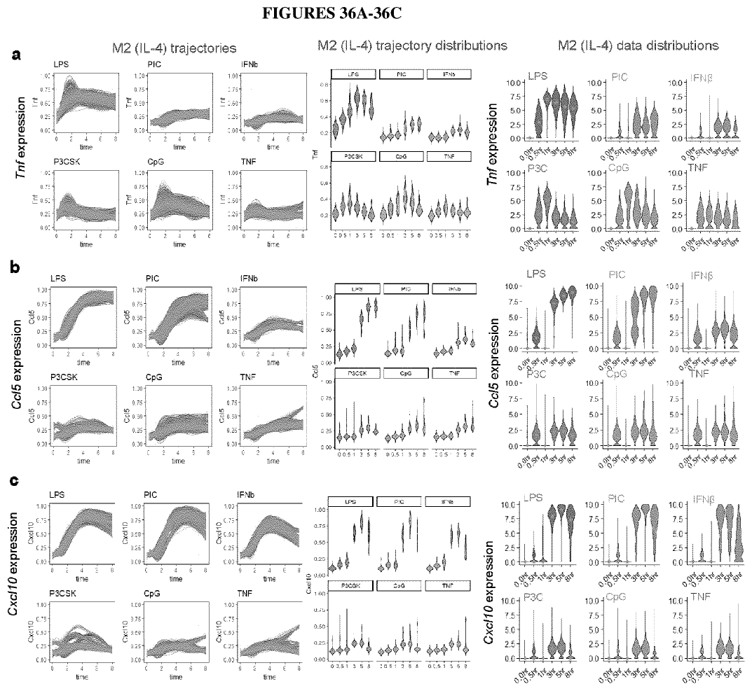

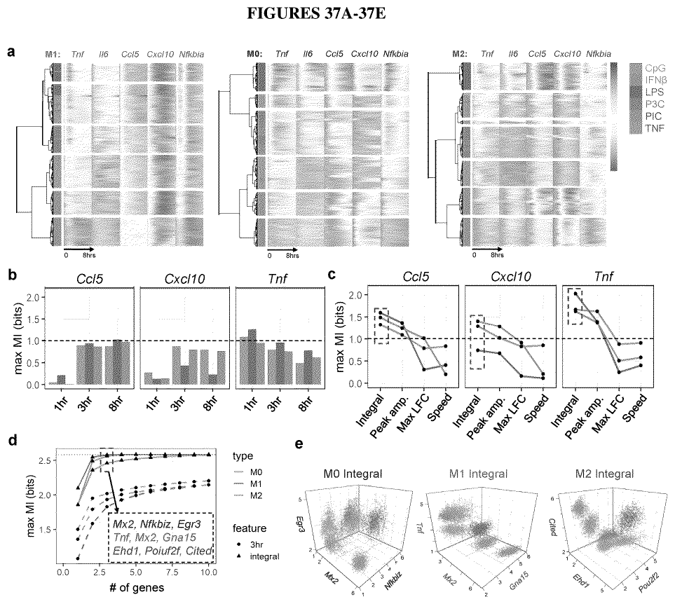

How do dynamical features of gene expression change when macrophages are polarized by microenvironmental cytokines? scREAL-TIME was next applied to time-series scRNAseq data from stimulated M1 (IFNγ) and M2 (IL4) macrophages and it was confirmed that distributions of imputed trajectories in polarized macrophages were also appropriate fits to measured data distributions at each timepoint (FIGS. 35-36). Initial inspection revealed distinct changes to the expression trajectories of several regulatory categories of immune response genes (FIG. 37A). For example, in response to bacterial PAMPS LPS, CpG, and Pam3CSK4, the NFκB target genes Tnf and Nfkbia both exhibited more transient activity in M1 (IFNγ) macrophages that was diminished by 3 hours, in contrast to more sustained activity in M0 macrophages. NFκBlIRF target genes Ccl5 and Cxcl10 were more rapidly induced in M1 (IFNγ) macrophages, while exhibiting much slower speed of induction in M2 (IL4) macrophages (FIG. 37A). Both M1 (IFNγ) and M2 (IL4) macrophage responses remained stimulus-specific for this subset of genes, but the specificity could be attributed to different aspects of gene dynamics.

To quantify polarization-dependent alterations to the stimulus-specificity of gene expression trajectories, the stimulus information contained within the expression of three critical immune response cytokines was examined Tnf, Ccl5, and Cxcl10. At any single timepoint, stimulus-information did not exceed 1.2 bits for any polarization state (FIG. 37B). The effect of polarizing cytokines was also gene-specific, with IFNγ polarization decreasing the stimulus-information in Cxcl10 expression across all timepoints but increasing the information in Tnf expression (FIG. 37B). In contrast, the information in the “integral” dynamical feature exceeded 1.2 bits for all three genes. Interestingly, for Cxcl10, IFNγ polarization caused loss of dynamical information across all dynamical features, but for Tnf and Ccl5, IFNγ effects were feature-specific (FIG. 37C): for these two genes, the stimulus-information in “integral” was enhanced by an IFNγ microenvironment, but information in “max LFC” and “speed” were diminished. As “max LFC” and “speed” are features determined early in the time-course, while “integral” requires the full timecourse, these results suggest that IFNγ polarization causes more promiscuous production of Tnf and Ccl5 early on, but stimulus-specific expression at later phases.

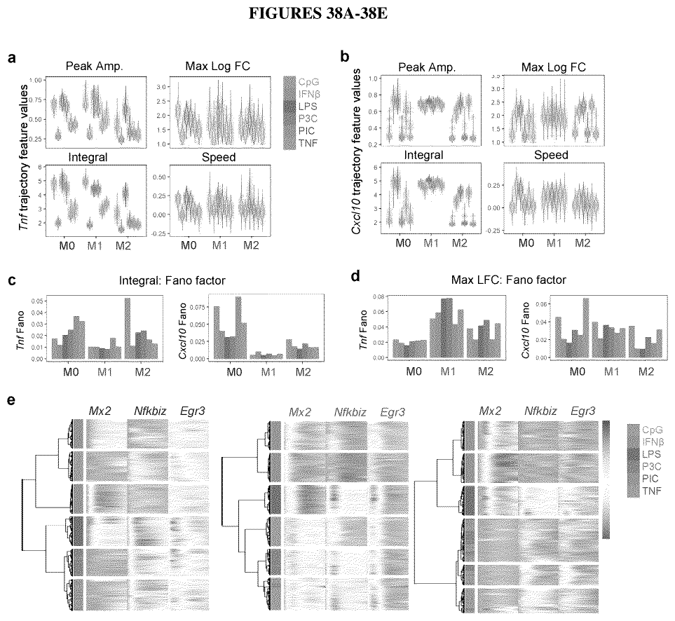

To uncover whether gene-specific changes to stimulus-specificity were due to changes to the mean or variance of dynamical features, the single-cell distributions of dynamical features for Tnf vs. Cxcl10 were plotted. The Integral of Tnf expression had similar averages in M1 (IFNγ) and M0 macrophages, but single-cell variability was decreased (FIG. 38A), thereby increasing the stimulus-specificity of Tnf trajectory Integrals. On the other hand, though IFNγ also reduced single cell heterogeneity in the Integral of Cxcl10 trajectories, average values that were much more similar across stimuli in M1 (IFNγ) macrophages, thereby decreasing stimulus-specificity (FIG. 38B). Thus, polarization-induced changes to the stimulus-specificity of expression dynamical features for each gene was highly dependent on not only changes in averages but also the degree of cell-to-cell heterogeneity (FIGS. 38C-38D).

To investigate the coordination of multiple genes in specifying stimulus-identity, the most informative gene combination when considering dynamical features rather than single timepoints was next identified. Indeed, for the dynamical feature “Integral” in M0 macrophages, max MI approached ˜2.5 bits with just three genes (Mx2, Nfkbiz, Egr3), near the theoretical maximum of 2.58 bits (distinguishing 6 stimuli). A max MI near the theoretical ceiling could also be achieved with three genes for M1 (IFNγ) (Mx2, Tnf, Gna15) and M2 (IL4) macrophages (Cited, Ehd1, Pou2f2), in contrast to gene combinations a single timepoint of 3 hours which reached at most 2 bits of information (FIG. 37D). Plotting the Integral of the selected genes corroborated the conclusion that the dynamic features separated the six stimuli distinctly, though some stimulus information was still lost due to overlap in CpG and P3C distributions (FIG. 37E). Thus, while stimulus-specificity was diminished when examining the trajectory of a single gene like Cxcl10, the stimulus-response specificity of polarized macrophages was not diminished when considering multiple gene trajectories in combination.

Which dynamical characteristics of these gene combinations provide for the stimulus-specificity? Hierarchical clustering of cell trajectories indicated that the genes selected for M0 macrophages contained complementary dynamical features in their stimulus-response trajectories. For example, the interferon-regulated antiviral gene Mx2 was activated in response to both LPS and IFNβ, but was induced with slower speed in response to LPS (FIG. 38E). Across polarization states, M1 (IFNγ) macrophages activated Mx2 more rapidly compared to M0 and M2 (IL4) macrophages more slowly, but across all polarization states, the relative delay in Mx2 induction in LPS vs. IFNβ response was maintained. This suggested that although different polarization states have similar abilities to distinguish stimuli, which genes and which dynamical features mediate this specificity is polarization-specific—thus charting the changes to dynamical feature distributions is critical for understanding the alternate biological functions of different polarization states.

### The Response Specificity Profile of Dynamical Features Enables Comparisons of Stimulus-Pair Distinctions Across Polarization States

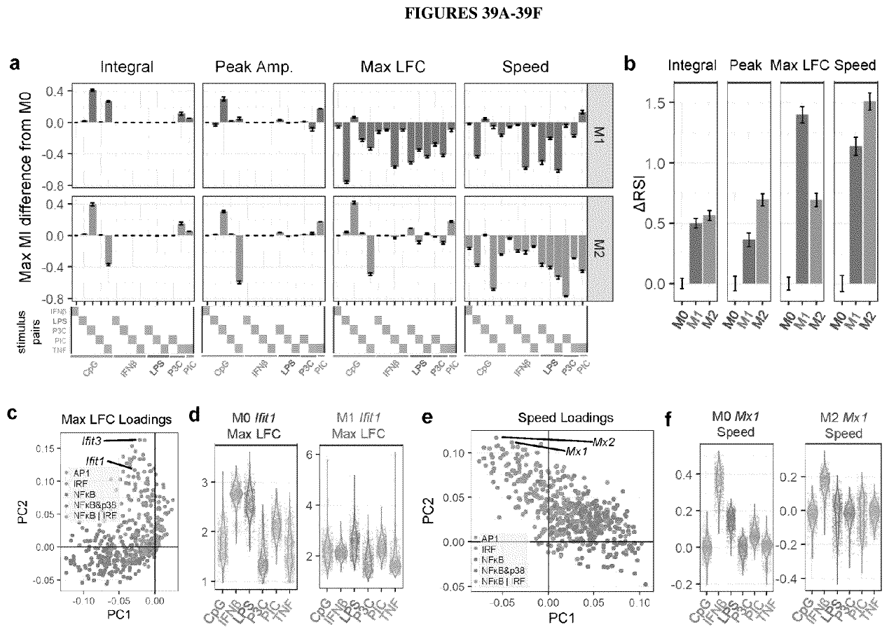

To further identify which genes most strongly differed in dynamical features across polarization states, and which stimulus-pair distinctions were affected by these changes, the Stimulus Response Specificity Profile for each dynamical feature was calculated. The Response Specificity Profile is an unsupervised characterization of the distance between pairs of stimulus-response distributions. By applying the Response Specificity Profile to dynamical features, differences in stimulus-response specificity (SRS) across macrophage states can be attributed to dynamical features that particularly affect the distinction between certain pairs of stimuli. It was found that M1 (IFNγ) macrophage responses lost SRS most prominently in the Max LFC feature across most stimulus pairs, for example especially between CpG and LPS. In contrast, M2 (IL4) macrophage responses lost SRS most prominently in the Speed dynamical feature, such as between P3C and IFNβ (FIG. 39A) Summing the deviation in M1 (IFNγ) and M2 (IL4) macrophages from M0 macrophages across all stimulus pairs resulted in a summary metric, the ΔResponse Specificity Index (ΔRSI), for each dynamical feature (FIG. 39B). The ΔRSI revealed that while stimulus-specific information contained in the total expression (integral) and peak expression were affected by polarization, fold change and timing showed greater changes. M1 (IFNγ) macrophage SRS differed from M0 most strongly in the Max LFC feature of gene expression dynamics, and both M1 (IFNγ) and M2 (IL4) macrophages differed from M0 in the SRS of activation Speed (FIG. 39B). Inspection of expression trajectories revealed (FIG. 37A, FIG. 38E) that M1 (IFNγ) macrophages lost specificity in the Speed feature due to more uniform increases in gene activation speed across stimuli, while M2 (IL4) macrophages lost specificity through uniform decreases in speed.

Using the gene loadings underlying the Response Specificity Profile and ΔRSI, the genes contributing most to loss of SRS in these two particular features of Max LFC and Speed were next identified. For the Max LFC dynamical feature, a group of IRF and NFκBlIRF genes were weighted most strongly, among them Ifit3 and Yid (FIG. 39C). Plotting Ifit1 Max LFC for all single cells revealed that Ifit1 was highly stimulus-specific in M0 macrophages, but this specificity was lost in M1 (IFNγ) macrophages, most prominently between two bacterial stimuli like CpG and LPS (FIG. 39D), as predicted in the Response Specificity Profile. Similarly, gene loadings for the dynamical feature Speed identified a group of genes including Mx1 and Mx2 (FIG. 39E), as contributing the most to the loss of Speed-dependent SRS in M2 (IL4) macrophages compared to M0 macrophages (FIG. 39F). Again, the violin plots for Mx1 Speed corroborated that stimulus-pairs such as P3CSK-PIC (bacterial vs. viral) were most affected, matching the result given by the Response Specificity Profile (FIG. 39F). These metrics thus systematically identified gene expression dynamical features whose stimulus-specific information was most altered by each polarization condition, the stimulus-pairs that became less distinguishable as a result, and the genes most heavily involved.

### Identification of Cell Functional State Based on Gene Expression Response Dynamics

The context-dependent cell state of macrophages is typically assessed by protein markers or single cell transcriptomics in their quasi-steady state. Yet, macrophage functions are deployed stimulus-specifically, and how macrophages respond to stimuli is a function of the microenvironmental context. Therefore, in addition to containing accurate stimulus information, gene expression dynamics may also allow more accurate identification of a cell's functional state. Assessing stimulation-response dynamics integrates steady-state epigenetic information and probes the functional outcome of microenvironmental perturbations for each cell.

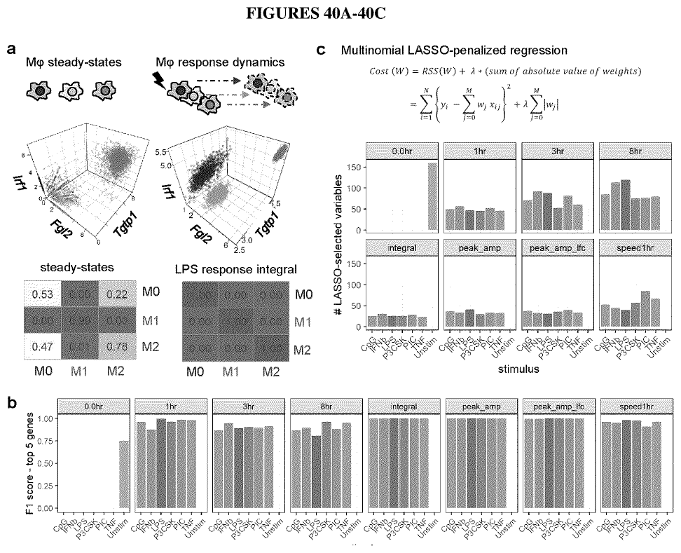

To examine the difference between steady-state measurements and response-dynamical features in distinguishing polarization states, the top three genes that individually best distinguished unstimulated M0, M1, and M2 macrophages (Irf1, Fgl2, Tgtp1) were chosen and have their expression values plotted. It was found that steady-state expression values poorly distinguished M0 and M2 macrophages, while the LPS response integral of the same three genes perfectly distinguished the three polarization states (FIG. 40A), suggesting that information contained in expression dynamics could much more accurately specify a single cell's state.

The top five genes identified by max MI were used to build statistical learning classifiers that predicted cell polarization states, based on 1) steady-state (0 hours) expression values, 2) single timepoint response expression values, or 3) response dynamical feature values (FIG. 40B). It was found that prediction accuracy (F1 score) was higher for dynamical features than for steady-state values, across any stimulus used. In addition, F1 scores for cell state prediction were lower when considering single timepoint measurements vs. full time course, but still higher than steady state values (FIG. 40B), supporting the hypothesis that perturbation responses, and in particular the full gene expression dynamics, more reliably reveal cell state.

The top 5 genes used to predict macrophage polarization state using the Integral feature are:

| LPS | polyI:C | IFNβ | P3CSK4 | CpG | TNF |
|---|---|---|---|---|---|
| Cxcl11 | Socs1 | Socs1 | Slamf8 | Slamf8 | Olr1 |
| Abtb2 | Myo1b | Plac8 | Socs1 | Arrdc4 | Htr2a |
| Tmem200b | Rasgrp1 | Kdr | Nos2 | Glipr2 | Myo1b |
| Arrdc4 | Fgl2 | Il12rb1 | Dusp4 | Olr1 | Rasgrp1 |
| Hopx | Gm4951 | Tnfsf8 | Myo1b | Upp1 | Slamf8 |

To examine the number of genes needed in each scenario that minimizes the prediction error, a multinomial LASSO (Least Absolute Shrinkage and Selection Operator)-penalized regression model was built on all measured genes. Importantly, LASSO regularization shrinks the coefficients of some variable to zero, thus allowing elimination of many predictors from the model to avoid overfitting the data. It was found that performing LASSO-regularized regression on steady-state expression values resulted in a model containing ˜150 genes, while models built on single time-point response values contained ˜50-120 genes, depending on the timepoint and the stimulus used (FIG. 40C). On the other hand, models built using dynamical features required only −20-40 genes to minimize the residual sum of squares of the predicted values (FIG. 40C), again pointing to the increased information content of gene response dynamics in specifying single-cell functional states.

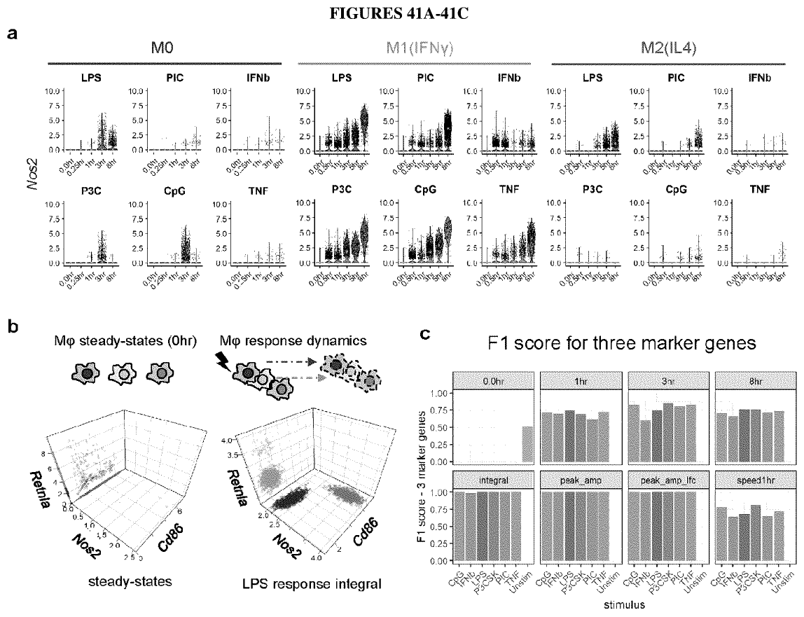

It was assessed whether dynamic features provided more information about polarization state even for canonical marker genes of M1 (IFNγ) or M2 (IL4) polarization. For example, the well-known M1 macrophage marker Nos2 had on average higher expression at early timepoints in M1 macrophages, but single cell expression distributions of Nos2 overlapped across polarization states such that not all single cells could be identified as M1 based on this marker alone (FIG. 41A). However, plotting the expression values of Nos2 (M1 marker), Cd86 (M1 marker), Retnla (M2 marker) at the unstimulated steady-state, versus their LPS response integral, it was found that the response integral much more distinctly distinguished all three macrophage polarization states (FIG. 41B). A classifier for polarization state using these three genes again revealed that a single stimulus-response timepoint identified cell state better than in Unstimulated cells, and using the dynamical features, especially Integral, peak amplitude, or max fold change, further drastically improved polarization state identification (FIG. 41C).

### DISCUSSION

The present example showed that temporal trajectories of gene expression within single cells contain rich information about external stimuli and the functional state of the cell. Single-cell expression trajectories of just a handful of genes provided enough information for near perfect stimulus-response specificity, or for high accuracy identification of cell functional states. Stimulus-response gene expression is dynamic and heterogeneous, but current single-cell RNA measurement technology has not been able to capture both these characteristics simultaneously, as scRNAseq is destructive to cells. The newly developed single-cell trajectory imputation method scREAL-TIME revealed the heterogeneity of dynamic responses, thus quantifying macrophage stimulus-response specificity more realistically than could be done from previous time-point scRNAseq. When considering dynamical information, stimulus-response specificity was much higher than previously estimated, and expression trajectories were found to be both dramatically and subtly altered by the microenvironmental context of polarizing cytokines. Indeed, it was found that the stimulus-response trajectories of macrophages responding from different cytokine contexts were so distinctive that response dynamics were much more informative of the functional cell state than steady-state mRNA abundance measurements.

In developing scREAL-TIME, it was aimed to reduce the effect of technical noise (e.g., drop-outs in scRNAseq) and provide the least complex ensemble of trajectories that accounted for the heterogeneous datasets of all measured time points. The method first relied on PCA to compress the information from 500 genes per cell into a cell score, with gene-gene correlations maintained in the PCA loadings. To reduce the impact of technical noise and to decrease computational time, cells at each timepoint were binned into cell archetypes. While specifying too few archetypes may miss outlier behavior of rare cells, it was determined that 20 clusters per timepoint appropriately approximated the distributions of the data, as 20⁵ (5 timepoints) equated 3.2 million possible paths, far exceeding the number of cells measured. Thus, the idea of cell archetypes reduced the impact of technical noise at each timepoint, while still maintaining sufficient coverage of possible across-timepoint biological variability. Using weighted random walks to link cell archetypes over timepoints resulted in trajectories that were both data-driven and biophysically plausible, as cells transcriptomically more similar across timepoints were linked with greater probability and no trajectories were imputed with rapidly oscillatory characteristics that contradicted knowledge of known mRNA half-lives. This probabilistic approach to linking data across timepoints also ensured that all permutations of paths were possible, but some were far more unlikely given the data. Identifying the trajectory paths for the entire cell in the latent space and then recovering individual gene trajectories by multiplying the PCA loadings matrix also allowed cellular gene-gene correlations to be preserved, which would not be the case if trajectory paths were identified for each gene independently.

scREAL-TIME was developed to address a gap left by prior computational and experimental methods that have aimed to analyze single-cell response dynamics Single-cell real-time trajectories may be imputed by fitting mechanistic models to measured time-series single-cell data distributions. This approach has been successfully applied to data of signaling proteins using models of signaling networks, but models of gene expression remain coarse and insufficiently predictive. Further, the gene expression data consists of hundreds of data points (500 genes) per cell, i.e., 500 single-cell distributions per time-point, posing additional challenges in fitting mechanistic models. Experimentally, the primary methodology for measuring single-cell gene expression trajectories is the MS2 reporter which allows live cell imaging of fluorescently tagged mRNA transcripts. However, this technology only images one or two genes at a time, precluding analysis of correlations or biological importance of genes expressed in combination. In addition, it requires genetic engineering of the genes of interest, which requires immortalized cells (as opposed to primary macrophages) and may affect expression dynamics. While the MS2 reporter provides higher time-resolution (5 minute intervals) than scREAL-TIME (15 minute intervals at the smallest, with even smaller being likely cost-prohibitive), it was aimed to make more frequent measurements at early phases of the immune response when immune response transcriptomes are known to be changing rapidly. In applying scREAL-TIME to other datasets, interpolating over larger measurement intervals of multiple hours should assume that single-cell transcriptomes are stable on the order of minutes during that time frame.

Applying scREAL-TIME to macrophages stimulated with diverse immune stimuli revealed new information, as the expression dynamics of immune-response genes could be analyzed at the single cell level. Each gene differed in their dynamic patterns of induction as well as their level of single-cell heterogeneity, which could be quantified by decomposing the trajectories into dynamical features such as integral, fold change, peak amplitude, and speed, an approach that has proved informative in cell signaling studies. The stimulus-response specificity of single genes or combinations of genes was determined. For most genes, the trajectory Integral was found to correlate best with the stimulus. Unlike features like speed or max fold change, Integral combines information from the entire time-course and may thus be most robust to both technical noise and biological heterogeneity. As cells must adapt to changes in their environment, stimulus-response specificity is an important biological characteristic, particularly in immune sentinel cells such as macrophages. Several studies have investigated the stimulus-information transmitted in biochemical signaling networks through live cell imaging of kinase activities or transcription factor nuclear translocation, including NFκB in macrophages. The trajectory vector of NFκB signaling was shown to distinguish different doses of ligands much more accurately than single timepoints of NFκB activity, and dynamical features NFκB trajectories were identified to best distinguish different ligands, analogous to what was found with gene expression. However, estimations of the stimulus-information within the dynamics of a single transcription factor like NFκB was consistently lower than that calculated for the dynamics of the most informative gene, as many of the gene trajectories examined are the outcome of multiple non-redundant signaling pathways that operate on genes with different timescales, such as Tnf (NFκB&p38) or Cxcl10 (NFκB|IRF). In addition, the expression dynamics obtained via scREAL-TIME consist of hundreds of trajectories per cell, enabling identification of complementary stimulus-specific gene dynamics, while measuring the trajectories of more than a few signaling proteins simultaneously in a single cell has not been feasible.

The hallmarks of healthy macrophage function have been described as stimulus-response specificity, context-dependence, and memory of prior exposures. These properties either define or reveal the functional state of macrophages: defined by context and history, and revealed by measuring stimulus-responses. In macrophages, stimulus-response gene expression dynamics are determined by 1) the activities of upstream signaling effectors (e.g., transcription factors NFκB, IRF, AP-1), whose abundances and nuclear translocation dynamics are strongly affected by contextual cytokines, and 2) the mechanisms and molecules available to interpret information within transcription factor activation, including chromatin opening mechanisms, nucleosome dynamics, and histone modifications, which can also be significantly altered by contextual cytokines that either prime or repress gene regulatory elements. For example, an IFNγ polarization context not only increases the nuclear availability of NFκB but also alters the epigenomic landscape of open chromatin and histone modifications. Thus, the cell's response to stimuli not only encodes information about the stimulus, but also about the cell's functional state.

Why might the functional state of a cell be predicted more accurately when accounting for dynamical features of expression rather than steady-state gene expression? Molecularly, response dynamics are more informative than steady-state omics profiles (chromatin, RNA, protein, etc.) because the functional state of a cell is not only defined by abundances of molecules but also by the rate constants that determine synthesis, degradation, association, dissociation and catalysis. For example, two cells may have the exact same amount of a ligand receptor at a particular time, but if one cell has higher synthesis or degradation of the receptor than the other, the two would respond differently to the same ligand concentration. Measuring responses integrates both the kinetic and abundance pieces of a cell's molecular profile at the time of stimulation, including availability of receptors, activities of kinases, and dynamics and localization of transcription factors. Capturing responses at the level of gene expression dynamics also captures the cell's chromatin state. Thus, the method of obtaining single cell inducible gene expression trajectories categorizes cell states in a manner unobtainable from any current standard steady-state cell profiling methods and allows a more precise phenotyping of the functional state than single timepoint response data.

Obtaining kinetic information for phenotyping the functional states of other cell types poses several challenges. Producing temporal trajectories is resource intensive as it requires multiple timepoints following a perturbation. In addition, macrophages respond to hundreds of possible stimuli, and stimuli that distinctly activate different combinations of signaling pathways were chosen herein, but the choice of stimulus may be more limited for other cell types. In theory, stimuli that perturb specific regulatory networks, rather than broad-acting drugs that do not act gene- or pathway-specifically, may provide more refined definitions of functional states through greater diversity in the dynamical features of each gene. Ideally then, stimuli should target physiological or pathological pathways crucial to the function of the cell being tested. For example, cancer cells can be treated with small molecule drugs inhibiting cell cycle or cytokines mimicking immunotherapy, Androgen Receptor+ prostate cancer with androgens, or adipocytes with fatty acids/sterols that activate lipid receptors/LXRs (liver X receptors). Other surrogate measurements for obtaining single cell kinetic information have been proposed, such as spliced vs. unspliced RNA, mRNA vs. protein, or mRNA vs. ATACseq data. However, the technical noise of many of these measurements is substantial and may thus deteriorate the additional information gained through these modalities.

The present study was focused on the dynamics of single-cell inducible gene expression in macrophages, key to the initiation of immune responses. Response trajectories were central to providing the high stimulus-response specificity that is essential to macrophage function, and supplied the kinetic information necessary for defining cell functional states. Future applications of defining cell states by response trajectories could allow more robust clustering and subtyping for macrophages derived from undefined cytokine environments of inflammatory diseases, or enable the identification of rare subclusters of single cells in other diseases where the function of outlier cells is pivotal, such as the responses of individual cancer cells to drugs or cell damage signals.

### Example 16

### Using scREAL-TIME to Guide Therapy

A data bank comprising scREAL-TIME data collected from various patient populations is created, including healthy patients, patients over the time course of their diseases, and patients who have undergone various types of therapy for each disease. It is envisioned that blood draws for routine or patient monitoring will undergo scREAL-TIME testing and an anonymous data bank with such data and patient histories will be amassed. Such scREAL-TIME bank provides the basis for extracting diagnostic and prognostic information and guiding therapeutic intervention, both for therapies that may be effective and those likely to be ineffective.

In one particular use, the data bank is populated from data on transplant recipients, such as kidney, liver or lung transplant recipients, their organ rejection status over time and following treatment with antirejection drugs. A query of the data bank with the new patient's scREAL-TIME assessment provides a recommended therapeutic regimen.

In other examples, uses include monitoring tumor progression, response to anti-inflammatory therapy in autoimmune disease, and progression of autoimmune diseases. Non-limiting examples of autoimmune diseases include but are not limited to rheumatoid arthritis, juvenile dermatomyositis, psoriasis, psoriatic arthritis, sarcoidosis, lupus, Crohn's disease, eczema, vasculitis, ulcerative colitis and multiple sclerosis.

In another example, the effectiveness of tumor immunotherapy can be monitored.

In another example, the health status of a patient with a persistent infection such as methicillin-resistant Staphylococcus aureus (MRSA) can be identified, and adjustment of the therapeutic course made based on scREAL-TIME assessment and comparison with similar patients in the data bank.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for treating a subject having a condition or disease, comprising the steps of:
a. determining the responsiveness of the subject's innate immune system by a method comprising:
i. obtaining a blood sample from said subject;
ii. exposing monocytes or monocyte-derived cells derived from said blood samples to one or a combination of stimuli selected from among cytokines, pathogen-associated molecular patterns, and damage-associated molecular patterns;
iii. determining responses using genome wide RNA profiling, single cell mRNA expression of one or more of stimuli-related genes, or protein expression, in response to the stimuli in the monocytes or monocyte-derived cells (e.g., macrophages, dendritic cells); and
iv. determining a response specificity score characterizing the responsiveness of the subject's innate immune system, said determination comprises use of information theoretic and machine learning methodologies;
b. using the subject's response specificity score, querying an information repository comprising (i) innate immune system responsiveness of a plurality of healthy subjects and patients having the condition or disease, and (ii) a correlation between said innate immune system responsiveness and a therapeutic outcome of said patients to one or more therapeutic regimens, and identifying a correlation value for said subject; and
c. treating the subject with a therapeutic regimen when said correlation value indicates a positive therapeutic benefit for the subject; or avoiding treating the subject with a therapeutic regimen when said correlation value indicates an absence of or a negative therapeutic benefit for the subject.

2. The method of claim 1, wherein the blood sample comprises peripheral blood mononuclear cells.

3. The method of claim 1 wherein the monocyte or monocyte-derived cells are macrophages or dendritic cells.

4. (canceled)

5. The method of claim 1 wherein the determining responses is at a single time point after exposing, or at a plurality of time points after exposing.

6.-12. (canceled)

13. The method of claim 1 wherein the determining the response specificity score is based on a plurality of time points after exposing.

14. (canceled)

15. The method of claim 5 wherein the determining responses for a plurality of timepoints comprises integral, fold change, peak amplitude, speed, or any combination thereof.

16.-20. (canceled)

21. The method of claim 1 wherein the stimuli comprise LPS, polyI:C, IFNβ, P3CSK4, CpG and TNF.

22. The method of claim 1 wherein the stimuli consist of LPS, poly I:C, IFNβ, P3CSK4, CpG and TNF.

23. The method of claim 1 where a no stimulus control is included.

24.-25. (canceled)

26. The method of claim 1 wherein the wherein the stimuli-related genes evaluated for each stimulus are:

| LPS | polyl:C | IFNβ | P3CSK4 | CpG | TNF |
|---|---|---|---|---|---|
| Cxcl11 | Socs1 | Socs1 | Slamf8 | Slamf8 | Olr1 |
| Abtb2 | Myo1b | Plac8 | Socs1 | Arrdc4 | Htr2a |
| Tmem200b | Rasgrp1 | Kdr | Nos2 | Glipr2 | Myo1b |
| Arrdc4 | Fgl2 | Il12rb1 | Dusp4 | Olr1 | Rasgrp1 |
| Hopx | Gm4951 | Tnfsf8 | Myo1b | Upp1 | Slamf8 |

27. The method of claim 1, wherein determining said response specificity score comprises a machine learning algorithm that uses t-distributed stochastic neighbor embedding (t-SNE).

28. The method of claim 1 wherein scREAL-TIME analysis provides the response specificity score.

29. The method of claim 28 wherein scREAL-TIME comprises the steps of dimensionality reduction, k-means clustering, weighted random walks, spline fitting and recovering gene trajectories.

30. The method of claim 1, wherein determining said response specificity score comprises a pre-established relationship between responses of said stimuli-related genes to said stimuli, a pattern of expression among signaling systems, a pattern of expression of gene regulatory systems, a temporal progression of any of the foregoing, or combinations thereof.

31. The method of claim 1 wherein the polarization state of macrophages in the sample are determined.

32.-36. (canceled)

37. The method of claim 1 wherein the macrophage polarity stimuli-related genes evaluated for each stimulus are:

| LPS | polyl:C | IFNβ | P3CSK4 | CpG | TNF |
|---|---|---|---|---|---|
| Cxcl11 | Socs1 | Socs1 | Slamf8 | Slamf8 | Olr1 |
| Abtb2 | Myo1b | Plac8 | Socs1 | Arrdc4 | Htr2a |
| Tmem200b | Rasgrp1 | Kdr | Nos2 | Glipr2 | Myo1b |
| Arrdc4 | Fgl2 | Il12rb1 | Dusp4 | Olr1 | Rasgrp1 |
| Hopx | Gm4951 | Tnfsf8 | Myo1b | Upp1 | Slamf8 |

38. The method of claim 1, wherein said information repository comprises innate immune system responsiveness of said patients at one or more time points of said condition or disease.

39. The method of claim 1, wherein said condition or disease is cancer, inflammatory disease, autoimmunity, high BMI, transplant rejection, tumor progression, tumor immunotherapy, sepsis, infection, or advanced age.

40. The method of claim 39 wherein the autoimmune diseases are rheumatoid arthritis, juvenile dermatomyositis, psoriasis, psoriatic arthritis, sarcoidosis, lupus, Crohn's disease, eczema, vasculitis, ulcerative colitis or multiple sclerosis.

41. A method of determining innate immune system responsiveness of a subject, comprising:
i. obtaining a blood sample from said subject;
ii. exposing monocytes or monocyte-derived cells (e.g., macrophages, dendritic cells) derived from said blood samples to one or a combination of stimuli selected from among cytokines, pathogen-associated molecular patterns, and damage-associated molecular patterns;
iii. determining responses using genome wide RNA profiling, single cell mRNA expression of one or more of stimuli-related genes, or protein expression, in response to the stimuli in the monocytes or monocyte-derived cells (e.g., macrophages, dendritic cells); and
iv. determining a response specificity score characterizing the innate immune system responsiveness of said subject, said determination comprises use of information theoretic and machine learning methodologies.

42.-77. (canceled)
